SEQUENCE LISTING (53 sequences)

SEQ ID NO: 1 - DNA, 81 bp, synthetic construct
  source 1..81: mol_type = other DNA
  misc_feature 1..81: note = reference sequence
ggcaccagccagctgagccaattcatggaccagaacaacccgctgtcggggttgacccacaagcgccgactgtcggcgctg

SEQ ID NO: 2 - DNA, 15 bp, synthetic construct
  source 1..15: mol_type = other DNA
  misc_feature 1..15: note = wild-type katG sequence
gcgatcaccagcggc

SEQ ID NO: 3 - DNA, 15 bp, synthetic construct
  source 1..15: mol_type = other DNA
  misc_feature 1..15: note = katG S315T variant sequence
gcgatcaccaccggc

SEQ ID NO: 4 - DNA, 15 bp, synthetic construct
  source 1..15: mol_type = other DNA
  misc_feature 1..15: note = katG S315N variant sequence
gcgatcaccaacggc

SEQ ID NO: 5 - DNA, 15 bp, synthetic construct
  source 1..15: mol_type = other DNA
  misc_feature 1..15: note = katG S315I variant sequence
gcgatcaccatcggc

SEQ ID NO: 6 - DNA, 15 bp, synthetic construct
  source 1..15: mol_type = other DNA
  misc_feature 1..15: note = katG S315R variant sequence
gcgatcaccagaggc

SEQ ID NO: 7 - DNA, 15 bp, synthetic construct
  source 1..15: mol_type = other DNA
  misc_feature 1..15: note = katG S315G variant sequence
gcgatcaccggcggc

SEQ ID NO: 8 - DNA, 15 bp, synthetic construct
  source 1..15: mol_type = other DNA
  misc_feature 1..15: note = katG S315L variant sequence
gcgatcaccctcggc

SEQ ID NO: 9 - DNA, 15 bp, synthetic construct
  source 1..15: mol_type = other DNA
  misc_feature 1..15: note = katG S315T+A312V variant sequence
gtgatcaccaccggc

SEQ ID NO: 10 - DNA, 15 bp, synthetic construct
  source 1..15: mol_type = other DNA
  misc_feature 1..15: note = katG S315T+G316D variant sequence
gcgatcaccaccgac

SEQ ID NO: 11 - DNA, 15 bp, synthetic construct
  source 1..15: mol_type = other DNA
  misc_feature 1..15: note = katG S315T+G316D+A312V variant sequence
gtgatcaccaccgac

SEQ ID NO: 12 - DNA, 200 bp, synthetic construct
  source 1..200: mol_type = other DNA
  misc_feature 1..200: note = Wild-type drug-susceptible target
atctggtcggccccgaacccgaggctgctccgctggagcagatgggcttgggctggaagagctcgtatggcaccggaaccggtaaggacgcgatcaccagcggcatcgaggtcgtatggacgaacaccccgacgaaatgggacaacagtttcctcgagatcctgtacggctacgagtgggagctgacgaagagccctgct

SEQ ID NO: 13 - DNA, 89 bp, synthetic construct
  source 1..89: mol_type = other DNA
  misc_feature 1..89: note = Mutant target
ctcgtatggcaccggaaccggtaaggacgcgatcaccaccggcatcgaggtcgtatggacgaacaccccgacgaaatgggacaacagtt

SEQ ID NO: 14 - DNA, 89 bp, synthetic construct
  source 1..89: mol_type = other DNA
  misc_feature 1..89: note = Mutant target
ctcgtatggcaccggaaccggtaaggacgcgatcaccaacggcatcgaggtcgtatggacgaacaccccgacgaaatgggacaacagtt

SEQ ID NO: 15 - DNA, 89 bp, synthetic construct
  source 1..89: mol_type = other DNA
  misc_feature 1..89: note = Mutant target
ctcgtatggcaccggaaccggtaaggacgcgatcaccatcggcatcgaggtcgtatggacgaacaccccgacgaaatgggacaacagtt

SEQ ID NO: 16 - DNA, 89 bp, synthetic construct
  source 1..89: mol_type = other DNA
  misc_feature 1..89: note = Mutant target
ctcgtatggcaccggaaccggtaaggacgcgatcaccagaggcatcgaggtcgtatggacgaacaccccgacgaaatgggacaacagtt

SEQ ID NO: 17 - DNA, 89 bp, synthetic construct
  source 1..89: mol_type = other DNA
  misc_feature 1..89: note = Mutant target
ctcgtatggcaccggaaccggtaaggacgcgatcaccggcggcatcgaggtcgtatggacgaacaccccgacgaaatgggacaacagtt

SEQ ID NO: 18 - DNA, 89 bp, synthetic construct
  source 1..89: mol_type = other DNA
  misc_feature 1..89: note = Mutant target
ctcgtatggcaccggaaccggtaaggacgcgatcaccctcggcatcgaggtcgtatggacgaacaccccgacgaaatgggacaacagtt

SEQ ID NO: 19 - DNA, 56 bp, synthetic construct
  source 1..56: mol_type = other DNA
  misc_feature 1..56: note = Mutant target
caccggaaccggtaaggacgtgatcaccaccggcatcgaggtcgtatggacgaaca

SEQ ID NO: 20 - DNA, 56 bp, synthetic construct
  source 1..56: mol_type = other DNA
  misc_feature 1..56: note = Mutant target
caccggaaccggtaaggacgcgatcaccaccgacatcgaggtcgtatggacgaaca

SEQ ID NO: 21 - DNA, 56 bp, synthetic construct
  source 1..56: mol_type = other DNA
  misc_feature 1..56: note = Mutant target
caccggaaccggtaaggacgtgatcaccaccgacatcgaggtcgtatggacgaaca

SEQ ID NO: 22 - DNA, 56 bp, synthetic construct
  source 1..56: mol_type = other DNA
  misc_feature 1..56: note = its a L-DNA
  misc_feature 1..56: note = 5' is conjugated to "TXR", it refers to a Texas Red moiety
  misc_feature 1..56: note = Wild-type drug-susceptible comparator forward strand with end-labeling (L-DNA)
caccggaaccggtaaggacgcgatcaccagcggcatcgaggtcgtatggacgaaca

SEQ ID NO: 23 - DNA, 61 bp, synthetic construct
  source 1..61: mol_type = other DNA
  misc_feature 1..61: note = its a L-DNA
  misc_feature 1..61: note = 3' is conjugated to "BHQ2", it refers to a Black Hole Quencher
  misc_feature 1..61: note = Wild-type drug-susceptible comparator reverse strand with end-labeling (L-DNA)
tgttcgtccatacgacctcgatgccgctggtgatcgcgtccttaccggttccggtgccata

SEQ ID NO: 24 - DNA, 17 bp, synthetic construct
  source 1..17: mol_type = other DNA
  misc_feature 1..17: note = forward primer
caccggaaccggtaagg

SEQ ID NO: 25 - DNA, 19 bp, synthetic construct
  source 1..19: mol_type = other DNA
  misc_feature 1..19: note = reverse primer
tgttcgtccatacgacctc

SEQ ID NO: 26 - DNA, 56 bp, synthetic construct; identical to SEQ ID NO: 22
  source 1..56: mol_type = other DNA
  misc_feature 1..56: note = Wild-type drug-susceptible amplicon forward strand

SEQ ID NO: 27 - DNA, 56 bp, synthetic construct
  source 1..56: mol_type = other DNA
  misc_feature 1..56: note = Wild-type drug-susceptible amplicon reverse strand
tgttcgtccatacgacctcgatgccgctggtgatcgcgtccttaccggttccggtg

SEQ ID NO: 28 - DNA, 56 bp, synthetic construct; identical to SEQ ID NO: 22
  source 1..56: mol_type = other DNA
  misc_feature 1..56: note = its a L-DNA
  misc_feature 1..56: note = Wild-type drug-susceptible internal comparator forward strand with end-labeling (L-DNA)

SEQ ID NO: 29 - DNA, 56 bp, synthetic construct; identical to SEQ ID NO: 27
  source 1..56: mol_type = other DNA
  misc_feature 1..56: note = its a L-DNA
  misc_feature 1..56: note = Wild-type drug-susceptible internal comparator reverse strand with end-labeling (L-DNA)

SEQ ID NO: 30 - DNA, 19 bp, synthetic construct
  source 1..19: mol_type = other DNA
  misc_feature 1..19: note = SMASH assay rpoB forward primer (66 base-pair amplicon)
gatgtgcccgatcgaaacc

SEQ ID NO: 31 - DNA, 17 bp, synthetic construct
  source 1..17: mol_type = other DNA
  misc_feature 1..17: note = SMASH assay rpoB reverse primer (66 base-pair amplicon)
gcgtacaccgacagcga

SEQ ID NO: 32 - DNA, 30 bp, synthetic construct
  source 1..30: mol_type = other DNA
  misc_feature 1..30: note = SMASH assay rifampicin-susceptible melt probe with 3' C3 spacer to block PCR extension
  misc_feature 1..30: note = 3' is conjugated to "3SpC3", it refers to a 3' C3 Spacer modification
gccgatcagaccgatgttgggcccctcagg

SEQ ID NO: 33 - DNA, 30 bp, synthetic construct; identical to SEQ ID NO: 32
  source 1..30: mol_type = other DNA
  misc_feature 1..30: note = its a L-DNA
  misc_feature 1..30: note = 5' is conjugated to "TXR", it refers to a Texas Red moiety
  misc_feature 1..30: note = SMASH assay rifampicin-susceptible L-DNA comparator forward strand with 5' Texas Red-X

SEQ ID NO: 34 - DNA, 30 bp, synthetic construct
  source 1..30: mol_type = other DNA
  misc_feature 1..30: note = its a L-DNA
  misc_feature 1..30: note = SMASH assay rifampicin-susceptible L-DNA comparator reverse complement strand with no modification
cctgaggggcccaacatcggtctgatcggc

SEQ ID NO: 35 - DNA, 19 bp, synthetic construct
  source 1..19: mol_type = other DNA
  misc_feature 1..19: note = Andre I491F assay forward primer (301 base-pair amplicon)
tggagtacgtgccctcgtc

SEQ ID NO: 36 - DNA, 18 bp, synthetic construct
  source 1..18: mol_type = other DNA
  misc_feature 1..18: note = Andre I491F assay forward primer including nucleotide 1471 of rpoB codon 471 (70 base-pair amplicon)
ggcccaacatcggtctga

SEQ ID NO: 37 - DNA, 19 bp, synthetic construct
  source 1..19: mol_type = other DNA
  misc_feature 1..19: note = Andre assay reverse primer (301 or 70 base-pair amplicons)
gtggccaccgacaccatct

SEQ ID NO: 38 - DNA, 327 bp, synthetic construct
  source 1..327: mol_type = other DNA
  misc_feature 1..327: note = Wild-type rifampicin-susceptible target
gatgtgcccgatcgaaacccctgaggggcccaacatcggtctgatcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccac

SEQ ID NO: 39 - DNA, 327 bp, synthetic construct
  source 1..327: mol_type = other DNA
  misc_feature 1..327: note = I491F mutant target
gatgtgcccgatcgaaacccctgaggggcccaacatcggtctgttcggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccac

SEQ ID NO: 40 - DNA, 327 bp, synthetic construct
  source 1..327: mol_type = other DNA
  misc_feature 1..327: note = I491N mutant target
gatgtgcccgatcgaaacccctgaggggcccaacatcggtctgaacggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccac

SEQ ID NO: 41 - DNA, 327 bp, synthetic construct
  source 1..327: mol_type = other DNA
  misc_feature 1..327: note = I491M mutant target
gatgtgcccgatcgaaacccctgaggggcccaacatcggtctgataggctcgctgtcggtgtacgcgcgggtcaacccgttcgggttcatcgaaacgccgtaccgcaaggtggtcgacggcgtggttagcgacgagatcgtgtacctgaccgccgacgaggaggaccgccacgtggtggcacaggccaattcgccgatcgatgcggacggtcgcttcgtcgagccgcgcgtgctggtccgccgcaaggcgggcgaggtggagtacgtgccctcgtctgaggtggactacatggacgtctcgccccgccagatggtgtcggtggccac

SEQ ID NO: 42 - DNA, 27 bp, synthetic construct
  source 1..27: mol_type = other DNA
  misc_feature 1..27: note = Set A susceptible melt probe mimic as reverse complement of wild-type rpoB (27 bases in length)
gatcagaccgatgttgggcccctcagg

SEQ ID NO: 43 - DNA, 27 bp, synthetic construct
  source 1..27: mol_type = other DNA
  misc_feature 1..27: note = Set A asymmetric PCR product mimic as wild-type rpoB (27 bases in length)
cctgaggggcccaacatcggtctgatc

SEQ ID NO: 44 - DNA, 27 bp, synthetic construct
  source 1..27: mol_type = other DNA
  misc_feature 1..27: note = Set A asymmetric PCR product mimic as rpoB I491F variant (27 bases in length)
cctgaggggcccaacatcggtctgttc

SEQ ID NO: 45 - DNA, 28 bp, synthetic construct
  source 1..28: mol_type = other DNA
  misc_feature 1..28: note = Set B susceptible melt probe mimic as reverse complement of wild-type rpoB (28 bases in length)
cgatcagaccgatgttgggcccctcagg

SEQ ID NO: 46 - DNA, 28 bp, synthetic construct
  source 1..28: mol_type = other DNA
  misc_feature 1..28: note = Set B asymmetric PCR product mimic as wild-type rpoB (28 bases in length)
cctgaggggcccaacatcggtctgatcg

SEQ ID NO: 47 - DNA, 28 bp, synthetic construct
  source 1..28: mol_type = other DNA
  misc_feature 1..28: note = Set B asymmetric PCR product mimic as rpoB I491F variant (28 bases in length)
cctgaggggcccaacatcggtctgttcg

SEQ ID NO: 48 - DNA, 29 bp, synthetic construct
  source 1..29: mol_type = other DNA
  misc_feature 1..29: note = Set C susceptible melt probe mimic as reverse complement of wild-type rpoB (29 bases in length)
ccgatcagaccgatgttgggcccctcagg

SEQ ID NO: 49 - DNA, 29 bp, synthetic construct
  source 1..29: mol_type = other DNA
  misc_feature 1..29: note = Set C asymmetric PCR product mimic as wild-type rpoB (29 bases in length)
cctgaggggcccaacatcggtctgatcgg

SEQ ID NO: 50 - DNA, 29 bp, synthetic construct
  source 1..29: mol_type = other DNA
  misc_feature 1..29: note = Set C asymmetric PCR product mimic as rpoB I491F variant (29 bases in length)
cctgaggggcccaacatcggtctgttcgg

SEQ ID NO: 51 - DNA, 30 bp, synthetic construct; identical to SEQ ID NO: 32
  source 1..30: mol_type = other DNA
  misc_feature 1..30: note = Set D susceptible melt probe mimic as reverse complement of wild-type rpoB (30 bases in length)

SEQ ID NO: 52 - DNA, 30 bp, synthetic construct; identical to SEQ ID NO: 34
  source 1..30: mol_type = other DNA
  misc_feature 1..30: note = Set D asymmetric PCR product mimic as wild-type rpoB (30 bases in length)

SEQ ID NO: 53 - DNA, 30 bp, synthetic construct
  source 1..30: mol_type = other DNA
  misc_feature 1..30: note = Set D asymmetric PCR product mimic as rpoB I491F variant (30 bases in length)
cctgaggggcccaacatcggtctgttcggc

(19) United States
(12) Patent Application Publication
(10) Pub. No.: US 2025/0369042 A1
(43) Pub. Date: Dec. 4, 2025

(54) COMPARATIVE TEMPLATING FOR DIRECT SEQUENCE VARIATION DETECTION

(71) Applicant: VANDERBILT UNIVERSITY, Nashville, TN (US)

(72) Inventors: Frederick R. HASELTON, Nashville, TN (US); Nicholas A. SPURLOCK, Nashville, TN (US); Nicole A. Malofsky, Nashville, TN (US)

(21) Appl. No.: 19/223,422

(22) Filed: May 30, 2025

Related U.S. Application Data
(60) Provisional application No. 63/654,462, filed on May 31, 2024.

(51) Int. Cl. C12Q 1/6869; C12Q 1/6806; C12Q 1/689

(52) U.S. Cl. CPC C12Q 1/6869; C12Q 1/6806; C12Q 1/689; C12Q 2600/156; C12Q 2600/166

(57) ABSTRACT

Variation in a single nucleotide of a target or template nucleic acid may be significant in many ways. For infectious disease, many drug resistance mutations are known and a simple means to confine their absence would more quickly match a patient with an infection with the most effective treatment. This disclosure provides for a simple, low resource strategy allowing the detection of single nucleotide variation using only low-cost diagnostic methods already available in many locations. A key to this approach is the use of left-handed DNA (L-DNA) as a comparator molecule for D-DNA targets. L-DNA provides numerous additional advantages such as low cost and stability.

### CROSS REFERENCE TO RELATED APPLICATIONS

This application claims the benefit of U.S. Provisional Patent Application 63/654,462, filed May 31, 2024, the disclosure of which is hereby incorporated by reference in its entirety.

### STATEMENT REGARDING FEDERALLY FUNDED RESEARCH

This invention was made with government support under Grant Nos. AI152497, AI157827, and AI135937, awarded by the National Institutes of Health. The government has certain rights in the invention.

### FIELD OF THE INVENTION

The present disclosure relates generally to fields of molecular biology, nucleic acid chemistry, manufacturing and diagnostics. More particularly, the disclosure relates to the use of L-DNA molecules to identify sequence differences in nucleic acid amplification products.

### INCORPORATION BY REFERENCE

The contents of the xml file named “10644-210US1-ST26” which was created on May 28, 2025, and is 74.2 KB in size, are hereby incorporated by reference in their entirety.

### BACKGROUND OF THE INVENTION

In regions with large populations of people living with infectious disease the lack of simple and cost-effective methods to detect and characterize such infections continues to impede treatment management and the spread of infection. Failure to effectively treat highly infectious diseases leads to more widespread infection. Examples of this are identification of drug-resistant tuberculosis (TB) and HIV.

Melt analysis is one method used to detect differences between a drug-susceptible wild-type sequence and a drug-resistant sequence that contains only a single base difference. Although melt analysis is very sensitive to sequence variation, it requires a carefully calibrated PCR instrument. A less complex, but robust and sensitive reagent design, would make melt analysis more available by enabling implementation with simpler PCR instrumentation.

As such, there remains a significant unmet need for reagent-based methods for detecting nucleic acid sequence variation.

### SUMMARY OF THE INVENTION

The methods described herein enable new approaches for the sensitive detection of sequence variation, for example including but not limited to a single base pair change between an unknown DNA molecule and a corresponding reference DNA molecule, using a simple reagent-based approach that can be performed with calibrated and uncalibrated standard PCR instrumentation alike.

Melt analysis is based on the observation that any given double-stranded DNA sequence dissociates at a characteristic melt temperature (Tm). This property is used to compare the melt temperature of an unknown PCR product to the characteristic melt temperature of the known drug-susceptible wild-type sequence. Any shift from the wild-type melt temperature implies that the unknown test sample contains one or more single nucleotide polymorphisms (SNPs) relative to the reference DNA molecule. Melt analysis utilizes the temperature-control capabilities often available in real-time PCR instrumentation. For example, certain carefully calibrated real-time instruments offer high resolution melt (HRM) capabilities by including a temperature calibration feature. The requirement for instrument calibration to enable the comparison of two or more samples is a major source of complexity in these approaches.

This disclosure describes an approach to accurately and sensitively detect sequence changes by comparing the PCR amplicon product to a known enantiomeric left-helical (L)-DNA equivalent, identical with respect to length and sequence, that is added to the same sample as a standard melt comparator. Both double-stranded L-DNA additive and double-stranded D-DNA PCR product in the same reaction are affected by factors that influence hybridization in the same way. Therefore, given that the melt characteristics of the L-DNA additive and the D-DNA from the PCR amplicon are identical, any difference in melt characteristics observed between the L-DNA and the unknown PCR product is directly attributable to a characteristic change in PCR product sequence.

Features inherent to L-DNA enable the methods described herein. For example, L-DNA does not interfere with or participate in PCR reactions and does not interact with normal biological processes.

The methods disclosed herein achieved comparable classification to existing methods which require carefully calibrated PCR instruments, while relying only on within-sample melt differences between L-DNA and the unknown PCR product. The L-DNA-based methods described herein (e.g., LHRM analysis of katG in Example 1) classified PCR products as drug-susceptible or not drug-susceptible based on identification of multiple and even single base mutations, performing at 77.8% sensitivity and 98.7% specificity (see, e.g., Example 1 herein). By comparison, a state-of-the-art calibrated instrument and multiple sample classification analysis using existing high resolution melt analysis (HRM) methods performed at 66.7% sensitivity and 98.8% specificity.

In one aspect, provided herein is a method comprising providing a set of PCR primers designed to flank a region of DNA that is known to have the potential for sequence variation. The expected unaltered sequence, designated “wild-type” in some descriptions, is then synthesized in L-DNA form with the same length and sequence as that of the wild-type region. The L-DNA form of DNA contains enantiomeric bases that are not found in nature, but exhibit identical properties to the D-DNA bases. The L-DNA wild-type then has, by design, all of the physical, chemical, and melting properties of its D-DNA counterpart, but the L-DNA crucially does not interact with the D-DNA present in the same solution. As described herein, these properties enable the novel use of L-DNA templates as additives to a PCR reaction mix to serve as an exact wild-type comparator during melt analysis for the identification of nucleic acid variation between an unknown DNA molecule and a reference DNA molecule.

In some aspects, sequence variation can be detected in cases where the sequences of the unknown sample and the L-DNA are not identical, but the concentration of L-DNA double strands have been set in advance to act as an effective comparator reagent.

In order to perform the comparison between the unknown PCR amplicon and the L-DNA template, a means to independently detect the melt properties of each is required. As described herein, two methods have been identified to do this. Traditional melt analysis is performed using intercalating dyes that fluoresce much more brightly when bound or intercalated between two strands of hybridized DNA. Based on preliminary studies, all known intercalating dyes intercalate into D-DNA and L-DNA equally. For example, SYBR® Green intercalates and can be used to detect the separation of two complementary strands as a function of temperature for D-DNA, and also for L-DNA as well.

In some embodiments, the L-DNA-based methods as described herein are alternatively referred to as an “L-DNA-based high resolution melt” approach or “LHRM” approach.

In one aspect, provided herein is a method comprising template L-DNA which is added to a PCR reaction in low concentration along with an intercalator, including but not limited to, e.g., SYBR® green, and an initial melt analysis curve is obtained. Importantly, before the PCR reaction has been started the reaction does not contain any detectable double stranded amplicon product so this initial melt analysis only reflects the characteristics of the L-DNA wild type template. After the PCR reaction has been completed, the PCR reaction contains typically more than 10¹² copies of amplicon in D-DNA form. A second melt curve is then performed and compared to the initial melt curve obtained with the low concentration of the L-DNA wild-type template.

The second method compares the intercalating dye to a fluorescent probe on one strand of the L-DNA duplex and a quencher on the complementary strand to do a comparison simultaneously in two PCR fluorescence channels. In this approach, the separation of the labeled strands shows a decrease in the fluorescence signals associated with each of the enantiomeric structures by comparing the melt obtained with the intercalating dye to that obtained with the L-DNA end labeling. For example, the L-DNA wild-type template is labeled with Texas Red dye on one strand and a quencher on the opposite strand and SYBR® green is added as the intercalating dye. This allows comparison between the Texas Red channel for L-DNA alone and the SYBR® green channel for all double-stranded structures. By selecting and controlling the concentration of the two dyes, the L-DNA end-label signal can be used to determine the melt characteristics of each structure.

In another aspect, provided herein is a method comprising several L-DNA “standard” structures with known melt characteristics that span the unknown sample melt characteristics. In this method, the melt characteristics are determined in reference to L-DNA standards. This could be implemented by spiking the standards into the reaction before the PCR reaction. Since these structures are made from L-DNA they do not interact with or interfere with the enzymes or other biological structures present in the PCR reaction. In this design, the L-DNA structures produce characteristic melt peaks obtained by existing methods and this scale is then overlaid with the melt peak of the unknown to determine the melt characteristics of the unknown. This is completely analogous to how “standards” are included within gel electrophoresis of DNA structures. In gels, the standards with known DNA characteristics (usually length) produce a pattern in a separate lane of the gel and this pattern of knowns is compared with the unknown.

In an alternative aspect, provided herein is a method using “melt” probes, wherein asymmetric PCR is used to produce an excess of one strand and “probes” with complementarity to this strand are melted from this strand at the end of PCR amplification, wherein shifts in the melt probe properties between a known sample and an unknown sample are used to identify sequence variation. Short double-stranded L-DNA that match the melt characteristics of the D-DNA components are again used as known standards for comparison within a single well without the need for comparison between samples.

Reassuringly, the “melt” probe-based methods disclosed herein using asymmetric PCR achieved 100% sensitivity and 100% specificity in classifying synthetic unknown samples as drug (e.g., rifampicin) susceptible at codon 491 of the rpoB gene of Mycobacterium tuberculosis (see, e.g., Example 2 herein). By comparison, using a state-of-the-art calibrated instrument and multiple sample classification analysis, existing high resolution melt analysis (HRM) methods performed at 66.7% sensitivity and 98.8% specificity.

In another embodiment, provided herein is a kit comprising a double stranded L-DNA and at least one detectable moiety. In some embodiments, the L-DNA has the same sequence as a naturally occurring D-DNA sequence. In some embodiments, the L-DNA has less than about 10% base differences as compared to a naturally occurring D-DNA sequence, or the L-DNA has less than about 90%, less than about 80%, less than about 70%, less than about 60%, less than about 50%, less than about 40%, less than about 30% or less than about 20% identity relative to a naturally occurring D-DNA sequence. In some embodiments, the kit further comprises standard double-stranded sequences L-DNA and/or D-DNA sequence that have known melt temperatures and/or elapsed melt times. In some embodiments, the L-DNA comprise partial sequence pairs for molecular beacon detection.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows a multi-sample comparison of melt temperature (Tm) obtained from standard HRM of eleven representative samples containing either wild-type, one of nine variants, or no template control (NTC) PCR products. Dashed vertical lines indicate the Tm for each sample on the horizontal axis.

FIG. 2 shows PCR product melt temperatures of samples analyzed by standard HRM across wild-type and nine variants. Samples were classified as drug-susceptible or not by comparing sample Tm to the drug-susceptible Tm cutoff range of 82.4° C. and 82.5° C. (indicated by dashed black lines). Each point represents an individual test sample.

FIG. 3 shows PCR product melt temperatures of samples containing an L-DNA comparator in every sample but analyzed by standard HRM across wild-type and nine variants. Samples were classified as drug-susceptible or not by comparing sample Tm to the drug-susceptible Tm cutoff range of 82.4° C. and 82.5° C. (indicated by dashed black lines).

FIG. 4 shows within-sample comparison of tm obtained from LHRM for eleven representative samples containing both wild-type L-DNA and either wild-type, variant, or NTC PCR products. Dashed vertical lines indicate the two tm's within each sample on the horizontal axis.

FIG. 5 shows within-sample tm differences of samples containing an L-DNA comparator in every sample and analyzed by LHRM across wild-type and nine variants. Samples were classified as drug-susceptible or not by comparing sample tm difference to the drug-susceptible classification criteria of tm difference=0 (indicated by dashed black lines).

FIGS. 6A-6C show Melt curve derivatives for LHRM samples with 1:1 double-stranded L-DNA. As total L-DNA copy number decreased, both fluorophore-quencher signal and intercalator signal decreased in magnitude. End-labeled strands retained tm identification even when strand copy count decreased. Dashed lines indicate L-DNA tm measured by fluorophore-quencher signal.

FIG. 6A shows melt curve derivatives for LHRM samples with 1:1 double-stranded L-DNA at 4×10¹¹ copies per strand (forward and reverse) per reaction.

FIG. 6B shows melt curve derivatives for LHRM samples with 1:1 double-stranded L-DNA at 2×10¹¹ copies per strand (forward and reverse) per reaction.

FIG. 6C shows show melt curve derivatives for LHRM samples with 1:1 double-stranded L-DNA at 1×10¹¹ copies per strand (forward and reverse) per reaction.

FIG. 7A shows Representative wild-type D-DNA PCR product and internal comparator L-DNA derivative melt plots analyzed by LHRM analysis containing double-stranded L-DNA at 1:1, 1:2, and 1:3 ratio of forward to reverse L-DNA strands with 1×10¹¹ forward strand copies and 1×10¹¹, 2×10¹¹, and 3×10¹¹ reverse strand copies per reaction, respectively. Dashed lines indicate D-DNA PCR product tm and L-DNA tm.

FIG. 7B shows that there is a positive linear relationship between number of L-DNA reverse strand copies and L-DNA tm measured via fluorophore-quencher signal. This relationship suggested that an L-DNA forward to reverse strand ratio of 1:2.79 (1×10¹¹ forward strand copies and 2.79×10¹¹ reverse strand copies per reaction) would match drug-susceptible L-DNA tm to the drug-susceptible D-DNA PCR product average tm of 695 sec.

FIG. 8 shows standard HRM relies on comparison of temperature-based melt characteristics between two samples. Dashed lines indicate Tm's of drug-susceptible and not drug-susceptible PCR products.

FIG. 9 shows LHRM compares time-based melt characteristics within one sample using a drug-susceptible L-DNA internal comparator. Dashed lines indicate PCR product tm and drug-susceptible L-DNA tm.

FIGS. 10A-10B show Standard HRM PCR product Tm's.

FIG. 10A shows Standard HRM PCR product Tm of wild-type katG samples across the top left and top right quadrants, respectively, of a 96-well plate.

FIG. 10B shows standard HRM PCR product Tm of S315T samples across the top left and top right quadrants, respectively, of a 96-well plate.

FIG. 11 shows a comparison of LHRM and standard HRM analysis strategies. Direct linear relationship between LHRM analysis (tm difference) and standard HRM analysis (Tm) of samples containing an L-DNA comparator in every sample across wild-type and nine variant sample types. Each point represents an individual test sample analyzed using two different analysis strategies.

FIG. 12 shows a heatmap of the Youden J Statistic across upper (positive) and lower (negative) bound drug-susceptible Tm cutoff values (in ° C.) from samples without L-DNA and analyzed using standard HRM analysis.

FIG. 13 shows PCR product melt temperatures of samples without L-DNA and analyzed by standard HRM across wild-type and nine variants (n=3 trials in triplicate per sample type). Samples were classified as drug-susceptible or not by comparing sample Tm to the drug-susceptible Tm cutoff range of 82.4° C. and 82.8° C. (indicated by dashed black lines). Each point represents an individual test sample.

FIG. 14 shows a heatmap of the Youden J Statistic across upper (positive) and lower (negative) bound drug-susceptible Tm cutoff values (in ° C.) from samples containing L-DNA but analyzed using standard HRM analysis.

FIG. 15 shows PCR product melt temperatures of samples containing L-DNA but analyzed by standard HRM analysis, including wild-type and nine variants (n=3 trials in triplicate per sample type, except variant S315T+G316D+A312V of one trial with a single replicate due to Cq exclusion). Samples were classified as drug-susceptible or not by comparing sample Tm to the drug-susceptible Tm cutoff range of 82.4° C. to 82.8° C. (indicated by dashed black lines). Each point represents an individual test sample.

FIG. 16 shows a heatmap of the Youden J Statistic across upper (positive) and lower (negative) bound drug-susceptible tm difference cutoff values (in seconds) from samples containing L-DNA and analyzed using LHRM analysis.

FIG. 17 shows within-sample tm differences of samples containing L-DNA and analyzed by LHRM analysis, including wild-type and nine variants (n=3 trials in triplicate per sample type, except variant S315T+G316D+A312V of one trial with a single replicate due to Cq exclusion). Samples were classified as drug-susceptible or not by comparing sample tm difference to the drug-susceptible tm difference cutoff range of −8 sec to 8 sec (indicated by dashed black lines). Each point represents an individual test sample.

FIG. 18 shows representative PCR amplification curves of samples without L-DNA across wild-type (WT) katG, nine katG variants, and NTC sample types.

FIG. 19 shows representative PCR amplification curves of samples containing L-DNA across wild-type (WT) katG, nine katG variants, and NTC sample types.

FIG. 20 shows a single sample melt analysis.

FIGS. 21A-21B show performance of the SMASH assay using the QuantStudio™ 5 real-time PCR instrument.

FIG. 21A shows within-sample Tm differences of SMASH samples across wild-type and variants I491F, I491N, and I491M. Each technical replicate is represented by a single dot. Average Tm difference±standard deviation per sample type is indicated across n=6 trials in triplicate per sample type. Statistical significance is indicated by solid black lines with an asterisk (p<0.0001).

FIG. 21B shows within-sample Tm comparison for four representative samples containing susceptible L-DNA and susceptible melt probe duplexed to wild-type or variant asymmetric PCR product. Dashed vertical lines indicate the Tms within each sample for L-DNA and the probe-product duplex on the horizontal axis.

FIGS. 22A-22B show performance of the SMASH assay using the Rotor-Gene® Q real-time PCR instrument.

FIG. 22A shows within-sample Tm differences of SMASH samples across wild-type and variants I491F, 1491N, and I491M. Each technical replicate is represented by a single dot. Average Tm difference±standard deviation per sample type is indicated across n=6 trials in triplicate per sample type. Statistical significance is indicated by solid black lines with an asterisk (p<0.0001).

FIG. 22B shows within-sample Tm comparison for four representative samples containing susceptible L-DNA and susceptible melt probe duplexed to wild-type or variant asymmetric PCR product. Dashed vertical lines indicate the Tms within each sample for L-DNA and the probe-product duplex on the horizontal axis.

FIGS. 23A-23B show performance of the Andre 1491F assay using the QuantStudio™ 5 real-time PCR instrument.

FIG. 23A shows Tm differences between each sample's PCR product Tm and the sample set's global average wild-type Tm across wild-type and variants 1491F, 1491N, and I491M. Each technical replicate is represented by a single dot. Average Tm difference±standard deviation per sample type is indicated across n=3 trials in triplicate per sample type. Statistical significance is indicated by a solid black line with an asterisk (p<0.0001).

FIG. 23B shows multi-sample Tm comparison for five representative samples plotted together containing either wild-type, one of three variants, or no template control (NTC). Dashed vertical lines indicate the Tm for each sample on the horizontal axis.

FIG. 24 shows a comparison of melt differences between susceptible melt probe mimics duplexed to either wild-type rpoB or I491F variant using the QuantStudio™ 5. Four probe sets (A-D) were tested with corresponding product strands of increasing length and offset (Table 14) of rpoB nucleotide 1471. Tm differences were calculated between each sample's duplex Tm and the average Tm of duplexed melt probe to wild-type for that particular set. Consistent with duplex destabilization principles, increasing the mismatch offset (across Set A to Set D) led to larger melt shifts between the probe—I491F duplex and the probe—wild-type duplex.

FIG. 25 shows there is a positive logarithmic relationship (dashed black line) between susceptible melt probe concentration per reaction and Tm of duplexed melt probe to wild-type asymmetric PCR product (measured via intercalation signal, n=1 trial in triplicate per concentration). This relationship suggested that a melt probe concentration of 3.665×10¹² copies per reaction (0.487×) would match the Tm of duplexed probe and wild-type product to the susceptible L-DNA average Tm of 75.30° C. (n=1 trial in triplicate across wild-type, variants I491F/N/M, and NTC sample types).

FIG. 26 shows representative PCR amplification curves of samples tested using the SMASH assay on the QuantStudio™ 5 across wild-type rpoB, three rpoB variants, and NTC sample types.

FIG. 27 shows representative PCR amplification curves of samples tested using the SMASH assay on the Rotor-Gene® Q across wild-type rpoB, three rpoB variants, and NTC sample types.

FIG. 28 shows representative PCR amplification curves of samples tested using the André I491F assay on the QuantStudio™ 5 across wild-type rpoB, three rpoB variants, and NTC sample types.

FIG. 29 shows post-PCR melt curves of representative no template control (NTC) samples produced by the SMASH assay using the QuantStudio™ 5 (left) and Rotor-Gene® Q (right) real-time PCR instruments. Samples contained double-stranded susceptible L-DNA and unbound susceptible melt probe (because no target was present in NTC samples). For each instrument, L-DNA had nearly identical Tm's measured using iFRET signal on the orange optical channel and using intercalation signal on the green optical channel within a single-sample. Both instruments demonstrated higher magnitude of fluorescent outputs when the melt curves were produced using iFRET versus intercalation alone. Dashed vertical lines indicate the Tm's within each sample for L-DNA using iFRET and intercalation alone on the horizontal axis.

FIGS. 30A-30B show Performance of the SMASH assay without incorporating the L-DNA melt data and instead using multi-sample Tm difference comparison for susceptibility classification.

FIG. 30A shows The QuantStudio™ 5 sample set assessed for Tm differences between each sample's probe-product duplex Tm and the sample set's global average Tm of probe bound to wild-type product across wild-type and variants I491F, I491N, and I491M. Samples were classified as drug-susceptible when sample Tm difference was less than the drug-susceptible cutoff of 1.43° C. (indicated by dashed black line and determined by ROC cutoff analysis in Table 16). Each technical replicate is represented by a single dot. Average Tm difference±standard deviation per sample type is indicated across n=6 trials in triplicate per sample type. Statistical significance is indicated by solid black lines with asterisks (p<0.0001).

FIG. 30B shows The Rotor-Gene® Q sample set assessed for Tm differences between each sample's probe-product duplex Tm and the sample set's global average Tm of probe bound to wild-type product across wild-type and variants I491F, I491N, and I491M. Samples were classified as drug-susceptible when sample Tm difference was less than the drug-susceptible cutoff of 1.43° C. (indicated by dashed black line and determined by ROC cutoff analysis in Table 16). Each technical replicate is represented by a single dot. Average Tm difference±standard deviation per sample type is indicated across n=6 trials in triplicate per sample type. Statistical significance is indicated by solid black lines with an asterisk (p<0.0001).

FIG. 31 shows single-sample melt-based screening.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

Unless defined otherwise, all technical and scientific terms used herein have the same meaning as commonly understood to one of ordinary skill in the art to which this disclosure belongs.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “asymmetric PCR” as used herein refers to a well-known PCR method in the art, which produces large amount of single strand DNA with a pair of primers of unequal amount in the PCR. The pair of primers comprises a non-limiting primer (i.e., higher concentration primer) and a limiting primer (i.e., low concentration primer). In asymmetric PCR, the early stage of the PCR reaction predominantly produces double strand DNA. Following depletion of the limiting primer, the PCR led by the non-limiting primer produces large amounts of single strand DNA.

As used herein, the term “complementary” refers to bases of one nucleic acid molecule forming a hydrogen bond to the corresponding bases of another nucleic acid molecule. Normally, the base adenine (A) is complementary to thymidine (T) and uracil (U), while cytosine (C) is complementary to guanine (G).

The terms “emission” or “emission signal” as used herein refer to the light of a wavelength generated from a fluorophore after the fluorophore absorbs light at its excitation wavelength(s).

The terms “excitation” or “excitation signal” as used herein refer to the light of a particular wavelength necessary to excite a fluorophore to a state such that the fluorophore will emit a different wavelength of light.

The term “fluorophore” as used herein refers to a chemical compound, which when excited by exposure to a particular stimulus such as a defined wavelength of light, emits light (fluoresces), for example at a different wavelength (such as a longer wavelength of light). A detailed description of alternative fluorophores are provided herein.

The term “Fluorescence Resonance Energy Transfer” or “FRET” as used herein refers to a spectroscopic process by which energy is passed between an initially excited donor to an acceptor molecule separated by 10-100 Å.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. On occasion double-stranded DNA will be referred to “duplex DNA” or “dsDNA”. As referred to herein, D-DNA is used to describe right-handed DNA helix structure. L-DNA is used to describe left-handed DNA helix structure.

Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “primer” as used herein refers to a nucleic acid molecule, such as a DNA oligonucleotide, for example sequences of at least 15 nucleotides, which can be annealed to a complementary target nucleic acid molecule by nucleic acid hybridization to form a hybrid complex between the primer and the target nucleic acid strand. A primer can be extended along the target nucleic acid molecule by a polymerase enzyme such as a PCR technique. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

The term “probe” refers to an isolated nucleic acid capable of hybridizing to a complementary sequence of a target nucleic acid. In some embodiments, a detectable label or reporter molecule is attached to a probe to enable detection of a target nucleic acid.

“Sequence identity” or “identity” in the context of nucleic acid sequences refers to the nucleic acid base residues in two sequences that are the same when aligned for maximum correspondence over a specified comparison window.

The term “percentage of sequence identity” refers to the value determined by comparing two optimally aligned sequences over a comparison window, wherein the portion of the nucleic acid sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base residue occurs in both sequences to yield the number of matched positions, dividing the number of matched positions by the total number of positions in the window of comparison and multiplying the results by 100 to yield the percentage of sequence identity. Useful examples of percent identities include, but are not limited to, 50%, 55%, 60%, 65%, 70%, 75%, 80%, 85%, 90% or 95%, or any percentage from 50% to 100%. Indeed, any sequence identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

A “nucleic acid variation” refers to an unknown nucleic acid sequence of interest that comprises at least one alteration when compared to its non-modified, reference nucleic acid sequence. Such “alterations” include, but are not limited to, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

As discussed above, low resource but highly sensitive assays to detect subtle changes, including single nucleotide substitutions and deletions, are greatly in need.

Melt analysis is a method of detecting sequence variation based on the characteristic melt temperature (i.e., Tm) of a specific sequence. This melt temperature is defined as the reaction temperature at which 50% of the double-stranded DNA is dissociated into single strands. Melt temperatures on a highly calibrated instrument are provided as a feature of the instrument.

Melt analysis can also detect sequence variation based on the characteristic elapsed melt time of a specific sequence. This elapsed melt time is defined as the amount of elapsed time from the start of melt heat cycling up to the time at which 50% of the double-stranded DNA is dissociated into single strands. Elapsed melt time uses the property of standard PCR instruments to have a constant heat ramp source during the melt analysis.

In one aspect, provided herein are single-tube comparator methods based on the inclusion of an internal synthetic comparator with melt characteristics identical to, or having defined differences from, the wild-type PCR product to simplify reagent design and melt analysis measurements. Preliminary data suggest that melt analysis based on this approach has the single-base sensitivity required for the detection of susceptibility to a certain therapeutic, including but not limited to isoniazid (INH) susceptibility (see, e.g., Example 1 herein) or rifampicin (see, e.g., Example 2). In a particular application, the sequence sensitivity of this approach for single nucleotide polymorphisms can be applied to the TB INH resistance-determining region. In some embodiments, the sequence sensitivity of the approaches described herein can be applied to the TB rifampicin resistance determining region, for example including but not limited to rpoB codon 491.

The reagent design, based on L-DNA, makes melt analysis approach possible in resource-constrained settings as an adjunct to PCR testing. Because the method is sequence-based, it is anticipated that it can be easily modified for use in other diagnostic scenarios where knowledge of small changes in a known drug-susceptible sequence impacts the treatment management decision.

L-DNA has a number of features that make it ideal as a comparator for D-DNA sequences. First, L-DNA is biologically inert and resistant to degradation. There are no natural nucleases that target and degrade L-DNA (Urata et al., 1991). Second, L-DNA is easily and inexpensively manufactured in large quantities. L-DNAs are synthesized using the same phosphoramidite chemistry that is used to synthesize DNA oligonucleotides. Third, and perhaps most significantly, L-DNA does not interact with the D-DNA present in the same solution. These properties enable the use of L-DNA templates as additives to a PCR reaction mix to serve as a perfect wild-type comparator during melt analysis.

Collectively, the data shown in this disclosure support the hypothesis that adding L-DNA improves standard high-resolution melt analysis (HRM). In the context of drug susceptibility screening for the TB drug INH, the methods described herein improved the classification of known synthetic variants compared to standard HRM (see, e.g., Example 1). In another example, drug susceptibility screening methods described herein for the TB drug rifampicin led to highly sensitive screening for clinically significant mutations (see, e.g., Example 2).

In alternative aspects, L-DNA-based HRM designs as described herein can be applied to other clinical applications, which may include screening for SNP-induced diseases, disease variants, or drug-resistance by adapting the key design features disclosed herein for other screening applications. These key requirements are, for example, (1) including the same amount of double stranded L-DNA in every sample to provide a signature comparator hybridization event; (2) identifying and using a concentration of the double stranded L-DNA produces sufficient fluorophore-quencher signal for accurate L-DNA melt measurements with little crosstalk contribution detectable in the intercalator channel used for D-DNA melt measurements; (3) matching the melt characteristics of the L-DNA melt comparator to the melt characteristics of the wild-type PCR product, which may include increasing the concentration of the quencher strand of the L-DNA or modifying the sequence of the L-DNA.

With the use of these features, L-DNA-based HRM methods as described herein improves the classification of PCR melt products by quantifying within sample differences between the constant L-DNA comparator and an unknown PCR product.

These and other aspects of the disclosure are described in detail below.

### L-DNA

L-DNA is the enantiomer of the natural D-DNA. L-Deoxyribose, the sugar backbone of L-DNA, is mirror of natural D-deoxyribose and has not been found in nature, despite the fact that other L-sugars (e.g., L-arabinose, L-lyxose, L-galactose, L-sorbose, and L-xylulose) do exist in nature. L-DNA can hybridize with complementary L-DNA sequences via classical Watson-Crick base-pairing, forming an L-DNA duplex much like a D-DNA duplex, except that the L-DNA duplex is a left-handed B-helix, a mirror structure of the right-handed 8-helix formed by D-DNA. Importantly, however, L-DNA does not hybridize with the complementary sequence on D-DNA backbones or D-RNA at physiological temperatures.

Given that L-DNA is the enantiomer of D-DNA, the chemical reactivity of L-DNA is identical to D-DNA if the reactant is achiral. The biological reactivity of L-DNA with the chiral proteins and enzymes, however, is completely distinguishable from that of D-DNA. For example, L-DNA is not susceptible to natural DNA-modifying enzymes (e.g., ligases, polymerases and nucleases that can easily modify or degrade natural D-DNA). Taking advantage of these properties, L-DNA has been used to build intracellular nano-sensors as well as aptamers that exhibit enhanced serum stability. L-DNA has also been employed to construct a self-assembled DNA tetrahedral nanostructure showing high cellular uptake. L-DNA has also been adapted to PCR (see Adams et al., 2016) and used as a biostable DNAzyme with achiral metal ions (see Cui et al., 2016). Various aspects and uses of L-DNA is reviewed in Young et al., 2019. L-DNA also has been shown to interact with intercalating dyes just the same as D-DNA.

### L-DNA Synthesis

The first chemical synthesis of L-DNA oligonucleotides was reported by Anderson et al., (1984) who prepared L-dU 18-mer by using the triester approach (Reese, 1978; Narang et al., 1980), while a more recent publication describes synthesis using either a- or β-anomers of L-deoxy-nucleosides and covalently linked to an acridine derivative (Asseline et al., 1991), the contents of which are incorporated herein for all purposes.

### Melt Curves

### Melt Curve Analysis

Melting curve analysis is an assessment of the dissociation characteristics of double-stranded DNA during heating. As the temperature is raised to and above the melt temperature of DNA, the double strand dissociates which leads to a rise in the absorbance of light at 260 nm as well as in hyperchromicity. The temperature at which 50% of the DNA strands are denatured is known as the melting temperature (otherwise referred to as “Tm”, not to be confused with “elapsed melt time” or “tm” also described herein). Measurement of melting temperature can help characterize a PCR product as every dsDNA sequence has a characteristic melting temperature.

For example, the melt temperature information gathered can be used to infer the presence and identity of single-nucleotide polymorphisms (SNP). This is because G-C base pairing have 3 hydrogen bonds between them while A-T base pairs have only 2. DNA with a higher G-C content, whether because of its source (G-C contents: E. coli 0.50, M. luteus 0.72, poly d(AT) 0.00) or, as previously mentioned, because of SNPs, will have a higher melting temperature than DNA with a higher A-T content. Base interactions with neighboring bases also affect the melt temperature.

The melt temperature information also gives vital clues to a molecule's mode of interaction with DNA. Molecules such as intercalators slot in between base pairs and interact through pi stacking. This has a stabilizing effect on DNA's structure which, along with “nearest neighbors” effects, can lead to a rise in its melting temperature. Likewise, increasing salt concentrations helps diffuse negative repulsions between the phosphates in the DNA's backbone. This also leads to an increase in the DNA's melting temperature. Conversely, pH can have a negative effect on the stability of DNA which may lead to a lowering of its melting temperature (Botezatu et al., 2011).

The energy required to break the base-base hydrogen bonding between two strands of DNA is dependent on the length of the DNA strands, GC content, as well as strand complementarity and “nearest neighbors.” By heating a reaction-mixture that contains double-stranded DNA sequences and measuring dissociation against temperature, these attributes can be inferred. Originally, strand dissociation was observed using UV absorbance measurements, but techniques based on fluorescence measurements are now the most common approach.

The temperature-dependent dissociation between two DNA-strands can be measured using a DNA-intercalating fluorophore such as SYBR® green, EvaGreen® or fluorophore-labeled DNA probes. In the case of SYBR® green (which fluoresces 1000-fold more intensely while intercalated in the minor groove of two strands of DNA), the dissociation of the DNA during heating is measurable by the large reduction in fluorescence. Alternatively, juxtapositioned probes (one featuring a fluorophore and the other, a suitable quencher; often referred to as “molecular beacons”) can be used to determine the complementarity of the probe to the target sequence when the strands are separated.

In some embodiments of any method described herein, an intercalating dye is provided that preferentially intercalates within double stranded L-DNA or double stranded D-DNA. In some embodiments, the intercalating dye preferentially intercalates within double stranded L-DNA.

SYBR® Green enabled product differentiation in the LightCycler in 1997. Hybridization probes (or FRET probes) were also demonstrated to provide very specific melting curves from the single-stranded (ss) probe-to-amplicon hybrid. Idaho Technology and Roche have done much to popularize this use on the LightCycler instrument.

Since the late 1990s product analysis via SYBR® Green, other double-strand specific dyes have been developed, and probe-based melting curve analysis has become nearly ubiquitous. The probe-based technique is sensitive enough to detect single-nucleotide polymorphisms (SNP) and can distinguish between homozygous wildtype, heterozygous and homozygous mutant alleles by virtue of the dissociation patterns produced. Without probes, amplicon melting (melting and analysis of the entire PCR product) was not generally successful at finding single base variants through melting profiles. With higher resolution instruments and advanced dyes, amplicon melting analysis of one-base variants is now possible with several commercially available instruments. Such instruments include but are not limited to: Applied Biosystems 7500 Fast System, the 7900HT Fast Real-Time PCR System, Idaho Technology's LightScanner (the first plate-based high resolution melting device), Qiagen's Rotor-Gene instruments, Roche's LightCycler 480 instruments, and Applied Biosystems™ QuantStudio™ 5 real-time PCR thermal cycler (Thermo Fisher Scientific #A28137). Many research and clinical examples exist in the literature that show the use of melting curve analysis to obviate or complement sequencing efforts, and thus reduce costs.

While most real-time PCR machines have the option of melting curve generation and visualization, the level of analysis and software support varies. In general, they do not have high resolution capabilities. High Resolution Melt (known as either Hi-Res Melting, or HRM) is the advancement of this general technology and has begun to offer higher sensitivity for SNP detection within an entire dye-intercalating amplicon. It is less expensive and simpler in design to develop probeless melting curve systems. However, for genotyping applications, where large volumes of samples must be processed, the cost of development may be less important than the total throughput and ease of interpretation, thus favoring sequence probe-based genotyping methods.

In one aspect, the method for calculating and matching melt temperatures of L- and D-DNA strands employs the Van't Hoff equation which relates the change in the equilibrium constant, Keq, of a chemical reaction to the change in temperature, T, given the standard enthalpy change, ΔᵣH^{⊖}, for the process. The subscript r means “reaction” and the superscript ⊖ means “standard”. It was proposed by Dutch chemist Jacobus Henricus van't Hoff in 1884 in his book Etudes de Dynarnique chimique (Studies in Dynamic Chemistry). The Van't Hoff equation has been widely utilized to explore the changes in state functions in a thermodynamic system. The Van't Hoff plot, which is derived from this equation, is especially effective in estimating the change in enthalpy and entropy of a chemical reaction.

### Melt Analysis Detection Formats

In some embodiments, the methods described herein have sufficient amounts of both the L-DNA comparator and the D-DNA unknown. While the user can control L-DNA by simply adding as much as needed, the D-DNA unknown cannot be as easily manipulated. One way to ensure sufficiently high copy number to enable detection during heating that suddenly separates the double strands at a particular temperature is by PCR, which can make numerous copies of the unknown. However, only the primer sites flanking the region of interest are guaranteed to be of a known sequence. In contrast the sequence between the primers may vary as it is copied from the template target sequence. The melt approaches applied here detect whether that unknown internal sequence between the primers is an exact match for the expected sequence.

In order to perform a comparison between an unknown target (e.g., a PCR amplicon) and the L-DNA comparator, a way to independently detect the melt properties of each is required.

In one aspect, provided herein is a traditional melt analysis performed using intercalating dyes that fluoresce much more brightly when bound or intercalated between two strands of hybridized DNA. Based on current studies, all known intercalating dyes intercalate into D-DNA and L-DNA equally. For example, SYBR® green intercalates and can be used to detect the separation of two complementary strands as a function of temperature for D-DNA, but also for L-DNA as well. In this method, the template L-DNA is added to a PCR reaction in low concentration along with a dye (e.g., SYBR® green) and an initial melt analysis curve is obtained. Importantly, before the PCR reaction has been started, the reaction does not contain any detectable double-stranded amplicon product. Thus, this initial melt analysis only reflects the characteristics of the L-DNA wild-type template. After the PCR reaction has been completed, the PCR reaction would typically contain more than 10¹² copies of amplicon in D-DNA form. A second melt curve is then performed and compared to the initial melt curve obtained with the low concentration of the L-DNA wild-type template.

In another aspect, provided herein is a method which compares the intercalating dye to a fluorescent probe on one strand of the L-DNA duplex and a quencher on the complementary strand to do a comparison simultaneously in two PCR fluorescence channels. In some embodiments, the separation of the labeled strands shows a decrease in the fluorescence signals associated with the enantiomeric structures by comparing the melt properties obtained with the intercalating dye to that obtained with the L-DNA end labeling. For example, the L-DNA wild-type template is labelled with Texas Red dye on one strand and a quencher on the opposite strand; then SYBR® green is added as the intercalating dye. This allows comparison between the Texas Red channel for L-DNA alone and the SYBR® green channel for all double-stranded structures. By selecting and controlling the concentration of the L-DNA comparator, the L-DNA end-label signal can be used to determine the melt characteristics of each structure.

In an alternative aspect, provided herein is a method based on asymmetric PCR amplification. In this method the primer ratio is adjusted so that when one primer is exhausted the remaining primer produces an excess of one of the PCR amplicon strands. Changes in the sequence of this excess strand is determined by comparing the melt temperature or elapsed melt time of a targeted D-DNA molecular beacon (added to the reaction) to this excess D-DNA strand in comparison to L-DNA reagents added as equivalent molecular beacon and an analog of the excess D-DNA strand.

In an alternative aspect, provided herein is a method which includes several L-DNA “standard” structures with known melt characteristics that span the unknown nucleic acid sample melt characteristics. In this design, the melt characteristics of the unknown nucleic acid are determined in reference to L-DNA standards. This could be implemented by spiking the L-DNA standards into the reaction before the PCR reaction. Since these structures are made from L-DNA, they do not interact with or interfere with the enzymes or other biological structures present in the PCR reaction. In this design, the L-DNA structures produce known characteristic melt peaks obtained by existing methods that determine the derivative by fluorescence versus temperature. This scale is then overlaid with the melt peak of the unknown nucleic acid to determine the melt characteristics of the unknown. This is analogous to how “standards” are included within gel electrophoresis of DNA structures. In gels, the standards with known DNA characteristics (usually length) produce a pattern in a separate lane of the gel and this pattern of knowns is compared with the unknown nucleic acid.

### Other Techniques and Reagents

### Template Dependent Nucleic Acid Amplification

A number of template dependent processes are available to amplify the sequences present in a given template sample. One of the best-known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the sequence of interest. An excess of deoxynucleoside triphosphates is added to a reaction mixture along with a DNA polymerase, e.g., Taq polymerase. If the sequence of interest is present in a sample, the primers will bind to the sequence of interest and the polymerase will cause the primers to be extended along the sequence of interest by adding on nucleotides. By raising and lowering the temperature of the reaction mixture, the extended primers will dissociate from the sequence of interest to form reaction products, excess primers will bind to the sequence of interest and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present disclosure. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification method is described in GB Application No. 2 202 328, and in PCT Application No. PCT/US89/01025, each of which is incorporated herein by reference in its entirety, may be used in accordance with the present disclosure. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence-based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made folly double stranded by addition of second target specific primer, followed by polymerization. The double-stranded DNA molecules are then multiply transcribed by an RNA polymerase such as 17 or SV6. In an isothermal cyclic reaction, the RNAs are reverse transcribed into single stranded DNA, which is then convelted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present disclosure. The ssRNA is a template for a first primer oligonucleotide, which is elongated by reverse transcriptase (RNA-dependent DNA polymerase). The RNA is then removed from the resulting DNA: RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present disclosure (Wu et al., 1989, incorporated herein by reference in its entirety).

### Intercalating Dyes

In biochemistry, intercalation is the inseltion of molecules between the planar bases of deoxyribonucleic acid (DNA). This process is used as a method for analyzing DNA and it is also the basis of certain kinds of poisoning. There are several ways molecules (in this case, also known as ligands) can interact with DNA. Ligands may interact with DNA by covalently binding, electrostatically binding, or intercalating. Intercalation occurs when ligands of an appropriate size and chemical nature fit themselves in between base pairs of DNA. These ligands are mostly polycyclic, aromatic, and planar, and therefore often make good nucleic acid stains. Intensively studied DNA intercalators include berberine, ethidium bromide, proflavine, daunomycin, doxorubicin, and thalidomide. DNA intercalators are used in chemotherapeutic treatment to inhibit DNA replication in rapidly growing cancer cells. Examples include doxorubicin (adriamycin) and daunorubicin (both of which are used in treatment of Hodgkin's lymphoma), and dactinomycin (used in Wilm's tumor, Ewing's Sarcoma, rhabdomyosarcoma).

Metallointercalators are complexes of a metal cation with polycyclic aromatic ligands. The most commonly used metal ion is ruthenium (II), because its complexes are very slow to decompose in the biological environment. Other metallic cations that have been used include rhodium (III) and iridium (III). Typical ligands attached to the metal ion are dipyridine and terpyridine whose planar structure is ideal for intercalation.

In order for an intercalator to fit between base pairs, the DNA must dynamically open a space between its base pairs by unwinding. The degree of unwinding varies depending on the intercalator; for example, ethidium cation (the ionic form of ethidium bromide found in aqueous solution) unwinds DNA by about 26°, whereas proflavine unwinds it by about 17°. This unwinding causes the base pairs to separate, or “rise”, creating an opening of about 0.34 nm (3.4 Å). This unwinding induces local structural changes to the DNA strand, such as lengthening of the DNA strand or twisting of the base pairs. These structural modifications can lead to functional changes, often to the inhibition of transcription and replication and DNA repair processes, which makes intercalators potent mutagens. For this reason, DNA intercalators are often carcinogenic, such as the exo (but not the endo) 8,9 epoxide of aflatoxin Bl and acridines such as proflavine or quinacrine.

Intercalation as a mechanism of interaction between cationic, planar, polycyclic aromatic systems of the correct size (on the order of a base pair) was first proposed by Leonard Lerman in 1961. One proposed mechanism of intercalation is as follows: In aqueous isotonic solution, the cationic intercalator is attracted electrostatically to the surface of the polyanionic DNA. The ligand displaces a sodium and/or magnesium cation present in the “condensation cloud” of such cations that surrounds DNA (to partially balance the sum of the negative charges carried by each phosphate oxygen), thus forming a weak electrostatic association with the outer surface of DNA. From this position, the ligand diffuses along the surface of the DNA and may slide into the hydrophobic environment found between two base pairs that may transiently “open” to form an intercalation site, allowing the ethidium to move away from the hydrophilic (aqueous) environment surrounding the DNA and into the intercalation site. The base pairs transiently form such openings due to energy absorbed during collisions with solvent molecules.

Some of the most commonly used intercalating DNA dyes include:

Ethidium Bromide. This intercalating agent experiences a roughly 20-fold increase in brightness on binding to DNA and is excellent for staining DNA in agarose gel electrophoresis. It fluoresces under UV light.

SYBR® Green. This highly sensitive DNA stain fluoresces under ultraviolet light. Helixyte™ Green has the same spectral properties to those of SYBR® Green. Helixyte™ Green has much greater sensitivity for dsDNA, thus especially useful for assays where the presence of contaminating RNA or ssDNA might obscure results.

SYBR® Safe. The major advantages of this DNA dye are that it is as sensitive as ethidium bromide and can be visualized without UV light. A blue light hox offers a safer option for visualization as the wavelengths do not cause DNA damage. SYBR® Safe was introduced as a safer alternative to EtBr and SYBR® Green, but unfortunately, it is much less sensitive than SYBR® Green. It only has sensitivity comparable to EtBr.

Gelite™ Safe. Gelite™ Safe has been developed specifically to be less hazardous than EtBr for staining DNA in agarose and acrylamide gels with much higher sensitivity. Gelite™ Safe has greatly improved safety and uncompromised sensitivity.

Propidium Iodide. This intercalating DNA dye exhibits a 20 to 30-fold increase in fluorescence on binding to DNA. The dye is membrane impermeable and can only enter cells with compromised membranes, making it an excellent probe for identifying dead cells.

SYBR® Gold. Upon binding to nucleic acids, this highly sensitive intercalating DNA dye exhibits 1000-fold greater UV fluorescence.

Crystal Violet. This is a highly sensitive dye that is detectable in the visible range, which eliminates the risks of UV exposure. It intercalates with DNA in a similar manner as ethidium bromide but is less mutagenic. It is an excellent option for detection of nucleic acid in gel electrophoresis.

DAPI (4′,6-diamidino-2-phenylindole). DAPI binds strongly to A-T rich regions of dsDNA. On binding it exhibits a roughly 20-fold increase in fluorescence. Its inability to pass through intact cell membranes easily makes DAPI more successful at staining dead cells or cells with compromised membranes rather than live cell staining.

7-AAD (7-aminoactinoinycin D). 7-AAD hinds strongly to the G-C rich regions of double-stranded DNA. Its large stokes shift makes it effective for use in multicolor analysis in conjunction with blue and green-fluorescent probes.

Gel Red. A robust, stable and sensitive DNA dye, Gel Red can be used as an in-gel stain or post stain. It is visualized using UV light.

LC Green/LC Green Plus. LC Green dyes are designed for high-resolution melting curve analysis to detect DNA sequence variants. The addition of these dyes tends to increase the melting temperature of DNA by 1-3° C., thus possibly requiring adjustment of cycling parameters. They are manufactured by Idaho Technology. They can detect heteroduplexes during melting analysis after PCR, are extremely stable, and do not inhibit PCR. LC Green PLUS is particularly useful in melting instruments with 96- or 384-well microtiter plates, has superb fluorescence intensity, and can be used with a variety of fluorescence-based PCR detection systems. LC Green I is a dsDNA binding dye used for Hi-Res Melting curve analysis and is particularly useful in HRM analysis to detect DNA sequence variants (SNPs, insertions/deletions).

### Applications

The potential applications for the technology disclosed herein is vast and virtually unlimited except by the need or desire to distinguish nucleic acid sequence variation using relatively low resource tools.

In one aspect, provided herein are methods of detecting sequence variation based on a comparison of melt temperature between an unknown double stranded D-DNA molecule and a reference double stranded D-DNA molecule with a first reference melt temperature and with a first reference elapsed melt time, the method comprising: (a) providing a double stranded L-DNA molecule with a second reference melt temperature and with a second reference elapsed melt time; (b) obtaining a first observed melt temperature and a first observed elapsed melt time for the double stranded L-DNA molecule under assay conditions; (c) obtaining a second observed melt temperature and a second observed elapsed melt time for the unknown double stranded D-DNA molecule under assay conditions identical to step (b); and (d) determining the difference between the first observed melt temperature provided by step (b) and the second observed melt temperature provided by step (c); wherein, when the difference of step (d) is not equal to the difference between the first reference melt temperature and the second reference melt temperature, then the unknown double stranded D-DNA molecule is identified as having a sequence variation relative to the reference double stranded D-DNA molecule.

In some embodiments, the method comprises detecting sequence variation based on a comparison of the elapsed melt time between the unknown double stranded D-DNA molecule and the reference double stranded D-DNA molecule with the first reference elapsed melt time, under assay conditions identical to step (b); and further comprising: (e) determining the difference between the elapsed melt times provided by step (b) and step (c); wherein, when the difference in step (e) is not equal to the difference between the first reference elapsed melt time and the second reference elapsed melt time, then the unknown double stranded D-DNA molecule is identified as having a sequence variation relative to the reference double stranded D-DNA molecule.

In some embodiments, the melt temperature and/or the elapsed melt time are obtained from a calibrated instrument. In some embodiments, the melt temperature and/or the elapsed melt time are obtained from a non-calibrated instrument.

In some embodiments, the reference double stranded D-DNA molecule that has a first known elapsed melt time, and wherein the double stranded L-DNA molecule has a second known elapsed melt time. In some embodiments, an elapsed melt time need not be obtained from a calibrated instrument. In some embodiments, the first known elapsed melt time and the second known elapsed melt time are the same. In some embodiments, the first known elapsed melt time and the second known elapsed melt time are different. In some embodiments, the first known elapsed melt time and the second known elapsed melt time are within about 5 seconds of each other. In some embodiments, the first known elapsed melt time and the second known elapsed melt time are different by a known amount. In some embodiments, the elapsed melt times are calculated from the second degree Savitsky-Golay polynomials at each point based on the first derivative of fluorescence with respect to elapsed melt time. In some embodiments, the elapsed melt time is derived from the raw data of melting ramp rate and fluorescence measurements during melting. In some embodiments, the elapsed melt time is defined as the time (in seconds) to reach the maximum derivative of fluorescence with respect to elapsed melt time.

In some embodiments, the melt temperature and/or the elapsed melt time of the unknown double stranded D-DNA molecule is determined using an intercalating dye, which may be selected from the group consisting of SYBR® Gold, SYBR® Green, EvaGreen, SYTO 82, SYTO 64, SYTO 9, and LCGreen dyes.

In some embodiments, the L-DNA is end-labeled with a dye on one strand and end-labeled with a quencher on the other strand. In some embodiments, the D-DNA is labeled with an intercalating dye that is not impacted by the quencher.

In some embodiments, the double stranded L-DNA is end-labeled on either the forward or reverse strand with a dye, and excitation is provided by a compatible intercalating dye.

In some embodiments, the concentration of the L-DNA is adjusted to provide a desired value for the second melt temperature or elapsed melt time. In some embodiments, the ratio of the forward: reverse strands of the L-DNA may be adjusted to provide a desired value for the second melt temperature or elapsed melt time. For example, the elapsed melt time for the L-DNA may be adjusted to approximate the elapsed melt time of the reference D-DNA using the ratio of the forward: reverse strands of L-DNA. The elapsed melt time for the L-DNA may be adjusted to such that it is a desired amount different from the elapsed melt time of the reference D-DNA using the ratio of the forward: reverse strands of L-DNA. The L-DNA total concentration and/or the L-DNA strand ratio may be such that the second known melt temperature or elapsed melt time of the L-DNA molecule is equal to the first known melt temperature or elapsed melt time of the reference double stranded D-DNA molecule. In some embodiments, the L-DNA concentration and/or strand ratio is adjusted according to the Van't Hoff equation. In some embodiments, the quencher-labeled strand of the L-DNA is present in a molar excess relative to the fluorophore labeled L-DNA strand. In some embodiments, the ratio of fluorophore labeled strand of the L-DNA to the quencher-labeled strand of the L-DNA is about 1:3. In some embodiments, about 1×10¹¹ copies of the fluorophore labeled L-DNA strand and about 3×10¹¹ copies of the quencher-labeled L-DNA strand are used.

In some embodiments, the unknown D-DNA is a template dependent amplification product, and the L-DNA is present in the template-dependent reaction mixture. In some embodiments, multiple distinct double stranded L-DNA sequences with multiple distinct melt temperatures or multiple distinct elapsed melt times are used together in the same reaction. In some embodiments, the unknown double stranded D-DNA molecule is provided using real time PCR performed in the presence of the double stranded L-DNA reference molecule in the same reaction. In some embodiments, steps (b) and (c) are performed simultaneously in the same reaction. In some embodiments, the sequence variation between the unknown double stranded D-DNA molecule and the reference double stranded D-DNA molecule is a single base change. In some embodiments, the L-DNA molecule and the reference D-DNA molecule do not have the same sequence. In some embodiments, multiple distinct L-DNA sequences with multiple melt properties are used together. In some embodiments, the method further comprises quantitating the test D-DNA products.

In some embodiments, the reference DNA has the same sequence as a natural D-DNA that is unique to a drug resistant pathogen. In some embodiments, the drug resistant pathogen comprises a drug resistant bacterium or drug resistant virus. In some embodiments, the drug-resistant pathogen is Mycobacterium tuberculosis, and the reference double stranded D-DNA sequence comprises GGCACCAGCCAGCTGAGCCAATTCATGGACCAGAACAACCCGCT GTCGGGGTTGACCCACAAGCGCCGACTGTCGGCGCTG (SEQ ID NO: 1), or a sequence at least about 70%, at least about 75%, at least about 80%, at least about 85%, at least about 90%, at least about 95%, at least about 96%, at least about 97%, at least about 98%, at least about 99%, or more, identical thereto.

In one aspect, provided herein is a method of detecting sequence variation between an unknown double stranded D-DNA molecule and a reference double stranded D-DNA molecule, the method comprising: (a) providing a melt probe having reverse complementarity to a drug-susceptible sequence in the unknown double stranded D-DNA molecule relative to a corresponding sequence in the reference double stranded D-DNA molecule; (b) providing a double stranded L-DNA molecule; (c) optionally adjusting the concentration of the melt probe and/or adjusting the concentration or the ratio of the forward: reverse strands of the double stranded L-DNA so that the melt temperatures or reference elapsed melt times are about identical between a melt probe: reference asymmetric PCR product duplex and the double stranded L-DNA molecule; (d) performing asymmetric PCR to provide one or more asymmetric PCR products of the unknown double stranded D-DNA molecule, wherein one or more melt probe: asymmetric PCR product duplexes are formed; (e) obtaining a first observed melt temperature and a first observed elapsed melt time for the double stranded L-DNA molecule under assay conditions; (f) obtaining a second observed melt temperature and a second elapsed melt time for the one or more melt probe: asymmetric PCR product duplexes under assay conditions identical to step (e); and (g) determining the difference between the first observed melt temperature or first elapsed melt time provided by step (e) and the second observed melt temperature or second elapsed melt time provided by step (f); wherein, if there is no difference in step (g), then the unknown double stranded D-DNA molecule is identical to the reference double stranded D-DNA molecule; and wherein, if there is a difference in step (g), then the unknown double stranded D-DNA molecule is identified as having a sequence variation relative to the reference double stranded D-DNA molecule.

In some embodiments, the forward strand of the double stranded L-DNA molecule is identical in sequence and in length to the melt probe. In some embodiments, the forward strand of the double stranded L-DNA molecule and the melt probe do not have the same sequence and/or length.

In some embodiments, the sequence variation detection is a drug susceptibility analysis. In some embodiments, the drug susceptibility analysis is performed for the drug rifampicin. In some embodiments, when the within-sample melt temperature difference, which is equal to the difference between the L-DNA melt temperature and the susceptible melt probe-reference asymmetric PCR product duplex, is greater than about 0.83° C., greater than about 0.84° C., greater than about 0.85° C., greater than about 0.9° C., or greater than about 1.0° C., then the sample is classified as rifampicin-resistant. In some embodiments, when the within-sample melt temperature difference is less than about 0.83° C., (otherwise referred to herein as the term “single susceptibility cutoff”) then the sample is classified as rifampicin-susceptible. In some embodiments, use of a single susceptibility cutoff to identify a sample as drug susceptible, or alternatively, drug resistant, eliminates instrument-specific optimization.

In some embodiments, the 5′ end of the susceptible melt probe is greater than 1 nucleotide, greater than 2 nucleotides, greater than 3 nucleotides, greater than 4 nucleotides, greater than 5 nucleotides, greater than 7 nucleotides, or more, distance from the nucleotide variation in the nucleotide variation in the drug susceptible gene. See, e.g., Table 14 of Example 2 herein.

In some embodiments, the melt temperatures or elapsed melt times are obtained using a calibrated instrument. In some embodiments, the melt temperatures or elapsed melt times are obtained using a non-calibrated instrument.

In some embodiments, the provided elapsed melt time is calculated from second degree Savitsky-Golay polynomials at each point based on the first derivative of fluorescence with respect to tm. In some embodiments, the elapsed melt time is derived from the raw data of melting ramp rate and fluorescence measurements during melting. In some embodiments, the elapsed melt time is defined as the time (sec) to reach maximum derivative of fluorescence with respect to tm.

In some embodiments, the sequence variation between the unknown double stranded D-DNA molecule and the reference double stranded D-DNA molecule is a single base change. In some embodiments, the double stranded L-DNA molecule and the reference D-DNA molecule do not have the same sequence.

In some embodiments, the L-DNA forward strand is synthesized with the same length and same sequence as the drug-susceptible melt probe. In some embodiments, the forward strand of the L-DNA molecule is end-labeled. In some embodiments, the reverse complement L-DNA strand is unlabeled.

In some embodiments, the asymmetric PCR is performed using a real-time PCR instrument as described herein. In some embodiments, the L-DNA melt data is fluorescence-based and is collected on a separate optical channel from the fluorescence-based D-DNA melt data. In some embodiments, the method comprises implementation of intercalated FRET (iFRET) to capture L-DNA melt data. In some embodiments, the susceptible melt probe: reference asymmetric PCR product duplex melt analysis is monitored on a green optical channel using an intercalating dye. In some embodiments, the susceptible melt probe: reference asymmetric PCR product duplex melt analysis is monitored on a green optical channel using a SYBR® green dye. In some embodiments, at least one strand of the double stranded L-DNA molecule is end-labeled with Texas Red. In some embodiments, the forward strand of the double stranded L-DNA molecule is end-labeled with Texas Red. In some embodiments, the L-DNA melting is monitored on the orange optical channel using iFRET, wherein SYBR® Green I is the FRET donor and Texas Red is the FRET acceptor. In some embodiments, the green optical channel comprises an excitation range between about 455 nanometers (nm) and about 485 nm and an emission range between about 505 nm and about 535 nm. In some embodiments, the orange optical channel comprises an excitation range between about 460 nanometers (nm) and about 480 nm and an emission range between about 605 nm and about 637 nm. In some embodiments, the orange optical channel comprises

In some embodiments, the melt analysis is provided by generating a positive melt curve derived from iFRET acceptor fluorescence decreases during melting, which is analyzed by the PCR instrumentation to calculate the melt temperature.

In some embodiments, the methods described herein can be multiplexed. In some embodiments, the methods described herein can screen two or more drug susceptibility regions in one or more drug susceptibility genes. In some embodiments, the method can screen at least two, at least three, at least four, at least five, or more, drug susceptibility regions in one or more drug susceptibility genes. In some embodiments, the method can screen at least two, at least three, at least four, at least five, or more, drug susceptibility regions in two or more drug susceptibility genes.

In some embodiments, the susceptible gene is rpoB and the susceptible melt probe mimic as reverse complement of wild-type rpoB comprises a sequence selected from SEQ ID NOs: 42, 45, 48, or 51, or a sequence at least about 70%, at least about 75%, at least about 80%, at least about 85%, at least about 90%, at least about 95%, at least about 96%, at least about 97%, at least about 98%, at least about 99%, or more, identical thereto.

In some embodiments, the susceptible gene is rpoB and the asymmetric PCR product mimic as wild-type rpoB comprises a sequence selected from SEQ ID NOs: 43, 46, 49, or 52, or a sequence at least about 70%, at least about 75%, at least about 80%, at least about 85%, at least about 90%, at least about 95%, at least about 96%, at least about 97%, at least about 98%, at least about 99%, or more, identical thereto.

In some embodiments, the susceptible gene is rpoB and the asymmetric PCR product mimic as rpoB variant comprises a sequence selected from SEQ ID NOs: 44, 47, 50, or 53, or a sequence at least about 70%, at least about 75%, at least about 80%, at least about 85%, at least about 90%, at least about 95%, at least about 96%, at least about 97%, at least about 98%, at least about 99%, or more, identical thereto.

In an alternative aspect, provided herein is a method of detecting sequence variation in an unknown double stranded D-DNA molecule as compared to a reference double stranded D-DNA molecule, the method comprising: (a) providing a control probe having reverse complementarity to a first sequence that is identical between the unknown double stranded D-DNA molecule and the reference double stranded D-DNA molecule; (b) providing a melt probe having reverse complementarity to a drug-susceptible sequence in the unknown double stranded D-DNA molecule relative to a corresponding sequence in the reference double stranded D-DNA molecule; (c) adjusting the concentration and/or ratios of the first probe and second probe so that the melt temperatures or elapsed melt times are identical between the control probe and the susceptible melt probe; (d) performing asymmetric PCR to provide one or more asymmetric PCR products; (e) obtaining a first melt temperature and a first elapsed melt time for a control probe: asymmetric PCR product duplex under assay conditions; (f) obtaining a second melt temperature and a second elapsed melt time for a susceptible melt probe: asymmetric PCR product duplex under assay conditions identical to step (e); and (g) determining the difference between the melt temperatures or elapsed melt times provided by step (e) and step (f); wherein, if there is no difference in step (g), then the unknown double stranded D-DNA molecule is identical to the reference double stranded D-DNA molecule; and wherein, if there is a difference step (g), then the unknown double stranded D-DNA molecule is identified as having a sequence variation relative to the reference double stranded D-DNA molecule.

In some embodiments, the method does not involve the use of L-DNA. In some embodiments, the method further comprises a double stranded L-DNA molecule for use as an internal melt standard.

In some embodiments, the asymmetric PCR is performed using a real-time PCR instrument as described herein.

In one aspect, the methods described herein are used in the assessment of variation the causative agents of infectious disease. There is significant interest in assessing disease causing viral or bacterial variants that have different disease course/risk and varying susceptibility to treatment. In some embodiments, the disease causing infection is selected from SARS-CoV-2, HIV, malaria, and tuberculosis infections.

In some embodiments, the methods described herein are used in assessing specific genetic information about an individual without incurring the expense of complete genome sequencing. This might include the identification of critical sequence information required for pharmaceutical intervention, such as testing for known genetic variants associated with known adverse reactions to a drug. This could translate into companion diagnostic to guide treatments towards or away from certain subjects and to determine which diseases (cancers, infections) should be treated with which drugs.

For a discussion of anti-microbial resistance and human gene variations, see Tong et al., 2012.

In some embodiments, the disease causing infection is a tuberculosis infection and the nucleic acid variation to be tested is a drug susceptible region of a gene in Mycobacterium tuberculosis. Mutations in Mycobacterium tuberculosis which are prominently associated with drug resistance are known to persons of skill in the art, for example as described in Catalogue of mutations in Mycobacterium tuberculosis complex and their association with drug resistance, second edition. Geneva: World Health Organization; 2023, incorporated herein by reference for all purposes, hereinafter referred to as “WHO 2023 T B Catalogue”.

In some embodiments, the drug susceptible gene is rpoB and the drug is rifampicin (RIF), or a derivative thereof. In some embodiments, the rifampicin derivative comprises rifapentine (RPT). In some embodiments, the drug susceptible region of rpoB comprises a codon selected from V170, D435, H445, S450, L452, L430, Q432, S441, F433, I491, or a combination thereof. In some embodiments, the rpoB variation comprises I491F, I491M, or I491N. In some embodiments, the rifampicin-susceptible mutation is a Group 1 or Group 2 resistance mutation as defined in the WHO 2023 T B Catalogue.

In some embodiments, the drug susceptible gene comprises rpoB and the forward primer comprises SEQ ID NO: 30, or a sequence at least about 70% identical thereto. In some embodiments, the drug susceptible gene comprises rpoB and the reverse primer comprises SEQ ID NO: 30, or a sequence at least about 70% identical thereto.

In some embodiments, the drug susceptible gene comprises rpoB and the drug-susceptible melt probe comprises SEQ ID NO: 32, or a sequence at least about 70% identical thereto. In some embodiments, the drug-susceptible melt probe further comprises a 3′ spacer. In some embodiments, the 3′ spacer comprises a 3′ C3 spacer (3SpC3).

In some embodiments, the drug susceptible gene comprises rpoB and the drug-susceptible L-DNA comparator forward strand comprises SEQ ID NO: 33, or a sequence at least about 70% identical thereto. In some embodiments, the L-DNA comparator is end-labeled. In some embodiments, the L-DNA comparator forward strand is end labeled with Texas Red. In some embodiments, the drug susceptible gene comprises rpoB and the drug-susceptible L-DNA comparator reverse complement strand comprises SEQ ID NO: 34, or a sequence at least about 70% identical thereto. In some embodiments, the L-DNA comparator reverse complement strand is not end labeled.

In some embodiments, the reference double-stranded D-DNA molecule comprises a sequence within SEQ ID NO: 38 of rpoB.

In some embodiments, the rpoB variation comprises a mutation selected from I491F, I491N, or I491M. In some embodiments, the rpoB variation comprises an alteration selected from 1471A>T, 1472T>A, 1473C>A, or a combination thereof. In some embodiments, the drug susceptible region of rpoB comprises a variation within SEQ ID NO: 39. In some embodiments, the drug susceptible region of rpoB comprises a variation within SEQ ID NO: 40. In some embodiments, the drug susceptible region of rpoB comprises a variation within SEQ ID NO: 41. In some embodiments, the drug susceptible gene is katG and the drug is isoniazid (INH), or a derivative thereof. In some embodiments, the drug susceptible region of katG comprises a codon selected from M1, S315, W328, or a combination thereof. In some embodiments, the katG variation is selected from MIX, S315R, S315N, S315I, S315T, or W328L. In some embodiments, the drug susceptible region comprises promoter mutations upstream of fabG1-inhA operon and the drug is isoniazid (INH), or a derivative thereof. In some embodiments, the variation comprises an alteration selected from −16A>G, −15C>T, −8T>A, or −8T>C, or a combination thereof, upstream of fabG1. In some embodiments, the variation comprises a fabG1 609G>A L203L mutation, which is known to create an alternative inhA promoter. In some embodiments, the variation comprises an inhA S94A mutation. In some embodiments, the isoniazid-susceptible mutation is a Group 1 or Group 2 resistance mutation as defined in the WHO 2023 T B Catalogue.

In some embodiments, the katG sequence has one or more nucleic acid base variations relative to SEQ ID NO: 2. In some embodiments, the variant katG sequence comprises a S315T mutation. In some embodiments, the variant katG sequence comprises a S315I mutation. In some embodiments, the variant katG sequence comprises a S315R mutation. In some embodiments, the variant katG sequence comprises a S315G mutation. In some embodiments, the variant katG sequence comprises a S315L mutation. In some embodiments, the variant katG sequence comprises an A312V mutation. In some embodiments, the variant katG sequence comprises a G316D mutation. In some embodiments, the variant katG sequence comprises a A312V. In some embodiments, the variant katG sequence comprises a S315T and A312V mutation. In some embodiments, the variant katG sequence comprises a S315T and G316D mutation. In some embodiments, the variant katG sequence comprises a S315T, G316D, and A312V mutation. In some embodiments, the variant katG sequence comprises SEQ ID NOs: 3-11, or a sequence at least about 70%, at least about 75%, at least about 80%, at least about 85%, at least about 90%, at least about 95%, at least about 96%, at least about 97%, at least about 98%, at least about 99%, or more, identical thereto.

In some embodiments, the katG sequence variation is selected from 944G>C, 944G>A, 944G>T, 945C>A, 943A>G, 943A>C, 935C>T, 947G>A, or a combination thereof.

In some embodiments the reference double-stranded D-DNA molecule comprises SEQ ID NO: 2. In some embodiments the unknown double-stranded D-DNA molecule comprises a sequence selected from SEQ ID NOs: 3-11, or a sequence at least about 70%, at least about 75%, at least about 80%, at least about 85%, at least about 90%, at least about 95%, at least about 96%, at least about 97%, at least about 98%, at least about 99%, or more, identical thereto.

In some embodiments, the reference double-stranded D-DNA molecule comprises a sequence within SEQ ID NO: 12 of katG.

In some embodiments, the reference double-stranded D-DNA molecule comprises a sequence within SEQ ID NO: 13 of katG. In some embodiments the unknown double-stranded D-DNA molecule comprises SEQ ID NO: 3, or a sequence at least about 70% identical thereto.

In some embodiments, the reference double-stranded D-DNA molecule comprises a sequence within SEQ ID NO: 14 of katG. In some embodiments the unknown double-stranded D-DNA molecule comprises SEQ ID NO: 4, or a sequence at least about 70% identical thereto.

In some embodiments, the reference double-stranded D-DNA molecule comprises a sequence within SEQ ID NO: 15 of katG. In some embodiments the unknown double-stranded D-DNA molecule comprises SEQ ID NO: 5, or a sequence at least about 70% identical thereto.

In some embodiments, the reference double-stranded D-DNA molecule comprises a sequence within SEQ ID NO: 16 of katG. In some embodiments the unknown double-stranded D-DNA molecule comprises SEQ ID NO: 6, or a sequence at least about 70% identical thereto.

In some embodiments, the reference double-stranded D-DNA molecule comprises a sequence within SEQ ID NO: 17 of katG. In some embodiments the unknown double-stranded D-DNA molecule comprises SEQ ID NO: 7, or a sequence at least about 70% identical thereto.

In some embodiments, the reference double-stranded D-DNA molecule comprises a sequence within SEQ ID NO: 18 of katG. In some embodiments the unknown double-stranded D-DNA molecule comprises SEQ ID NO: 8, or a sequence at least about 70% identical thereto.

In some embodiments, the reference double-stranded D-DNA molecule comprises a sequence within SEQ ID NO: 19 of katG. In some embodiments the unknown double-stranded D-DNA molecule comprises SEQ ID NO: 9, or a sequence at least about 70% identical thereto.

In some embodiments, the reference double-stranded D-DNA molecule comprises a sequence within SEQ ID NO: 20 of katG. In some embodiments the unknown double-stranded D-DNA molecule comprises SEQ ID NO: 10, or a sequence at least about 70% identical thereto.

In some embodiments, the reference double-stranded D-DNA molecule comprises a sequence within SEQ ID NO: 21 of katG. In some embodiments the unknown double-stranded D-DNA molecule comprises SEQ ID NO: 11, or a sequence at least about 70% identical thereto.

In one aspect, provided herein is an L-DNA sequence that is end-labeled. In some embodiments, the L-DNA comprises SEQ ID NO: 22, or a sequence at least about 70% identical thereto, and the end-label comprises Texas Red. In some embodiments, the L-DNA comprises SEQ ID NO: 23, or a sequence at least about 70% identical thereto, and the end-label comprises BHQ2. In some embodiments, the L-DNA comprises SEQ ID NO: 28, or a sequence at least about 70% identical thereto. In some embodiments, the L-DNA comprises SEQ ID NO: 29, or a sequence at least about 70% identical thereto. In some embodiments, the end-labeling occurs on the 5′ terminus of the L-DNA sequence. In some embodiments, the end-labeling occurs on the 3′ terminus of the L-DNA sequence.

In some embodiments, the wild-type drug-susceptible amplicon forward strand comprises SEQ ID NO: 26, or a sequence at least about 70% identical thereto. In some embodiments, the wild-type drug-susceptible amplicon reverse strand comprises SEQ ID NO: 27, or a sequence at least about 70% identical thereto.

In some embodiments, the drug susceptible gene is embB or embA and the drug is ethambutol (EMB), or a derivative thereof. In some embodiments, the drug susceptible region of embB comprises a codon selected from L74, M306, Q497, G406, D354, Y319, D328, or a combination thereof. In some embodiments, the embB variation is selected from M306V, M306I, Q497R, G406A, D354A, G406D, Y319S, G406S, Q497L, M306L, D328Y, G406C, or Y319C. In some embodiments, the ethambutol-susceptible mutation is a Group 1 or Group 2 resistance mutation as defined in the WHO 2023 T B Catalogue.

In some embodiments, the drug susceptible gene is pncA and the drug is pyrazinamide (PZA), or a derivative thereof. In some embodiments, the drug susceptible region of pncA comprises a codon selected from H57, H51, Q141, Q10, V139, G132, V131, T76, G97, L27, D49, T135, C14, L4, H71, E173, T153, V7, P54, W68, L172, I133, L120, I55, P69, T142, V128, A134, D12, G97, F58, D129, or a combination thereof. In some embodiments, the pncA variation comprises a pncA loss of function mutation. In some embodiments, the pyrazinamide-susceptible mutation is a Group 1 or Group 2 resistance mutation as defined in the WHO 2023 T B Catalogue.

In some embodiments, the drug susceptible gene is gyrA or gyrB and the drug is levofloxin (LFX) or moxifloxacin, or a derivative thereof. In some embodiments, the drug susceptible region of gyrA comprises a codon selected from D94, A90, S91, G88, D89, or a combination thereof. In some embodiments, the drug susceptible region of gyrB comprises a codon selected from N499, E501, A504, S447, N499, or a combination thereof. In some embodiments, the levofloxin- or moxifloxacin-susceptible mutation is a Group 1 or Group 2 resistance mutation as defined in the WHO 2023 T B Catalogue.

In some embodiments, the drug susceptible gene is Rv0678, atpE, or pepQ, and the drug is bedaquiline (BEQ) or clofazimine (CFZ), or a derivative thereof. In some embodiments, the drug susceptible region of Rv0678 comprises a codon selected from E49, D47, I67, G121, L117, M146, C46, A63, A36, N70, L32, or a combination thereof. In some embodiments, the drug susceptible region of atpE comprises a codon selected from A63, I66, or a combination thereof. In some embodiments, the drug susceptible region of Rv0678 comprises a loss of function mutation. In some embodiments, the drug susceptible region of pepQ comprises a loss of function mutation. In some embodiments, the bedaquiline- or clofazimine-susceptible mutation is a Group 1 or Group 2 resistance mutation as defined in the WHO 2023 T B Catalogue.

In some embodiments, the drug susceptible gene is rplC and the drug is linezolid (LZD), or a derivative thereof. In some embodiments, the drug susceptible region of rplC comprises a C154 codon. In some embodiments, the drug susceptible region of rplC comprises a C154R mutation. In some embodiments, the drug susceptible region of rplC comprises a 2814G>T alteration. In some embodiments, the linezolid-susceptible mutation is a Group 1 or Group 2 resistance mutation as defined in the WHO 2023 T B Catalogue.

In some embodiments, the drug susceptible gene is ddn and the drug is delamanid (DLM) or pretomanid, or a derivative thereof. In some embodiments, the drug susceptible region of ddn comprises a L49 codon. In some embodiments, the drug susceptible region of ddn comprises a L49P mutation. In some embodiments, the drug susceptible region of ddn comprises a loss of function mutation. In some embodiments, the delamanid- or pretomanid-susceptible mutation is a Group 1 or Group 2 resistance mutation as defined in the WHO 2023 T B Catalogue.

In some embodiments, the drug susceptible gene is eis and the drug is amikacin (AMK), or a derivative thereof. In some embodiments, the drug susceptible region of eis comprises a loss of function mutation. In some embodiments, the drug susceptible region of rplC comprises a 1401A>G or 1484G>T alteration. In some embodiments, the amikacin-susceptible mutation is a Group 1 or Group 2 resistance mutation as defined in the WHO 2023 T B Catalogue.

In some embodiments, the drug susceptible gene is rpsL, rrs, or gid and the drug is streptomycin (STM), or a derivative thereof. In some embodiments, the drug susceptible region of rpsL comprises a codon selected from L43, L88, L88, or a combination thereof. In some embodiments, the drug susceptible region of gid comprises a codon selected from R39, N125, E99, A134, V105, H48, S70, G73, A200, P84, or a combination thereof. In some embodiments, the drug susceptible region of rrs comprises a 517C>T or 514A>C alteration. In some embodiments, the streptomycin-susceptible mutation is a Group 1 or Group 2 resistance mutation as defined in the WHO 2023 T B Catalogue.

In some embodiments, the drug susceptible gene is inhA or ethA and the drug is ethionamide (ETO) or prothionamide, or a derivative thereof. In some embodiments, the drug susceptible region of ethA comprises a codon selected from L37, M1, R207, Y235, N379, S390, A341, L35, S57, T88, V202, C403, or a combination thereof. In some embodiments, the drug susceptible region of inhA comprises an alteration selected from 777C>T, 154G>A, 770T>C, 779G>T, 770T>C, 770T>G, or a combination thereof. In some embodiments, the ethionamide- or prothionamide-susceptible mutation is a Group 1 or Group 2 resistance mutation as defined in the WHO 2023 T B Catalogue.

In alternative embodiments, the methods described herein are adapted to other mutation hotspots including but not limited to cancer-associated mutations, other drug susceptibility regions, or gene mutations associated with drug side effects. Once an associations between a nucleic acid variation and a certain susceptible phenotype is established, the methods described herein can be implemented.

In some embodiments, the method comprises: (i) designing PCR primers to amplify the target screening region; (ii) selecting a susceptible melt probe to define a nucleotide screening span; (iii) optimizing the susceptible melt probe to maximize mutation-induced melt shifts; (iv) providing a double stranded L-DNA molecule by synthesis to mimic the susceptible duplex formed by the melt probe hybridized to a wild-type asymmetric PCR product; (v) optionally adjusting the melt probe concentration to align the melt temperatures of the probe-wild-type product duplex with the susceptible L-DNA. Advantageously, the melt temperature difference remains constant for a fixed probe concentration, ensuring reliable sample classification even without matching the melt curves.

In other embodiments, a specific example of “companion” diagnostic application would be in conjunction with receptor tyrosine kinase genes in non-small cell lung cancer (NSCLC) patients (Paez et al., 2004) described somatic mutations of the epidermal growth factor receptor gene EGFR found in 15 of 58 unselected tumors from Japan and 1 of 61 from the United States. Treatment with the EGFR kinase inhibitor gefitinib (lressa) causes tumor regression in some patients with NSCLC, more frequently in Japan. EGFR mutations were found in additional lung cancer samples from U.S. patients who responded to gefitinib therapy and in a lung adenocarcinoma cell line that was hypersensitive to growth inhibition by gefitinib, but not in gefitinib-insensitive tumors or cell lines. These results suggest that EGFR mutations may predict sensitivity to gefitinib.

A specific example of genetic screening for disease is sickle cell screening (Yue et al., 2014) compared complex sequencing methods to HRM methods for screening of sickle cell disease. Their study examined 511 individuals on the island of Bioko (Equatorial Guinea) and attempted to establish a method for rapid sickle cell disease screening. Following DNA extraction and polymerase chain reaction (PCR) amplification, high resolution melting (HRM) analysis was used to assess the specificity of fluorescence signals of the PCR products and to differentiate various genotypes of these products. The analytical results of HRM were validated using DNA sequencing. By HRM analysis, 80 out of 511 samples were classified as hemoglobin S (Hb S) heterozygotes, while 431 out of 511 samples were classified as wild-type. No mutant homozygote was identified. DNA sequencing indicated that within the 431 wild-type samples as indicated by HRM analysis, one case was actually a Hb S heterozygote and another case was a rare hemoglobin S-C genotype (sickle-hemoglobin C disease). One out of 80 suspected Hb S heterozygotes as indicated by HRM was confirmed as wild-type by DNA sequencing and the results of residual 508 cases were consistent for HRM analysis and sequencing. This suggests that HRM analysis is a simple, high-efficiency approach for Hb S screening and is useful for early diagnosis of SCD and particularly suitable for application in the African area.

In alternative aspects, the methods described herein are used to assess presequence screening, single nucleotide polymorphism (SNP) typing, methylation analysis, quantification (copy number variants and mosaicism), or as an alternative to gel-electrophoresis and clone characterization. See Vossen et al., 2009.

### Kits

In still further aspects, the present disclosure concerns kits for use with the methods described herein. In some embodiments, the L-DNA will be included in the kit. In some embodiments, the kits comprise, in suitable container means, an L-DNA, and optionally reagents including dyes, labels, standards, detection devices, and instructions for using the same.

In some embodiments, the L-DNA and other reagents may further comprise suitably aliquoted compositions. In some embodiments, the kits comprise individual reagents or mixed reagents, in one or more separate containers. In some embodiments, the components of the kits are packaged either in aqueous media or in lyophilized form.

In some embodiments, the containers of the kits include at least one vial, test tube, flask, bottle, syringe or other container means, into which the L-DNA may be placed, or preferably, suitably aliquoted. In some embodiments, the kits of the present disclosure will also typically include a means for containing the reagent containers in close confinement for commercial sale. In some embodiments, the containers include injection or blow-molded plastic containers into which the desired vials are retained.

In some embodiments, L-DNA is not included in the kit.

### Embodiments

1. A method of detecting sequence variation in a test double stranded D-DNA molecule as compared to a reference double stranded D-DNA molecule that has a first known melt property, the method comprising:
(a) providing a double stranded L-DNA molecule that has a second known melt property;
(b) obtaining a melt analysis providing a melt property for the double stranded L-DNA molecule;
(c) obtaining a melt analysis providing a melt property for the test double stranded D-DNA molecule under conditions identical to step (b); and
(d) determining the difference between the melt properties of the melt analyses of step (b) and step (c),
   Wherein when the difference between the melt properties of the melt analyses of step (b) and step (c) is not equal to the difference between the first known melt property and the second known melt property, then the test double stranded D-DNA molecule is identified as having a sequence variation relative to the reference double stranded D-DNA molecule.
   2. The method of embodiment 1, wherein the first known melt property and the second known melt property are each a melt temperature (e.g., from a calibrated instrument).
   3. The method of embodiment 1, wherein the melt analysis in (b) and (c) provide a melt temperature.
   4. The method of embodiment 1, wherein the first known melt property and the second known melt property are each an elapsed melt time.
   5. The method of embodiment 1, wherein the melt analysis in (b) and (c) provide an elapsed melt time (tm) that is calculated from the second degree Savitsky-Golay polynomials at each point based on the first derivative of fluorescence with respect to elapsed melt time.
   6. The method of embodiment 4 or 5, wherein the elapsed melt time is derived from the raw data of melting ramp rate and fluorescence measurements during melting.
   7. The method of any one of embodiments 4-6, wherein the elapsed melt time is defined as the time (in seconds) to reach the maximum derivative of fluorescence with respect to elapsed melt time.
   8. The method of any one of embodiments 1-7, wherein the first known elapsed melt time and the second known elapsed melt time are the same.
   9. The method of any one of embodiments 1-7, wherein the first known elapsed melt time and the second known elapsed melt time are within about 5 seconds of each other.
   11. The method of any one of embodiments 1-10, wherein said test double stranded D-DNA molecule is provided using real time PCR performed in the presence of the double stranded L-DNA molecule.
   12. The method of any one of embodiments 1-11, wherein the sequence variation between the test double stranded D-DNA molecule and the reference double stranded D-DNA molecule is a single base change.
   13. The method of any one of embodiments 1-12, wherein the L-DNA molecule and the reference D-DNA molecule do not have the same sequence.
   14. The method of any one of embodiments 1-13, wherein the melt temperature of the test double stranded D-DNA molecule is determined using an intercalating dye.
   15. The method of embodiment 14, wherein the intercalating dye is selected from the group consisting of SYBR® Gold, SYBR® Green, EvaGreen, SYTO 82, SYTO 64, SYTO 9, and LCGreen dyes.
   16. The method of any one of embodiments 1-15, wherein the L-DNA is end-labeled one strand with a dye and end-labeled with quencher on the other strand, and the D-DNA is labeled with an intercalating dye that is not impacted by the quencher.
   17. The method of any one of embodiments 1-16, wherein the concentration of the L-DNA is adjusted to provide a desired melt property.
   18. The method of any one of embodiments 1-17, wherein the ratio of the forward: reverse strands of L-DNA is adjusted to provide a desired melt property.
   19. The method of any one of embodiments 1-18, wherein the elapsed melt time for the L-DNA is adjusted to approximate the elapsed melt time of the reference D-DNA using the ratio of the forward: reverse strands of L-DNA.
   20. The method of any one of embodiments 1-18, wherein the elapsed melt time for the L-DNA is adjusted to such that it is a desired amount different from the elapsed melt time of the reference D-DNA using the ratio of the forward: reverse strands of L-DNA.
   21. The method of any one of embodiments 8-20, wherein the L-DNA total concentration and/or the L-DNA strand ratio are such that the second known melt property of the L-DNA molecule is equal to the first known melt property of the reference double stranded D-DNA molecule.
   22. The method of embodiment 20 or 21, wherein the L-DNA concentration and/or strand ratio are adjusted according to the Van't Hoff equation.
   23. The method of any one of embodiments 20-22, wherein the quencher labeled strand of the L-DNA is present in a molar excess relative to the fluorophore labeled L-DNA strand.
   24. The method of embodiment 23, wherein the ratio of fluorophore labeled strand of the L-DNA to the quencher labeled strand of the L-DNA is present is about 1:3.
   25. The method of embodiment 24, wherein 1×10¹¹ copies of the fluorophore labeled L-DNA strand and 3×10¹¹ copies of the quencher labeled L-DNA strand are used.
   26. The method of any one of embodiments 1-25, wherein steps (b) and (c) are performed simultaneously in the same reaction mixture.
   27. The method of any one of embodiments 1-26, wherein said test D-DNA is a template dependent amplification product.
   28. The method of any one of embodiments 1-27, wherein multiple distinct L-DNA sequences with multiple melt properties are used together.
   29. The method of any one of embodiments 1-28, wherein the D-DNA is a template-dependent amplification product, such as wherein the L-DNA is present in template-dependent reaction mixture.
   30. The method of any one of embodiments 1-29, further comprising quantitating D-DNA products.
   31. The method of any one of embodiments 1-30, wherein the reference DNA is one that has the same sequence as a natural D-DNA that is unique to a drug resistant pathogen, such as a drug resistant bacterium or virus.
   32. The method of embodiment 31, wherein the pathogen is Mycobacterium tuberculosis, and the sequence is GGCACCAGCCAGCTGAGCCAATTCATGGACCAGAACAACCCGCT GTCGGGGTTGACCCACAAGCGCCGACTGTCGG CGCTG (SEQ ID NO: 1).
   33. A kit comprising a double stranded L-DNA and at least one detectable moiety.
   34. The kit of embodiment 33, wherein the L-DNA has the same sequence as a naturally occurring D-DNA sequence, or wherein the L-DNA has less than 10% base differences as compared to the naturally occurring D-DNA sequence.
   35. The kit of embodiment 33 or 34, wherein the L-DNA comprises partial sequence pairs for molecular beacon detection.
   36. The kit of any one of embodiments 33-35, further comprising a melt detection reagent.
   37. The kit of embodiment 36, wherein the melt detection reagent is an intercalating dye that intercalate within both L-DNA and D-DNA, an intercalating dye that preferentially intercalates within L-DNA or D-DNA, a fluorescence-quencher pair, or an end-labelling reagent, such as wherein the end-labelling reagent is disposed on the 3′ and 5′ ends of two L-DNA complementary strands whose interaction is detectable.
   38. The kit of embodiment 36, wherein the melt detection reagent is one or more modified L-DNA bases incorporated into said L-DNA that exhibit electrical sensing changes during melting.
   39. The kit of any one of embodiments 33-38, further comprising one or more additives that modify melting differences, such as salts or betaine.
   40. The kit of any one of embodiments 33-39, further comprising:
(a) a device to perform heating of mixture over a range of room temperature to 99° C.;
(b) a device to measure fluorescence;
(c) a device to measure absorbance of light (e.g., a UV light emitting diode);
(d) a device to measure electrical changes of solution;
(e) a microprocessor (e.g., raspberry PI); and/or
(f) a “smart” device with sensing capabilities.
   41. The kit of any one of embodiments 33-40, wherein the kit further comprises instructions for using the kit and its components, such as in a method according to the present disclosure.

### EXAMPLES

The following examples are included to demonstrate preferred embodiments. It should be appreciated by those of skill in the art that the techniques disclosed in the examples that follow represent techniques discovered by the inventor to function well in the practice of embodiments and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1. L-DNA-Based Melt Analysis Enables within-Sample Validation of PCR Products

The melt analysis feature in most real-time polymerase chain reaction (PCR) instruments is a simple method for determining if expected or unexpected products are present. High resolution melt analysis (HRM) seeks to improve the precision of melt temperature measurements for better PCR product sequence characterization. In the area of tuberculosis (TB) drug susceptibility screening, sequencing has shown that a single base change can be sufficient to make a first-line TB drug ineffective. In this study, a reagent-based calibration strategy based on synthetic left-handed (L)-DNA, designated LHRM, was developed to confirm validation of a PCR product with single base resolution. To test this approach, a constant amount of a double-stranded L-DNA melt comparator was added to each sample and used as a within-sample melt standard. The performance of LHRM and standard HRM were used to classify PCR products as drug-susceptible or not drug-susceptible with a test bed of nine synthetic katG variants, each containing single or multiple base mutations that are known to confer resistance to the first-line TB drug isoniazid (INH). LHRM achieved comparable classification to standard HRM relying only on within-sample melt differences between L-DNA and the unknown PCR product. Using a state-of-the-art calibrated instrument and multiple sample classification analysis, standard HRM performed at 66.7% sensitivity and 98.8% specificity. Single sample analysis incorporating L-DNA for reagent-based calibration into every sample maintained high performance at 77.8% sensitivity and 98.7% specificity. LHRM shows promise as a high-resolution single sample method for validating PCR products in applications where the expected sequence is known.

### INTRODUCTION

Sequencing of tuberculosis (TB) drug-resistant strains has shown that many drug-resistant variants have one or more single nucleotide polymorphisms (SNPs) often clustered in contiguous regions of the drug-resistant genome. These resistance-related characteristic changes provide potential biomarkers for drug treatment decisions. However, requiring sequencing for every positive TB sample as part of a clinical treatment algorithm remains cost-prohibitive in many resource-constrained settings.

When the presence of drug-susceptible cases is high, a follow-on test to confirm drug susceptibility provides a pragmatic first step in a clinical treatment algorithm. The goal is to confirm drug susceptibility for the majority of samples and focus the limited resources on more complex testing for the small number of cases that are not drug-susceptible.

Based on known TB sequencing data, amplification-based susceptibility testing has been used to characterize samples and inform the drug treatment algorithm. These approaches are divided into two categories, direct or indirect testing. Direct testing confirms the presence of one or more specific SNPs that make the strain untreatable by a particular drug. Alternatively, indirect testing, based on the SNP clustering observation, broadly seeks to confirm the presence of the non-mutated, susceptible sequence that makes the strain treatable by a particular drug.

In the presence of multiple SNPs that independently confer resistance, the former SNP-targeted approach requires either implementation of a separate test for each SNP or a multiplexed design including all SNPs. Although this direct testing approach shows promise for particular TB strains of concern, it remains difficult to scale as the number of drug-resistant SNPs increases.

The latter cluster-based strategy for susceptibility testing is based on polymerase chain reaction (PCR) detection. This approach seeks to validate that the PCR product has the known drug-susceptible sequence by melt analysis. Melt analysis is based on the observation that any given double-stranded DNA sequence dissociates at a characteristic melt temperature (Tm). This property is used to compare the melt temperature of an unknown PCR product to the characteristic melt temperature of the known drug-susceptible wild-type sequence. Any shift from this wild-type melt temperature this implies that the unknown test sample contains one or more SNPs. Melt analysis capitalizes on the hardware capabilities of PCR instrumentation and is often available in real-time PCR instruments. Some real-time instruments also offer high resolution melt (HRM) capabilities by including a temperature calibration feature. Because of the many variables that affect the melt properties, standard HRM classifies an unknown sample by comparing the melt temperature of the unknown PCR product to the melt temperature of a known PCR product, usually included in as additional samples in the assay. The requirement for instrument calibration to enable the comparison of two or more samples is a major source of complexity in these approaches. In the case of current PCR and melt-based TB drug susceptibility tests Xpert MTB/RIF Ultra11 and Xpert MTB/XDR12, unknown samples are compared to an algorithm-based reference library of Tm signatures from a set of known mutations. Proprietary designs around this relatively expensive technology, however, continue to limit its utility, particularly where it is most needed. Since a limited number of point-of-care diagnostics currently offer low-cost TB drug resistance testing by HRM, there is a demonstrated need for simpler validation of drug susceptibility in the TB treatment algorithm using more widely-available real-time PCR instruments.

In this report, a potential single sample approach based on reagent-based calibration is proposed to simplify HRM and avoid the requirement for multiple sample comparisons in every assay. In this design, left-helical (L)-DNA is added to every sample as a standard melt comparator. The approach is based on the assumption that both double-stranded L-DNA additive and double-stranded D-DNA PCR product in the same well are affected by hybridization melt characteristics in the same way. If the melt characteristics of the L-DNA additive and the D-DNA from the PCR amplicon of a drug susceptible sample are set identical, any difference in melt characteristics between L-DNA and an unknown PCR product is attributed to a change in PCR product sequence. In other words, the drug-susceptible reference sequence is included for comparison to the sample PCR amplicon, not in a separate well as D-DNA, but rather within each sample as L-DNA.

Two key features of L-DNA support the feasibility of this approach. First, published reports suggest that L-DNA does not interfere or participate in PCR reactions and has been employed in applications where it does not interact with normal biological processes, such as intracellular biosensing and PCR control. Second, several reports suggest that L-DNA and naturally occurring (D)-DNA with identical sequences have identical melt characteristics, suggesting that matching melt characteristics should be possible.

Performance of L-DNA-based HRM (LHRM) was compared to standard HRM using a state-of-the-art HRM instrument for both methods. The assays were applied to drug susceptibility screening for isoniazid (INH), a first-line prodrug therapeutic for TB. The internal comparator L-DNA was synthesized as a 56-nucelotide sequence from the drug-susceptible TB katG gene where over 250 INH-resistance-related mutations are clustered. The nine synthetic variants were selected to provide product validation challenges that ranged from relatively easy, due to multi-base mutations, to very difficult, due to only a single base mutation. PCR products of these synthetic targets were classified as drug-susceptible or not drug-susceptible and used to compare the LHRM and standard HRM methods.

### DNA Oligonucleotide Design

The melt analysis test bed was developed using the drug-susceptible TB katG gene. A single primer set was designed to cover the most prevalent variant S315T found in 94% of INH-resistant clinical isolates and a subset of the many single or multi-base variants in the neighboring region that also confer INH resistance. Single-stranded PCR targets were synthesized with D-DNA sequences of drug-susceptible wild-type katG (H37Rv: 2153889-2156111) and nine clinically relevant drug-resistant katG mutants (Table 1).

TABLE 1
Drug-susceptible wild-type katG and nine katG variant sequences

| | | | Theoretical |
|---|---|---|---|
| Identifier | Sequence | Variant | Tm Diff (° C.) |
| SEQ ID NO: 2 | GCGATCACCAGCGGC | Wild-type | 0 |
| SEQ ID NO: 3 | GCGATCACCACCGGC | S315T | −0.68 |
| SEQ ID NO: 4 | GCGATCACCAACGGC | S315N | −1.03 |
| SEQ ID NO: 5 | GCGATCACCATCGGC | S315I | −0.87 |
| SEQ ID NO: 6 | GCGATCACCAGAGGC | S315R | −0.84 |
| SEQ ID NO: 7 | GCGATCACCGGCGGC | S315G | 0.33 |
| SEQ ID NO: 8 | GCGATCACCCTCGGC | S315L | −0.47 |
| SEQ ID NO: 9 | GTGATCACCACCGGC | S315T + A312V | −1.70 |
| SEQ ID NO: 10 | GCGATCACCACCGAC | S315T + G316D | −1.44 |
| SEQ ID NO: 11 | GTGATCACCACCGAC | S315T + G316D + | −2.47 |
| | | A312V | |

In Table 1, sequences (illustrated 5′-to-3′) of the drug-susceptible wild-type katG (SEQ ID NO: 2) and nine katG variants (SEQ ID NOs: 3-11) are shown. Each variant has INH-resistance-related mutations (underlined) that induce theoretical melt differences (Tm diff.) from wild-type (right column).

The selected variants included a range of melt differences from wild-type, offering both easy and challenging drug susceptibility classification cases. The theoretical Tm spread of variants was 2.8° C. based on a nearest neighbor oligonucleotide calculator with automated settings. Detailed information on the DNA oligonucleotide sequences used in these studies is shown in Table 2 below.

TABLE 2
Oligonucleotide sequences designed for standard HRM and LHRM

| | | | | NA | |
|---|---|---|---|---|---|
| | | | AA | Base | |
| ID | DNA Type | Description | Change | Change | Sequence (5′>3′) |
| MEP183 | D-DNA | Wild-type drug- | None | None | (SEQ ID NO: 12) |
| | | susceptible target | | | ATCTGGTCGGCCCCGAACCCGAG |
| | | | | | GCTGCTCCGCTGGAGCAGATGGG |
| | | | | | CTTGGGCTGGAAGAGCTCGTATG |
| | | | | | GCACCGGAACCGGTAAGGACGC |
| | | | | | GATCACCAGCGGCATCGAGGTCG |
| | | | | | TATGGACGAACACCCCGACGAAA |
| | | | | | TGGGACAACAGTTTCCTCGAGAT |
| | | | | | CCTGTACGGCTACGAGTGGGAGC |
| | | | | | TGACGAAGAGCCCTGCT |
| MEP184 | D-DNA | Mutant target | S315T | G944C | (SEQ ID NO: 13) |
| | | | | | CTCGTATGGCACCGGAACCGGTA |
| | | | | | 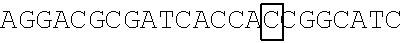 |
| | | | | | GAGGTCGTATGGACGAACACCCC |
| | | | | | GACGAAATGGGACAACAGTT |
| MEP185 | D-DNA | Mutant target | S315N | G944A | (SEQ ID NO: 14) |
| | | | | | CTCGTATGGCACCGGAACCGGTA |
| | | | | | 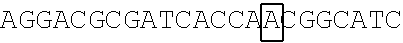 |
| | | | | | GAGGTCGTATGGACGAACACCCC |
| | | | | | GACGAAATGGGACAACAGTT |
| MEP186 | D-DNA | Mutant target | S315I | G944T | (SEQ ID NO: 15) |
| | | | | | CTCGTATGGCACCGGAACCGGTA |
| | | | | | 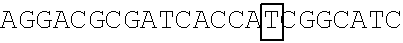 |
| | | | | | GAGGTCGTATGGACGAACACCCC |
| | | | | | GACGAAATGGGACAACAGTT |
| MEP187 | D-DNA | Mutant target | S315R | C945A | (SEQ ID NO: 16) |
| | | | | | CTCGTATGGCACCGGAACCGGTA |
| | | | | | 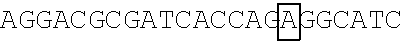 |
| | | | | | GAGGTCGTATGGACGAACACCCC |
| | | | | | GACGAAATGGGACAACAGTT |
| MEP188 | D-DNA | Mutant target | S315G | A943G | (SEQ ID NO: 17) |
| | | | | | CTCGTATGGCACCGGAACCGGTA |
| | | | | | 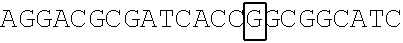 |
| | | | | | GAGGTCGTATGGACGAACACCCC |
| | | | | | GACGAAATGGGACAACAGTT |
| MEP189 | D-DNA | Mutant target | S315L | A943C + | (SEQ ID NO: 18) |
| | | | | G944T | CTCGTATGGCACCGGAACCGGTA |
| | | | | | AGGACGCGATCACCCTCGGCATC |
| | | | | | GAGGTCGTATGGACGAACACCCC |
| | | | | | GACGAAATGGGACAACAGTT |
| MEP197 | D-DNA | Mutant target | S315T + | G944C + | (SEQ ID NO: 19) |
| | | | A312V | C935T | 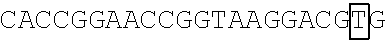 |
| | | | | | 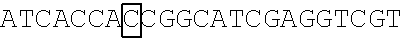 |
| | | | | | ATGGACGAACA |
| MEP198 | D-DNA | Mutant target | S315T + | G944C + | (SEQ ID NO: 20) |
| | | | G316D | G947A | CACCGGAACCGGTAAGGACGCG |
| | | | | | 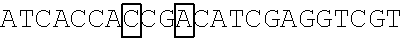 |
| | | | | | ATGGACGAACA |
| MEP199 | D-DNA | Mutant target | S315T + | G944C + | (SEQ ID NO: 21) |
| | | | G316D + | G947A + | 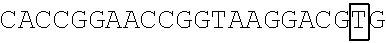 |
| | | | A312V | C935T | 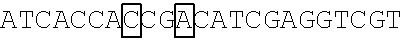 |
| | | | | | ATGGACGAACA |
| 23FEB_ka | L-DNA | Wild-type drug- | None | None | (SEQ ID NO: 22) |
| tGf56_ | | susceptible | | | /TXR/ |
| TXR | | comparator forward | | | CACCGGAACCGGTAAGGACGCG |
| | | strand with end- | | | ATCACCAGCGGCATCGAGGTCGT |
| | | labeling | | | ATGGACGAACA |
| 23FEB_ka | L-DNA | Wild-type drug- | None | None | (SEQ ID NO: 23) |
| tGf56_Rc | | susceptible | | | TGTTCGTCCATACGACCTCGATGC |
| mp + 5_BH | | comparator reverse | | | CGCTGGTGATCGCGTCCTTACCG |
| Q2 | | strand with end- | | | GTTCCGGTGCCATA/BHQ2/ |
| | | labeling | | | |
| MEP176 | D-DNA | 56 bp PCR amplicon | N/A | N/A | (SEQ ID NO: 24) |
| | | forward primer | | | CACCGGAACCGGTAAGG |
| MEP177 | D-DNA | 56 bp PCR amplicon | N/A | N/A | (SEQ ID NO: 25) |
| | | reverse primer | | | TGTTCGTCCATACGACCTC |

DNA is denoted as D-DNA or L-DNA;

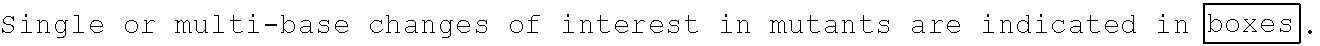

All DNA oligonucleotides employed for development and testing of the assay were synthesized by Integrated DNA technologies (Coralville, Iowa, USA) or biomers (Ulm, Baden-Württemberg, Germany).

### Standard HRM Approach

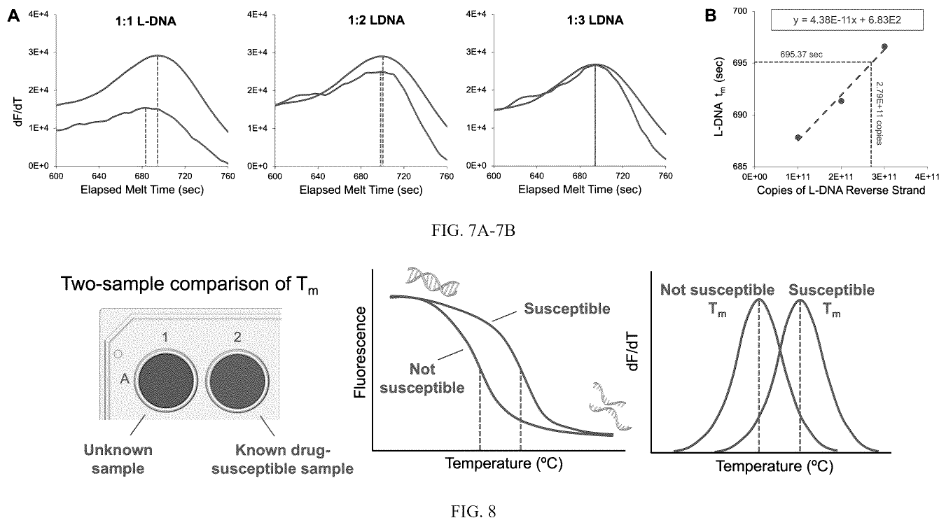

The standard HRM approach for drug susceptibility screening is based on a two-sample comparison of Tm's between an unknown PCR product and a known drug-susceptible PCR product (FIG. 8). Reactions were performed in the QuantStudio™ 5 real-time PCR thermal cycler (Thermo Fisher Scientific #A28137). This highly capable instrument was selected to facilitate standard HRM performance as a state-of-the-art comparison method for LHRM. Reactions had a 20 μL final volume containing 1× of SensiFAST™ Probe No-ROX Kit (Bioline #BIO-86005), 1×LCGreen® Plus (BioFire® Defense, LLC #BCHM-ASY-005), and 250 nM of each katG-specific primer (MEP176 and MEP177). Each target sample contained a final concentration of wild-type (MEP183) or mutant (MEP184-189,197-199) single-stranded DNA target at 2×10⁶ copies per reaction. An example of a standard HRM reaction setup is outlined in Table 3.

TABLE 3
Example reaction setup without L-DNA additive

| Component | [Stock] | [Final] | Volume (μL) |
|---|---|---|---|
| Nuclease-free water | — | — | 5 |
| SensiFAST ™ Probe | 2x | 1x | 10 |
| No-ROX Kit | | | |
| LCGreen ® Plus | 10x | 1x | 2 |
| Forward Primer | 10 μM | 250 nM | 0.5 |
| Reverse Primer | 10 μM | 250 nM | 0.5 |
| Target | — | — | 2 |
| | Total Volume | | 20 |

PCR reactions were initiated with a 95° C. hold for 2 min followed by 40 cycles of 95° C. for 5 sec and 59° C.¬ for 20 sec. A high resolution melt was performed immediately following PCR by annealing 95° C. to 50° C. at 0.1° C./sec followed by melting 65° C. to 95° C. at 0.025° C./sec (continuous acquisition mode). Double-stranded DNA PCR product fluorescence was monitored during PCR and during the melt reaction using LCGreen® Plus on the green optical channel (excitation 470±15/emission 520±15). Complete details are included herein.

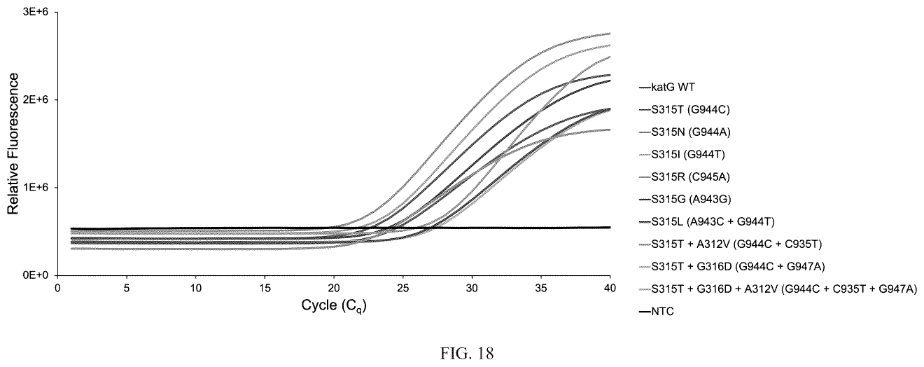

Standard HRM Analysis and Statistics PCR quantification cycle (Cq) was determined with the QuantStudio™ 5 Design and Analysis Software. Non-amplifying samples did not report Cq and were excluded from the data analysis. Amplifying samples with Cq over 35 were excluded from the data analysis because they did not achieve the PCR plateau phase. Representative PCR amplification curves of samples are included in FIG. 18. Tm was calculated with the proprietary QuantStudio™ 5 Design and Analysis Software based on the first derivative of fluorescence with respect to temperature. Based on Tm analysis of all samples, Tm cutoff points were established to maximize test specificity when classifying standard HRM analyzed samples as drug-susceptible or not. Specificity was maximized to decrease the false positive rate, i.e., decrease the misdiagnosis of variant samples as drug-susceptible. This maximized specificity strategy is often used for HRM classification of TB samples with drug resistance. Each test sample was individually classified. A sample was classified as drug-susceptible when PCR product Tm was within the drug-susceptible Tm cutoff range of 82.4° C. and 82.5° C. Since true positives are known, standard HRM was assessed for its sensitivity and specificity using this Tm cutoff range to classify drug susceptibility among 9 true drug-susceptible samples (n=3 trials of wild-type in triplicate) and 81 true not drug-susceptible samples (n=3 trials of 9 variant types in triplicate). In the experiment testing heating variability, significance was evaluated using Tm comparison (unpaired t test, significance level of α=0.95) of 96-well plate quadrants of S315T as compared to wild-type (n=1 trial with 24 replicates per sample type). All statistics were performed in Microsoft® Excel 2022 except for the sensitivity and specificity analysis that was performed in Python. Complete details are included herein.

### Standard HRM Approach, Statistics, and Analysis

### Approach

The standard HRM approach for drug susceptibility screening is based on a two-sample comparison of Tm's between an unknown PCR product and a known drug-susceptible PCR product (FIG. 8). Reactions were performed in the Applied Biosystems™ QuantStudio™ 5 real-time PCR thermal cycler (Thermo Fisher Scientific #A28137). This highly capable instrument was selected to facilitate standard HRM performance as a state-of-the-art comparison method for LHRM. QuantStudio™ 5 uses a 96-well format1 (Applied Biosystems™ #4483485). Reactions had a 20 μL final volume containing 1× of SensiFAST™ Probe No-ROX Kit (Bioline #BIO-86005), 1×LCGreen® Plus (BioFire® Defense, LLC #BCHM-ASY-005), and 250 nM of each katG-specific primer (MEP176 and MEP177). Each target sample contained a final concentration of wild-type (MEP183) or mutant (MEP184-189,197-199) single-stranded DNA target at 2×10⁶ copies per reaction. An example of a standard HRM reaction setup is outlined in Table 3. Samples were loaded into the 96-well plate such that each set of sample type triplicates were loaded into consecutive wells in the same row, except for experiments testing heating variability across the 96-well plate in which wild-type and S315T samples were loaded into mirrored quadrants of the 96-well plate. PCR reactions were initiated with a 95° C. hold for 2 min followed by 40 cycles of 95° C. for 5 sec and 59° C.¬ for 20 sec. Fluorescence was measured at the end of the annealing/extension step (59° C.). A high resolution melt was performed immediately following PCR by annealing 95° C. to 50° C. at 0.1° C./sec followed by melting 65° C. to 95° C. at 0.025° C./sec (continuous acquisition mode). This melting ramp rate is often used in QuantStudio™ 5 HRM mutation scanning. Double-stranded DNA PCR product fluorescence was monitored during PCR and during the melt reaction using LCGreen® Plus on the green optical channel (excitation 470±15/emission 520±15). The QuantStudio™ 5 was initially factory-calibrated for optical and thermal accuracy6. All standard calibration statuses (ROI/Uniformity, Background, Dyes) 6 were kept current. Custom dye calibration and custom melt curve calibration6 were performed for LCGreen® Plus.

### Analysis and Statistics

PCR quantification cycle (Cq) was determined with the QuantStudio™ 5 Design and Analysis Software. Non-amplifying samples did not report Cq and were excluded from the data analysis. Amplifying samples with Cq over 35 were excluded from the data analysis because they did not achieve the PCR plateau phase. Representative PCR amplification curves of samples are included in FIG. 18. Tm was calculated with the proprietary QuantStudio™ 5 Design and Analysis Software based on the first derivative of fluorescence with respect to temperature. Based on Tm analysis of all samples, Tm cutoff points were established to maximize test specificity when classifying standard HRM analyzed samples as drug-susceptible or not. Specificity was maximized to decrease the false positive rate, i.e., decrease the misdiagnosis of variant samples as drug-susceptible. This maximized specificity strategy is often used for HRM classification of TB samples with drug resistance. Each test sample was individually classified. A sample was classified as drug-susceptible when PCR product Tm was within the drug-susceptible Tm cutoff range of 82.4° C. and 82.5° C. Since true positives are known, standard HRM was assessed for its sensitivity and specificity using this Tm cutoff range to classify drug susceptibility among 9 true drug-susceptible samples (n=3 trials of wild-type in triplicate) and 81 true not drug-susceptible samples (n=3 trials of 9 variant types in triplicate). The true positive (sensitivity) rate was calculated as the percentage of drug-susceptible (+) test results out of all true wild-type (+) samples. The true negative (specificity) rate was calculated as the percentage of not drug-susceptible (−) test results out of all true variant (−) samples. In the experiment testing heating variability, significance was evaluated using Tm comparison (unpaired t test, significance level of α=0.95) of 96-well plate quadrants of S315T as compared to wild-type (n=1 trial with 24 replicates per sample type). All statistics were performed in Microsoft® Excel 2022 except for the sensitivity and specificity analysis that was performed in Python.

### LHRM Approach

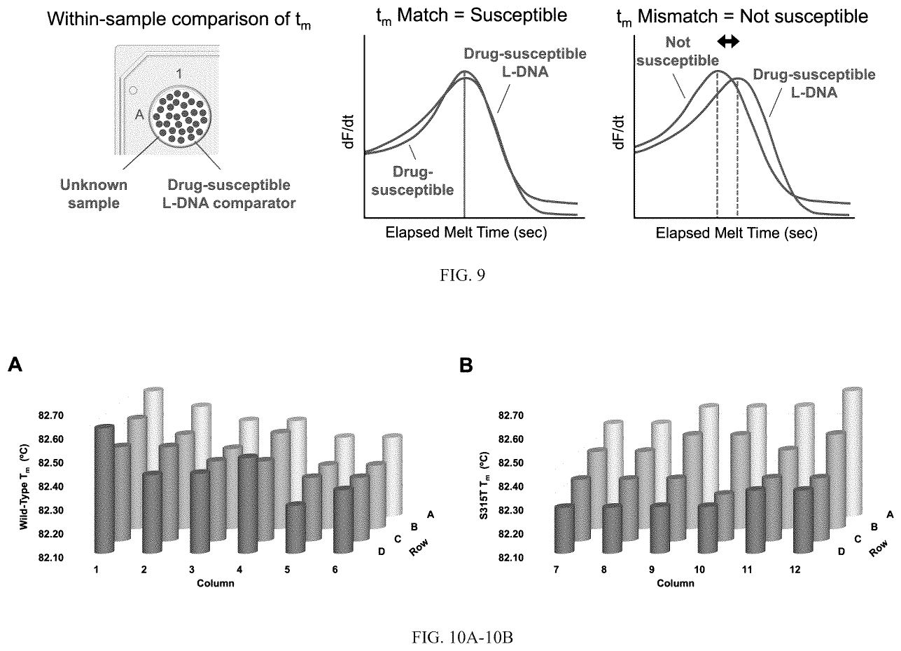

The methods as described herein, also referred to herein as “LHRM”, are used for drug susceptibility screening which is based on elapsed melt time (tm) comparison between an unknown PCR product and a drug-susceptible L-DNA comparator within a single sample (FIG. 9).

To ensure a fair comparison between the LHRM methods described herein and standard HRM approaches, both methods were tested using the same QuantStudio™ 5 instrument. LHRM used identical PCR cycling, PCR fluorescence monitoring, PCR quantification, melt reaction cycling, reaction loading placement, and heating variability test setup as standard HRM. LHRM statistics were identical to that of standard HRM, except for a data subset testing heating variability. Key changes from standard HRM are the inclusion of an additional reagent (L-DNA), monitoring melt reaction fluorescence on a second optical channel, and analysis of fluorescence changes as a function of time from the start of the QuantStudio™ 5 continuous mode melt instead of melt temperature provided by the instrument's calibration.

A double-stranded L-DNA drug-susceptible comparator was synthesized using left-helical enantiomeric DNA bases (i.e., L-DNA) with an identical sequence to the known drug-susceptible katG sequence. The 56-base L-DNA was synthesized with the same length and sequence as the drug-susceptible PCR amplicon. The double-stranded L-DNA was end-labeled with Texas Red (TXR) fluorophore and Black Hole Quencher 2 (BHQ2) quencher to monitor its behavior during melting on the orange fluorescence channel (excitation 580±10/emission 623±14). Detailed information on the L-DNA oligonucleotide sequences used herein are shown in Table 2. LHRM reactions included 2 μL of L-DNA mix with final copy counts of 1×10¹¹ copies TXR-labeled forward strand L-DNA (23FEB_katGf56_TXR) and 3×10¹¹ copies BHQ2-labeled reverse strand L-DNA (23FEB_katG_56_Rcmp+5_BHQ2) per reaction. An example reaction setup containing the L-DNA additive is outlined in Table 4.

TABLE 4
Example reaction setup containing L-DNA additive

| Component | [Stock] | [Final] | Volume (μL) |
|---|---|---|---|
| Nuclease-free water | — | — | 3 |
| SensiFAST ™ Probe | 2x | 1x | 10 |
| No-ROX Kit | | | |
| LCGreen ® Plus | 10x | 1x | 2 |
| Forward Primer | 10 μM | 250 nM | 0.5 |
| Reverse Primer | 10 μM | 250 nM | 0.5 |
| L-DNA mix | — | — | 2 |
| Target | — | — | 2 |
| | Total Volume | | 20 |

To ensure identical melt characteristics of D-DNA and end-labeled L-DNA, additional experiments were performed varying L-DNA strand concentration and strand ratio. In experiments varying L-DNA strand concentrations, reaction component deviations included 100 nM final concentration of each katG-specific primer and 1×10¹¹, 2×10¹¹, and 4×10¹¹ copies of L-DNA strands (forward and reverse) per reaction. In experiments varying L-DNA forward to reverse strand ratio, reaction component deviations included 100 nM final concentration of each katG-specific primer and 2 μL of L-DNA mix at 1:1, 1:2, and 1:3 ratios of forward to reverse strands for final L-DNA copy numbers of 1×10¹¹ copies of forward strand plus 1×10¹¹, 2×10¹¹, and 3×10¹¹ copies of reverse strand, respectively. Linear interpolation of three different L-DNA strand ratios was used to determine the relationship between copies of L-DNA reverse strands per reaction and L-DNA melt measurement. The L-DNA reverse strand copy number with a melt measurement matching that of wild-type PCR product was selected. Complete details are included herein.

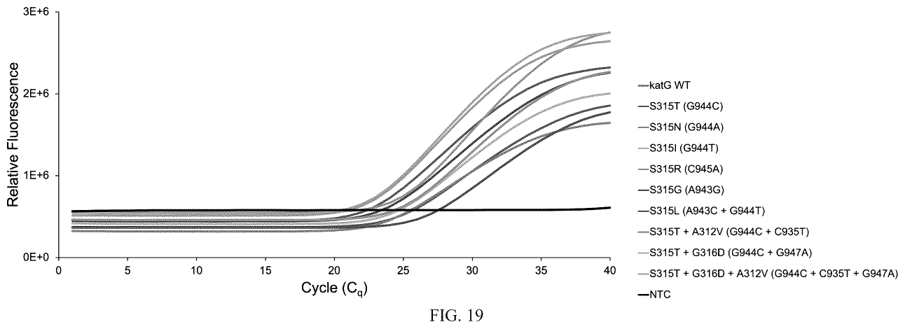

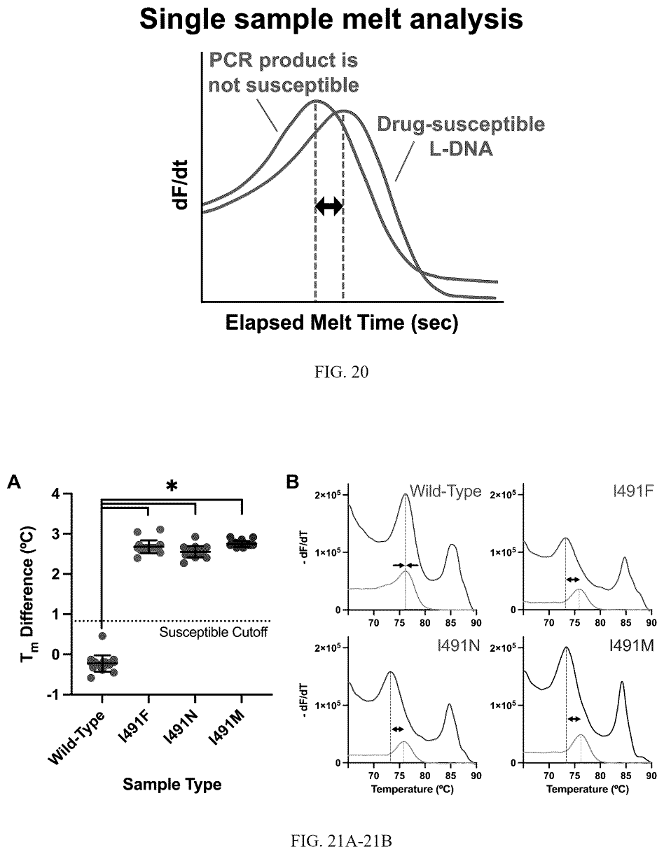

LHRM Analysis and Statistics Representative PCR amplification curves of samples containing L-DNA are included in FIG. 19. Elapsed melt time (tm) was calculated from the second degree Savitsky-Golay polynomials at each point (performed in MATLAB 2023A) based on the first derivative of fluorescence with respect to elapsed melt time. Elapsed melt time is a means of Tm reporting derived from the uncalibrated QuantStudio™ 5 raw data. Here, tm is defined as the elapsed melt time (in seconds) to reach the maximum derivative of fluorescence with respect to elapsed melt time. Significant differences between wild-type PCR product and L-DNA within each sample were assessed using paired t tests (of tm) with a significance level of α=0.95 (n=3 trials in triplicate). Test samples were classified as drug-susceptible when sample tm difference was zero. LHRM classification criteria is based on our assumption that L-DNA and PCR product melt characteristics are identical if and only if their sequences match. Specificity was maximized to decrease the false positive rate. Since true positives are known, LHRM was assessed for its sensitivity and specificity using a tm difference of zero to classify drug susceptibility among 9 true drug-susceptible samples (n=3 trials of wild-type in triplicate) and 79 true not drug-susceptible samples (n=3 trials of 9 variant types in triplicate, except variant S315T+G316D+A312V which had one trial with a single replicate due to Cq exclusion).

To directly compare time-based LHRM analysis within a single sample and temperature-based standard HRM analysis between samples, L-DNA-containing samples were also analyzed using standard HRM analysis. Sample Tm was calculated with the proprietary QuantStudio™ 5 Design and Analysis Software. Based on Tm analysis of all samples, Tm cutoff points were established to maximize test specificity when classifying each test sample as drug-susceptible or not. A sample was classified as drug-susceptible when PCR product Tm was within the drug-susceptible Tm cutoff range of 82.4° C. and 82.5° C. Since true positives are known, classification sensitivity and specificity were assessed using this Tm cutoff range to classify drug susceptibility among 9 true drug-susceptible samples (n=3 trials of wild-type in triplicate) and 79 true not drug-susceptible samples (n=3 trials of 9 variant types in triplicate, except variant S315T+G316D+A312V of one trial with a single replicate due to Cq exclusion).

Alternative strategies exist to establish drug-susceptible classification cutoff points for HRM analysis, and this was explored herein. This supplemental work used a maximized Youden J Statistic to establish drug-susceptible classification cutoff points for the same data sets across standard HRM and LHRM analysis strategies. This alternative cutoff strategy generally improved sensitivity and decreased specificity.

In the experiment testing heating variability, significance was evaluated using melt measurement comparison (Mann-Whitney U test, significance level of α=0.95) of 96-well plate quadrants of S315T as compared to wild-type (n=1 trial with 24 replicates). The heating variability Mann-Whitney U test was performed twice, once using Tm as the melt measurement and once using tm difference as the melt measurement. All statistics were performed in Microsoft® Excel 2022 except for the sensitivity and specificity analysis that was performed in Python. Complete details are included herein.

### LHRM Approach, Statistics, and Analysis

### Approach

LHRM for drug susceptibility screening is based on elapsed melt time (tm) comparison between an unknown PCR product and a drug-susceptible L-DNA comparator within a single sample (FIG. 9). To ensure a fair comparison between LHRM and standard HRM, both methods were tested using the same QuantStudio™ 5 instrument. LHRM used identical PCR cycling, PCR fluorescence monitoring, PCR quantification, melt reaction cycling, reaction loading placement, and heating variability test setup as standard HRM. LHRM statistics were identical to that of standard HRM, except for a data subset testing heating variability. Key changes from standard HRM are the inclusion of an additional reagent (L-DNA), monitoring melt reaction fluorescence on a second optical channel, and analysis of fluorescence changes as a function of time from the start of the QuantStudio™ 5 continuous mode melt instead of melt temperature provided by the instrument's calibration.

A double-stranded L-DNA drug-susceptible comparator was synthesized using left-helical enantiomeric DNA bases (i.e., L-DNA) with an identical sequence to the known drug-susceptible katG sequence. The 56-base L-DNA was synthesized with the same length and sequence as the drug-susceptible PCR amplicon. The double-stranded L-DNA was end-labeled with Texas Red (TXR) fluorophore and Black Hole Quencher 2 (BHQ2) quencher to monitor its behavior during melting on the orange fluorescence channel (excitation 580±10/emission 623±14). L-DNA fluorescence signal was scaled up by a factor of 18 in derivative melt plots for visual comparison with the PCR product's higher fluorescence signal. Detailed information on the L-DNA oligonucleotide sequences used in these studies are shown in Table 2. LHRM reactions included 2 μL of L-DNA mix with final copy counts of 1×10¹¹ copies TXR-labeled forward strand L-DNA (23FEB_katGf56_TXR) and 3×10¹¹ copies BHQ2-labeled reverse strand L-DNA (23FEB_katG_56_Rcmp+5_BHQ2) per reaction. An example reaction setup containing the L-DNA additive is outlined in Table 4.

To ensure identical melt characteristics of D-DNA and end-labeled L-DNA, additional experiments were performed varying L-DNA strand concentration and strand ratio. In experiments varying L-DNA strand concentrations, reaction component deviations included 100 nM final concentration of each katG-specific primer and 1×10¹¹, 2×10¹¹, and 4×10¹¹ copies of L-DNA strands (forward and reverse) per reaction. In experiments varying L-DNA forward to reverse strand ratio, reaction component deviations included 100 nM final concentration of each katG-specific primer and 2 μL of L-DNA mix at 1:1, 1:2, and 1:3 ratios of forward to reverse strands for final L-DNA copy numbers of 1×10¹¹ copies of forward strand plus 1×10¹¹, 2×10¹¹, and 3×10¹¹ copies of reverse strand, respectively. Linear interpolation of three different L-DNA strand ratios was used to determine the relationship between copies of L-DNA reverse strands per reaction and L-DNA melt measurement. The L-DNA reverse strand copy number with a melt measurement matching that of wild-type PCR product was selected.

### Analysis and Statistics

Representative PCR amplification curves of samples containing L-DNA are included in FIG. 19. Elapsed melt time (tm) was calculated from the second degree Savitsky-Golay polynomials at each point (performed in MATLAB 2023A) based on the first derivative of fluorescence with respect to elapsed melt time. Elapsed melt time is a means of Tm reporting derived from the uncalibrated QuantStudio™ 5 raw data. Here, tm is defined as the elapsed melt time (in seconds) to reach the maximum derivative of fluorescence with respect to elapsed melt time. Significant differences between wild-type PCR product and L-DNA within each sample were assessed using paired t tests (of tm) with a significance level of α=0.95 (n=3 trials in triplicate). Test samples were classified as drug-susceptible when sample tm difference was zero. LHRM classification criteria is based on our assumption that L-DNA and PCR product melt characteristics are identical if and only if their sequences match. Specificity was maximized to decrease the false positive rate, i.e., decrease the misdiagnosis of variant samples as drug susceptible. Since true positives are known, LHRM was assessed for its sensitivity and specificity using a tm difference of zero to classify drug susceptibility among 9 true drug-susceptible samples (n=3 trials of wild-type in triplicate) and 79 true not drug-susceptible samples (n=3 trials of 9 variant types in triplicate, except variant S315T+G316D+A312V which had one trial with a single replicate due to Cq exclusion).

To directly compare time-based LHRM analysis within a single sample and temperature-based standard HRM analysis between samples, L-DNA-containing samples were also analyzed using standard HRM analysis. Sample Tm was calculated with the proprietary QuantStudio™ 5 Design and Analysis Software. Based on Tm analysis of all samples, Tm cutoff points were established to maximize test specificity when classifying each test sample as drug-susceptible or not. Specificity was maximized to decrease the false positive rate. A sample was classified as drug-susceptible when PCR product Tm was within the drug-susceptible Tm cutoff range of 82.4° C. and 82.5° C. Since true positives are known, classification sensitivity and specificity were assessed using this Tm cutoff range to classify drug susceptibility among 9 true drug-susceptible samples (n=3 trials of wild-type in triplicate) and 79 true not drug-susceptible samples (n=3 trials of 9 variant types in triplicate, except variant S315T+G316D+A312V of one trial with a single replicate due to Cq exclusion).

Alternative strategies exist to establish drug-susceptible classification cutoff points for HRM analysis, and this was explored herein. This supplemental work used a maximized Youden J Statistic to establish drug-susceptible classification cutoff points for the same data sets across standard HRM and LHRM analysis strategies. This alternative cutoff strategy generally improved sensitivity and decreased specificity.

In the experiment testing heating variability, significance was evaluated using melt measurement comparison (Mann-Whitney U test, significance level of α=0.95) of 96-well plate quadrants of S315T as compared to wild-type (n=1 trial with 24 replicates). The heating variability Mann-Whitney U test was performed twice, once using Tm as the melt measurement and once using tm difference as the melt measurement. All statistics were performed in Microsoft® Excel 2022 except for the sensitivity and specificity analysis that was performed in Python.

### Results and Discussion

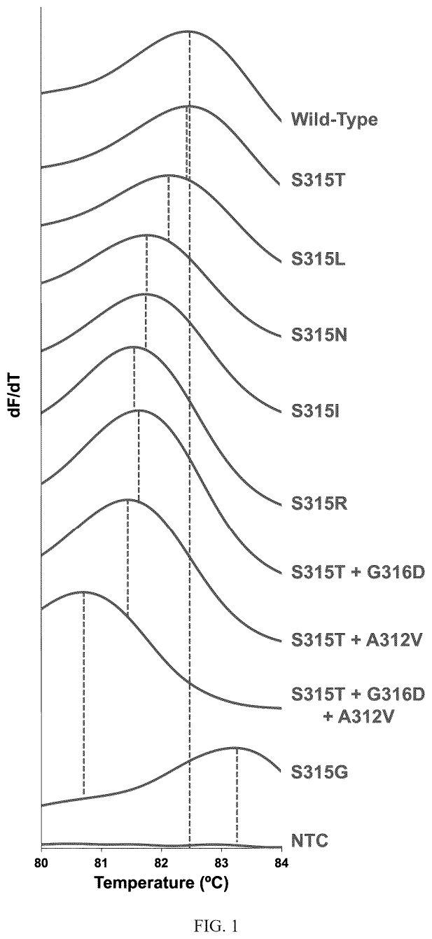

An initial classification of the test variants by standard HRM confirmed that classification was successful for most variants but that the small melt difference in the single base mutation S315T was near the limits of this calibrated instrument approach. As predicted by the theoretical melt differences (Table 1), all variant samples analyzed by standard HRM had lower melt temperatures compared to the known wild-type samples, except S315G which had a higher melt temperature (FIG. 1). The nine selected variants had a Tm spread of 2.43° C., offering both easy and challenging classification cases against wild-type (FIG. 1). Average sample Tm's and Tm differences are reported in Table 5.

TABLE 5
Summary of standard HRM Tm's and Tm differences of katG wild-type and
nine katG variants (n = 3 trials in triplicate per sample type)

| | | | Average Tm Difference |
|---|---|---|---|
| | | Average PCR | from Global Average |
| | | Product Tm | Known Wild-Type |
| katG Sample Type | NA Base Change | (° C. ± SD) | (° C. ± SD) |
| Wild-Type | None | 82.47 ± 0.06 | 0.00 ± 0.06 |
| S315T | G944C | 82.29 ± 0.13 | −0.18 ± 0.13 |
| S315N | G944A | 81.75 ± 0.03 | −0.70 ± 0.03 |
| S315I | G944T | 81.71 ± 0.08 | −0.75 ± 0.08 |
| S315R | C945A | 81.58 ± 0.08 | −0.88 ± 0.08 |
| S315G | A943G | 83.06 ± 0.14 | 0.61 ± 0.14 |
| S315L | A943C + G944T | 82.12 ± 0.03 | −0.33 ± 0.03 |
| S315T + A312V | G944C + C935T | 81.43 ± 0.07 | −1.02 ± 0.07 |
| S315T + G316D | G944C + G947A | 81.64 ± 0.08 | −0.81 ± 0.08 |
| S315T + G316D + A312V | G944C + G947A + C935T | 80.63 ± 0.14 | −1.82 ± 0.14 |

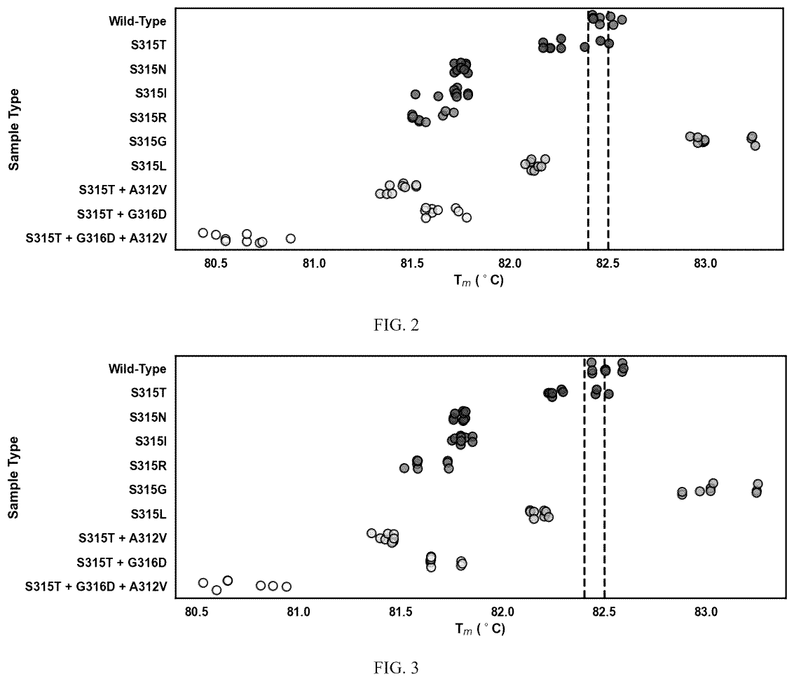

Standard HRM correctly classified 6/9 wild-type katG samples as drug-susceptible and 80/81 variant samples as not drug-susceptible; the most clinically prevalent variant S315T was misclassified once. Although S315G has the smallest theoretical melt difference (0.33° C.) from wild-type and was initially thought to be the most difficult variant to correctly classify, S315T was experimentally the most difficult case because under the sample salt conditions it induced the smallest melt difference (−0.18° C.) from wild-type among all nine variants. Standard HRM relied on Tm analysis of data from multiple samples to form the drug-susceptible classification Tm cutoff range of 82.4° C. to 82.5° C. Using a state-of-the-art calibrated instrument, standard HRM performed at 66.7% sensitivity and 98.8% specificity when classifying drug susceptibility. Sample classification accuracy and relationships between sample type Tm's are shown in FIG. 2. In particular, standard HRM misclassification is illustrated by three wild-type samples above the upper drug-susceptible cutoff range and one S315T sample within the drug-susceptible cutoff range.

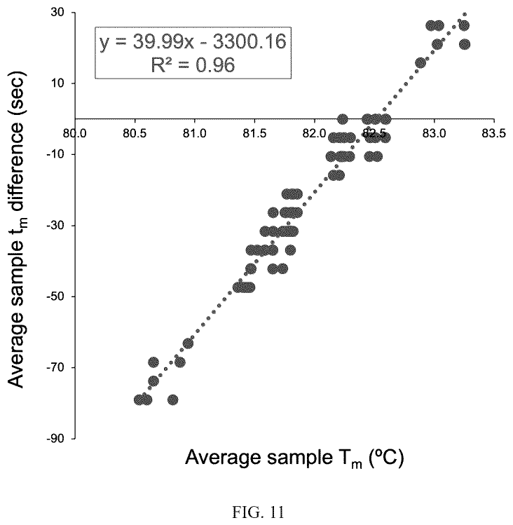

This S315T classification error was thought to be due to instrument heating variability that has been observed in many plate-based real-time PCR instruments. Non-uniform heating limits Tm comparison accuracy and is only partially compensated for by instrument calibration. In a direct test of this, the QuantStudio™ 5 was found to exhibit heating variability across the 96-well plate (FIGS. 10A-10B) and this impacted standard HRM classification of the most difficult case, S315T. Standard HRM failed to distinguish between wild-type and S315T when using PCR product melt characteristics since there was no significant difference between wild-type and S315T sample Tm's (p>0.05, unpaired t test, 24 replicates per sample type). The 24 identical samples exhibited thermal edge effects of generally higher Tm's and zone-based Tm's for two samples in shared heating elements, with trends holding for both wild-type and S315T sample types (FIG. 10A-10B). Specifically, wild-type average PCR product Tm (mean±SD) was 82.46±0.08° C. across all wells and 82.53±0.07° C. across edge wells. S315T average PCR product Tm (mean±SD) was 82.42±0.10° C. across all wells and 82.50±0.09° C. across edge wells. The heating variability of the QuantStudio™ 5 (FIGS. 10A-10B) is presumably due to variations in the instrument's six independent temperature heating zones (pairwise vertical columns) that are not completely corrected by the instrument calibration procedures.

We used this simple experimental design to test the initial feasibility of the L-DNA approach. Repeating this heating experiment but incorporating L-DNA into each sample did not change the experimental outcome. The approach still failed to distinguish between wild-type and S315T because there was no statistical difference between PCR product Tm's (p>0.05, Mann-Whitney U test, 24 replicates per sample type, column 2 in Table 8). These results confirmed that the addition of L-DNA itself does not affect the between-sample analysis based on temperature and indirectly supports the initial assumption that L-DNA does not interfere with the PCR reaction. However, re-analysis using PCR product to L-DNA melt time differences did successfully overcome effects from non-uniform heating and distinguished between variant S315T and wild-type. The additional L-DNA elapsed time data in this experimental design was used to show that a re-analysis incorporating a within-sample melt difference between L-DNA and PCR product distinguishes between these two sequences with a single base difference. Using tm differences, there was a significant difference between quadrants of wild-type and S315T samples (p<0.05, Mann-Whitney U test, 24 replicates per sample type, column 4 in Table 8).

Due to the success of the limited data set employing L-DNA, the full variant test bed (Table 1) previously performed (FIG. 2) was run incorporating an L-DNA comparator into every sample. These data were analyzed by two different methods: standard HRM analysis using temperature-based melt characteristics between PCR products of multiple samples and LHRM analysis using time-based melt differences between the PCR product and L-DNA comparator within each sample.

As shown in FIG. 3, standard HRM analysis classification accuracy of L-DNA-containing samples was similar to the original standard HRM experiment without L-DNA (FIG. 2). Average sample Tm's and Tm differences are reported in Table 6.

TABLE 6
Summary of PCR product Tm's and Tm differences of samples containing an L-DNA
comparator in every sample and analyzed by standard HRM across wild-type and
nine variants (n = 3 trials in triplicate per sample type, except variant
S315T + G316D + A312V of one trial with a single replicate due to Cq exclusion)

| | | | Average Tm Difference |
|---|---|---|---|
| | | Average PCR | from Global Average |
| | | Product Tm | Known Wild-Type |
| katG Sample Type | NA Base Change | (° C. ± SD) | (° C. ± SD) |
| Wild-Type | None | 82.51 ± 0.07 | 0.00 ± 0.07 |
| S315T | G944C | 82.33 ± 0.12 | −0.18 ± 0.12 |
| S315N | G944A | 81.80 ± 0.03 | −0.72 ± 0.03 |
| S315I | G944T | 81.80 ± 0.03 | −0.71 ± 0.03 |
| S315R | C945A | 81.63 ± 0.08 | −0.89 ± 0.08 |
| S315G | A943G | 83.06 ± 0.15 | 0.55 ± 0.15 |
| S315L | A943C + G944T | 82.18 ± 0.04 | −0.33 ± 0.04 |
| S315T + A312V | G944C + C935T | 81.44 ± 0.04 | −1.07 ± 0.04 |
| S315T + G316D | G944C + G947A | 81.70 ± 0.07 | −0.81 ± 0.07 |
| S315T + G316D + A312V | G944C + G947A + C935T | 80.72 ± 0.15 | −1.79 ± 0.15 |

3/9 wild-type katG samples were correctly classified as drug-susceptible and 77/79 variant samples were correctly classified as not drug-susceptible. Standard HRM misclassification is illustrated by six wild-type samples above the upper drug-susceptible cutoff range and two S315T samples within the drug-susceptible cutoff range (FIG. 3). Standard HRM analysis of L-DNA-containing samples performed at 33.3% sensitivity and 97.5% specificity when classifying drug susceptibility. Samples containing L-DNA and analyzed by standard HRM had decreased sensitivity and comparable specificity metrics as compared to samples without L-DNA and analyzed by standard HRM.

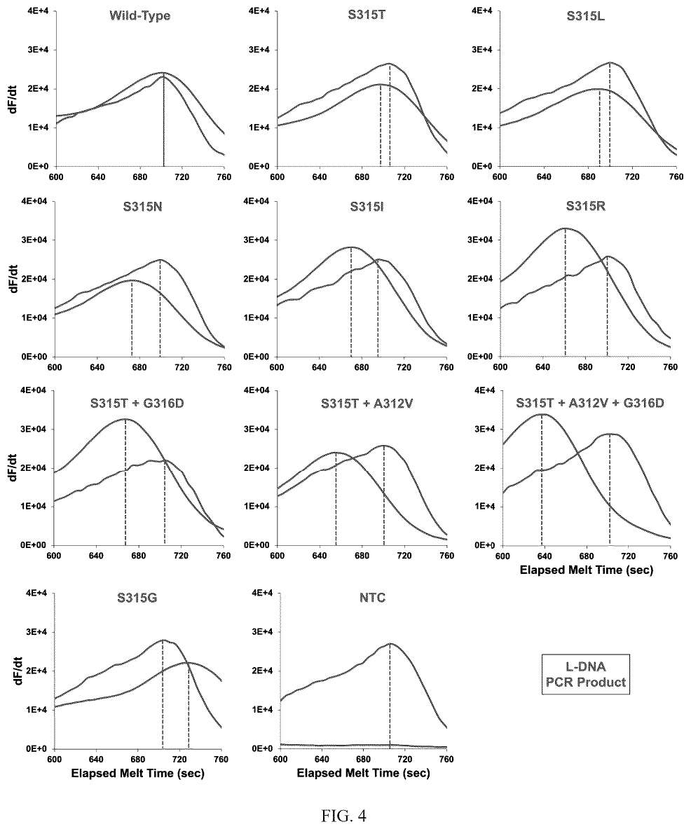

Samples containing L-DNA were then re-analyzed via LHRM analysis. The LHRM and standard HRM analysis strategies resulted in similar melt behavior across the wild-type and full variant test bed (FIG. 11). Using LHRM, all variant samples had lower elapsed melt times compared to the drug-susceptible comparator, except S315G which had a higher elapsed melt time (FIG. 4). L-DNA and DNA PCR product had nearly identical melt characteristics when the sequences matched (wild-type katG) but differed if there was a sequence mismatch (katG variants) (FIG. 4 and Table 7).

TABLE 7
Summary of LHRM tm's and tm differences of wild-type and nine variants
(n = 3 trials in triplicate per sample type, except variant S315T + G316D +
A312V which had one trial with a single replicate due to Cq exclusion)

| | | | | Average tm |
|---|---|---|---|---|
| | | Average PCR | Average | Difference from |
| | | Product Tm | L-DNA tm | L-DNA Within Well |
| katG Sample Type | NA Base Change | (sec ± SD) | (sec ± SD) | (sec ± SD) |
| Wild-Type | None | 700.79 ± 1.77 | 701.96 ± 2.63 | −1.17 ± 2.32 |
| S315T | G944C | 692.60 ± 3.84 | 700.20 ± 5.24 | −7.60 ± 3.82 |
| S315N | G944A | 673.30 ± 1.74 | 699.03 ± 4.42 | −25.74 ± 4.11 |
| S315I | G944T | 671.54 ± 2.80 | 699.03 ± 2.34 | −27.49 ± 3.51 |
| S315R | C945A | 664.52 ± 2.34 | 700.20 ± 2.60 | −35.68 ± 3.51 |
| S315G | A943G | 722.43 ± 5.15 | 700.79 ± 4.10 | 21.64 ± 4.12 |
| S315L | A943C + G944T | 689.09 ± 1.74 | 699.62 ± 3.15 | −10.53 ± 3.72 |
| S315T + A312V | G944C + C935T | 657.50 ± 1.79 | 702.54 ± 3.83 | −45.04 ± 3.82 |
| S315T + G316D | G944C + G947A | 667.45 ± 3.52 | 701.96 ± 4.56 | −34.51 ± 4.64 |
| S315T + G316D + A312V | G944C + G947A + C935T | 634.37 ± 0.09 | 702.81 ± 3.82 | −70.20 ± 8.05 |

TABLE 8
Summary of melt characteristics for 24 identical wild-type samples
containing L-DNA and 24 identical S315T samples containing L-DNA
in the top left and top right quadrants, respectively, of 96-well
heating block (n = 1 trial). Samples were analyzed by standard
HRM analysis for PCR product Tm's and analyzed by LHRM analysis
for PCR product tm's and tm differences. L-DNA dynamically calibrated
to heating variability, enabling wild-type and S315T samples to be
successfully differentiated using within-sample tm differences

| | | | Average tm |
|---|---|---|---|
| | Average PCR | Average PCR | Difference (from |
| katG | Product Tm | Product tm | L-DNA Within Well) |
| Sample | Across All Wells | Across All Wells | Across All Wells |
| Type | (° C. ± SD) | (sec ± SD) | (sec ± SD) |
| Wild-Type | 82.45 ± 0.09 | 697.90 ± 3.79 | 0.66 ± 4.50 |
| S315T | 82.43 ± 0.10 | 697.23 ± 3.81 | −4.62 ± 3.24 |

Using melt time differences between PCR product and L-DNA within each sample, LHRM correctly classified 7/9 wild-type katG samples as drug-susceptible and 78/79 variant samples as not drug-susceptible.

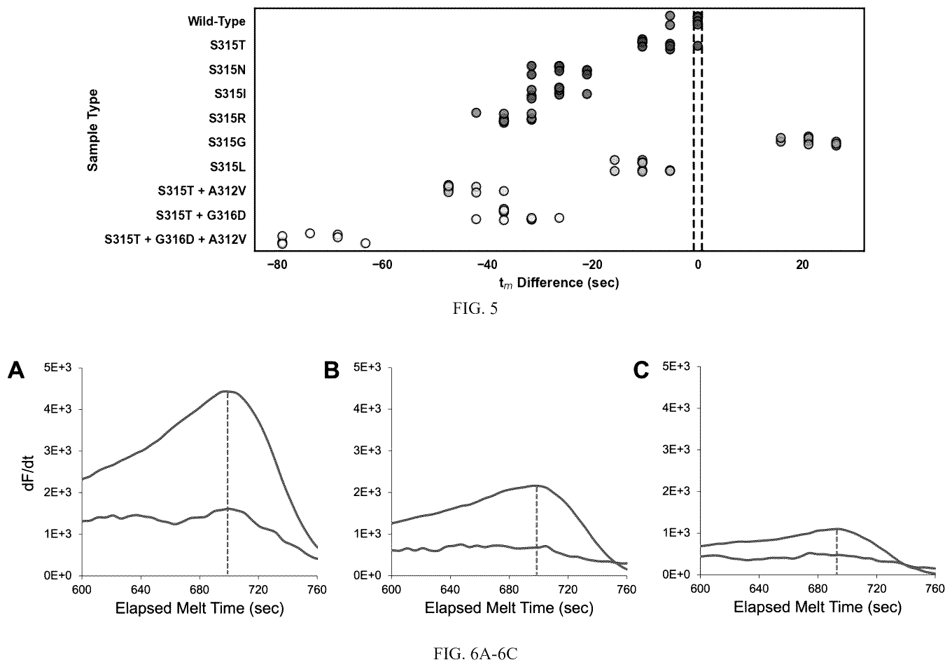

Unlike standard HRM, LHRM drug-susceptible classification criteria were established without relying on analysis of data from multiple samples. Instead, LHRM classified samples as drug-susceptible when a sample's tm difference was zero. This simple classification strategy enabled single sample classification without requiring data from other samples. LHRM performed at 77.8% sensitivity and 98.7% specificity when classifying drug susceptibility. Sample classification accuracy and relationships between sample type tm differences are illustrated in FIG. 5, which can be directly compared to FIG. 3. In particular, LHRM misclassification is illustrated by two wild-type samples below the drug-susceptible cutoff and one S315T sample within the drug-susceptible cutoff (FIG. 5). S315T was the only variant misclassified by LHRM analysis, as similarly observed in standard HRM analysis classification (FIG. 3). LHRM correctly classified drug susceptibility with specificity very similar to and with improved sensitivity over standard HRM analyzed samples. This trend held for LHRM as compared to standard analyzed samples with and without L-DNA. Notably, LHRM accomplished high success metrics without requiring multiple sample classification data and without relying on temperature-based melt reporting determined by instrument calibration.

Heating variation was identified as a major methodological artifact that reduced classification performance for S315T by HRM (FIG. 10A-10B). While no other methodological artifacts were identified in HRM testing, it was speculated that some types of sample preparation errors could introduce systematic hybridization changes that could also be corrected using L-DNA. Possible errors may include culture media carryover, extraction errors, kit-to-kit master mix differences, or sample-to-sample salt concentration variability resulting from reagent pipetting errors. A contrived study of salt differences is included herein to demonstrate an example of how LHRM can correct for these types of errors. In particular, this supplemental study verified that a sample's salt content has a detrimental potential impact on classification accuracy and that within-assay differences can be overcome by LHRM to ultimately restore classification capabilities.

Three key features are critical for the success of the LHRM approach, including but not limited to:

(1) LHRM reactions must include the same amount of double-stranded L-DNA in every sample to provide a signature comparator hybridization event. Constant L-DNA concentration in every sample was maintained by adding L-DNA from a stock into the master mix. Since L-DNA and the PCR product hybridization events were changed by heating (FIGS. 10A-10B) and salt variation errors in the same way, the L-DNA to PCR product melt difference corrected for any artifact-induced melt shifts. Small mutation-induced melt shifts in the PCR product were then detectable due to the reduction in between-sample errors and ultimately facilitated LHRM classification performance with specificity very similar to and improved sensitivity over the standard method.

(2) Determining how to discriminate between double-stranded L-DNA and D-DNA melting behavior in a single sample. Apparently, all readily available intercalating dyes do not discriminate between enantiomeric DNA (Table 10). Therefore, if double-stranded L-DNA and double-stranded D-DNA are combined in a single reaction, the intercalation melt signal reports a composite melt curve. To overcome this L-DNA and D-DNA intercalating crosstalk, L-DNA was end-labeled with Texas Red. The goal was to measure only double-stranded DNA PCR product fluorescence signal on the green optical channel using LCGreen® intercalating dye and measure only double-stranded L-DNA fluorescence signal on the orange optical channel using Texas Red fluorophore and quencher end-labeling. FIG. 6A demonstrates intercalating crosstalk (i.e., detectable melt signals on both fluorophore-quencher and intercalator channels) when samples contained 4×10¹¹ copies of double-stranded L-DNA. Samples with 1×10¹¹ copies of double-stranded L-DNA (FIG. 6C) produced sufficient fluorophore-quencher signal for accurate L-DNA melt measurements with little crosstalk contribution detectable in the intercalator channel, and therefore, this L-DNA copy count was selected for further LHRM development.

(3) Matching the melt characteristics of the L-DNA melt comparator to the melt characteristics of the wild-type PCR product to support the initial melt matching assumption. Several factors made matching the melt characteristics of the two difficult. It is well known that the L-DNA sequence end-labeling used to overcome single tube detection also changes the DNA's melt temperature, even with a 5-base spacer on the quencher strand. Previous reports have also established that total DNA concentration and strand ratio affect the melt temperature. In addition to these well-known factors, even when they have the same sequence, for unknown reasons, unlabeled double-stranded L-DNA and D-DNA have a small difference in melt temperatures measured by intercalation (Table 10). Since strand ratio was the easiest to adjust, the strand ratio of the added L-DNA was empirically modified to compensate for these other factors and ultimately match the L-DNA and D-DNA wild-type melt characteristics. The theory behind this report's experimental tuning strategy is detailed in a complementary work, Spurlock et al. 2024, incorporated herein by reference for all purposes. Spurlock et al. 2024 details the theory behind the effects of DNA concentration and strand ratio on annealing while this report experimentally demonstrates its use in melt analysis. As FIG. 7A demonstrates, different L-DNA forward-to-reverse strand ratios shift L-DNA tm (Table 9).

TABLE 9
Summary of D-DNA tm's, L-DNA tm's, and associated tm differences
for LHRM analyzed samples with double-stranded L-DNA at 1:1,
1:2, and 1:3 ratio of forward to reverse L-DNA strands (1 × 10¹¹
forward strand copies and 1 × 10¹¹, 2 × 10¹¹, and 3 × 10¹¹
reverse strand copies per reaction, respectively). L-DNA
forward-to-reverse strand ratios shifted L-DNA tm.

| Ratio of Forward | Average | Average | Average |
|---|---|---|---|
| to Reverse | D-DNA tm | L-DNA tm | tm Difference |
| L-DNA Strands | (sec ± SD) | (sec ± SD) | (sec ± SD) |
| 1:1 | 696.60 ± 3.04 | 687.83 ± 10.96 | 8.77 ± 8.04 |
| 1:2 | 694.85 ± 5.26 | 691.34 ± 8.04 | 3.51 ± 3.04 |
| 1:3 | 694.85 ± 0.00 | 96.60 ± 3.04 | −1.75 ± 3.04 |

TABLE 10
Summary of Tm's and Tm differences for 1:1 D-DNA and 1:1 unlabeled
L-DNA using three different intercalating dyes (n = 1
trial in triplicate per sample type per intercalating dye).
Unlabeled double-stranded L-DNA and D-DNA have small differences
in melt temperatures measured by intercalation. The intercalating
dyes did not discriminate between enantiomeric DNA.

| | Average | Average | Average Tm |
|---|---|---|---|
| | D-DNA Tm | L-DNA Tm | Difference |
| Intercalating Dye | (° C. ± SD) | (° C. ± SD) | (° C. ± SD) |
| EvaGreen ® | 82.81 ± 0.14 | 82.44 ± 0.00 | 0.38 ± 0.14 |
| EvaGreen ® Plus | 83.00 ± 0.08 | 82.72 ± 0.00 | 0.28 ± 0.08 |
| LCGreen ® Plus | 82.81 ± 0.00 | 82.72 ± 0.00 | 0.09 ± 0.00 |

TABLE 11
Oligonucleotide sequences designed for direct comparison of 1:1 L-DNA and unlabeled
1:1 D-DNA. DNA is denoted as D-DNA or L-DNA

| | DNA | | |
|---|---|---|---|
| ID | Type | Description | Sequence (5′ > 3′) |
| MEP213 | D-DNA | Wild-type drug- | (SEQ ID NO: 26) |
| | | susceptible | CACCGGAACCGGTAAGGACGCGATCACCAGCGGCATCGA |
| | | amplicon forward | GGTCGTATGGACGAACA |
| | | strand | |
| MEP214 | D-DNA | Wild-type drug- | (SEQ ID NO: 27) |
| | | susceptible | TGTTCGTCCATACGACCTCGATGCCGCTGGTGATCGCGTCC |
| | | amplicon reverse | TTACCGGTTCCGGTG |
| | | strand | |
| 23FEB_ | L-DNA | Wild-type drug- | (SEQ ID NO: 28) |
| katGf56 | | susceptible | CACCGGAACCGGTAAGGACGCGATCACCAGCGGCATCGA |
| | | internal | GGTCGTATGGACGAACA |
| | | comparator | |
| | | forward strand | |
| | | with end-labeling | |
| 23FEB_ | L-DNA | Wild-type drug- | (SEQ ID NO: 29) |
| katGRcmp56 | | susceptible | TGTTCGTCCATACGACCTCGATGCCGCTGGTGATCGCGTCC |
| | | internal | TTACCGGTTCCGGTG |
| | | comparator | |
| | | reverse strand | |
| | | with end-labeling | |

This phenomenon was used to compensate for all factors discussed above and achieve an empirical melt match between drug-susceptible L-DNA and D-DNA (right-most panel in FIG. 7A). As reverse strand L-DNA copy count increased, L-DNA tm increased (FIG. 7A and Table 9). A positive linear relationship between the number of L-DNA reverse strand copies per reaction and L-DNA tm indicated that 2.79×10¹¹ L-DNA reverse strand copies (and 1×10¹¹ L-DNA forward strand copies) per reaction would produce the 695 sec tm matching average wild-type PCR product tm. (FIG. 7B). The optimal L-DNA strand ratio was rounded up from 1:2.79 to 1:3 (1×10¹¹ L-DNA forward strand copies and 3×10¹¹ L-DNA reverse strand copies per reaction) for ease of sample preparation in LHRM. This method produced an average melt difference of approximately 1 sec between drug-susceptible L-DNA and wild-type PCR product (top row in Table 7). In further support of melt matching, there was no statistical difference between tm's of drug-susceptible L-DNA and wild-type PCR product (p>0.05, paired t test, n=3 trials in triplicate). It is important to note that although this study sought to match the L-DNA melt and PCR product melt, it is not critical to do so. Even without tuning L-DNA to make the melt difference zero, for a fixed concentration of L-DNA in every sample, the tm difference will still be a constant in the system and samples can be classified as drug-susceptible when sample tm difference equals that constant. Alternatively, intentionally tuning for an excessively large melt mismatch between L-DNA and the wild-type PCR product may help to minimize intercalating crosstalk.

L-DNA tuning and time-scale melt analysis contribute to the simplicity of LHRM single sample classification, i.e., a sample is susceptible if its tm difference equals zero. A given LHRM sample's tm difference can only consist of discrete values in multiples of 5.27 sec. Discrete melt data every 5.27 sec enables easier LHRM classification as melt data is inherently grouped into distinct time points and will clearly have tm differences of zero or not (FIG. 5). The discrete nature of LHRM melt data is an artifact of the instrument melting ramp rate of 0.025° C./sec and the fluorescence sampling rate of one acquisition per 5.27 sec. In contrast to discrete LHRM melt data, sample Tm's resulting from standard HRM analysis are continuous in nature. As a result, standard HRM analysis produces a spread of data points with less separation between sample types (FIG. 2 and FIG. 3). Unlike LHRM's discrete melt data, this continuous spread of data requires grouping into subsets using multiple samples to form the drug-susceptible classification cutoffs.

In this initial LHRM report, a QuantStudio™ 5 was used to show that LHRM can provide similar performance to standard HRM with use of a single sample. It is important to note, however, that the instrument itself is not critical for the conclusions of this work. Although not confirmed in this report, some features of LHRM suggest this strategy would work with less capable instrument designs. For example, LHRM does not use temperature-based melt reporting determined by instrument calibration. Instead, LHRM utilizes time-defined melt analysis. Measurements were quantified using time instead of temperature for two reasons. First, time-quantified melt measurements are based on raw data unaffected by instrument calibration errors present in the Tm. Secondly, this strategy facilitates future LHRM measurements in other types of real-time PCR instruments that are not as well-calibrated as the QuantStudio™ 5 instrument. To use an available real-time PCR instrument, it must at a minimum have melt analysis capabilities built into its software. Although existing real-time PCR instruments commonly have melt analysis capabilities for product assessment purposes, the instruments generally lack the necessary resolution for high sensitivity SNP scanning by melting. Performing LHRM on a real-time PCR instrument requires access to sample times and fluorescence values recorded during heating of the melt procedure. The resolution of LHRM classification within a single sample is still limited by correct differentiation between an L-DNA comparator sequence and a PCR product sequence that can differ by only a single base. Among the many factors that make this differentiation challenging is the ratio of the fluorescence sampling rate compared to the instrument's heating rate. A higher ratio makes differentiation easier. In this report, the ratio is 7.59 fluorescent samples collected per ° C. Since it is relatively easy to set the instrument's desired continuous ramp rate, the maximum sampling rate of the instrument is likely the determining factor that limits the resolution of this within-sample method. Future work is required to determine if the L-DNA reagent-based strategy can be implemented on other instruments with real-time PCR and built-in melt analysis data generation capabilities.

### Establishing Drug-Susceptible Classification Cutoff Points Using a Maximized Youden J Statistic

In this report, the standard HRM analysis strategy established drug-susceptible classification cutoff points to maximize specificity when classifying samples as drug-susceptible or not. In the context of drug-susceptibility testing, specificity was maximized to decrease the misdiagnosis of variant samples as drug-susceptible. Unlike standard HRM, LHRM drug-susceptible classification criteria were established without relying on analysis of data from multiple samples. LHRM classified samples as drug-susceptible when a sample's tm difference was zero.

Alternative strategies exist to establish drug-susceptible classification cutoff points for HRM analysis. In this supplemental analysis, a maximized Youden J Statistic was used to establish the drug-susceptible classification cutoff points for both standard HRM and LHRM analysis strategies. By maximizing the Youden J Statistic, which is symmetric in sensitivity and specificity, equal weight is given to false positives and false negatives. This “best overall” approach has trade-offs in the context of drug-susceptibility testing as it prioritizes equally the correct classification of true drug-susceptible cases and true not drug-susceptible cases. The Youden J Statistic requires data from multiple samples to inform classification of a single sample, whether applied to standard HRM or LHRM analysis. Across data sets analyzed by either analysis strategy, each test sample was individually classified. True positive (sensitivity) rate was calculated as the percentage of drug-susceptible (+) test results out of all true wild-type (+) samples. The true negative (specificity) rate was calculated as the percentage of not drug-susceptible (−) test results out of all true variant (−) samples. All Sensitivity and specificity analysis was performed in Python.

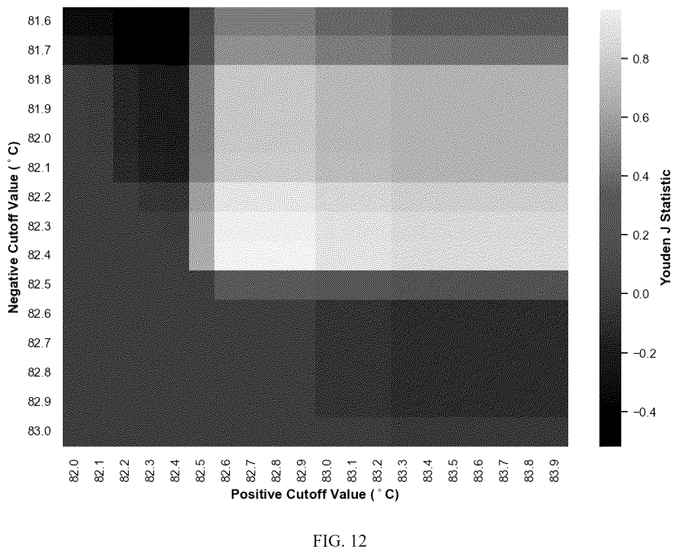

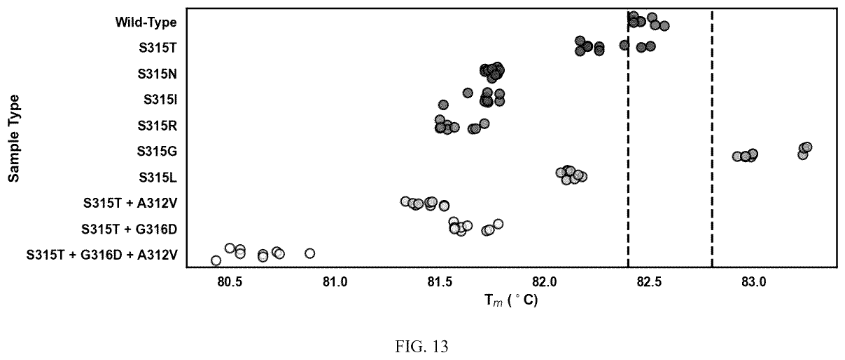

The Youden J Statistic was utilized to determine the sensitivity and specificity of samples without L-DNA and analyzed by standard HRM analysis. The Youden J Statistic was calculated for upper and lower bound drug-susceptible Tm cutoff values (in ° C.) and plotted as a heatmap (FIG. 12). The maximized Youden J Statistic in the heatmap established Tm cutoff values for standard HRM sample classification as drug-susceptible or not. This strategy is often used for HRM classification of TB samples with drug-resistance. A sample was classified as drug-susceptible when PCR product Tm was within the drug-susceptible Tm cutoff range of 82.4° C. and 82.8° C. Since true positives are known, the sample set was assessed for sensitivity and specificity using this Tm cutoff range when classifying drug-susceptibility among 9 true drug-susceptible samples (n=3 trials of wild-type in triplicate) and 81 true not drug-susceptible samples (n=3 trials of 9 variant types in triplicate). Using a maximized Youden J Statistic, samples analyzed by standard HRM performed at 100% sensitivity and 97.5% specificity. As compared to standard HRM metrics produced using a maximized specificity in this report, Youden-based metrics increased sensitivity by 33.3% but decreased specificity by 1.3%. Youden-based sample classification accuracy is illustrated in FIG. 13. In particular, standard HRM misclassification is illustrated by two S315T samples within the drug-susceptible cutoff range (FIG. 13).

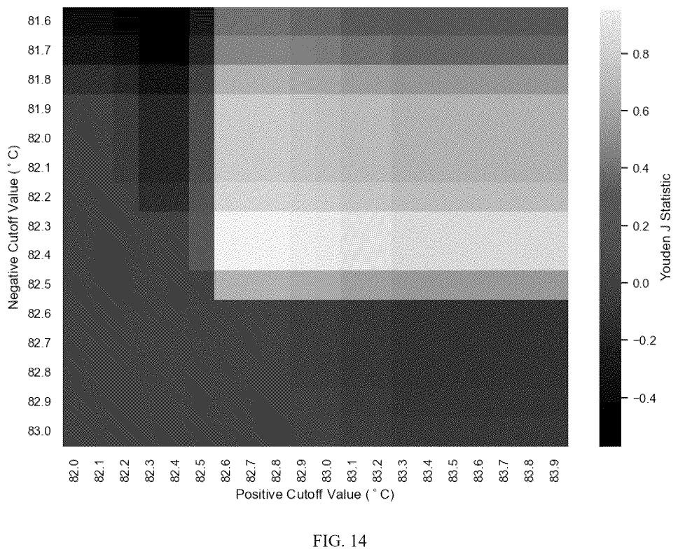

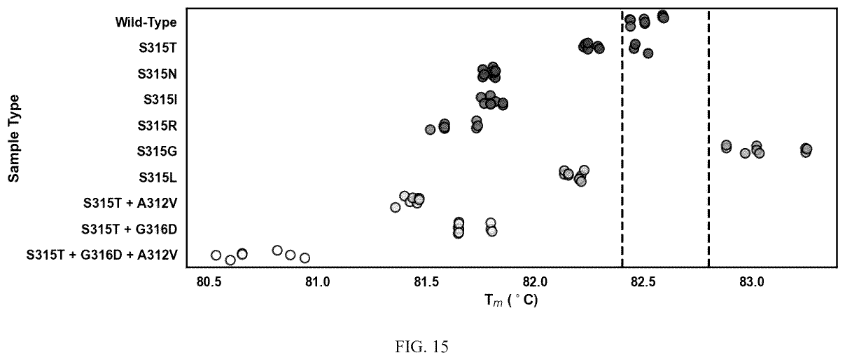

The Youden J Statistic was utilized to determine the sensitivity and specificity of samples containing L-DNA but analyzed by standard HRM analysis. The Youden J Statistic was calculated for upper and lower bound drug-susceptible cutoff values and plotted as a heatmap (FIG. 14). The maximized Youden J Statistic in the heatmap established Tm cutoff values for sample classification as drug-susceptible or not. A sample was classified as drug-susceptible when sample Tm was within the drug-susceptible Tm cutoff range of 82.4° C. to 82.8° C. Notably, this Youden-based cutoff range was the same for standard HRM analysis of samples with L-DNA (FIG. 15) and without L-DNA (FIG. 13). Since true positives are known, the sample set was assessed for sensitivity and specificity using this Tm cutoff range when classifying drug-susceptibility among 9 true drug-susceptible samples (n=3 trials of wild-type in triplicate) and 79 true not drug-susceptible samples (n=3 trials of 9 variant types in triplicate, except variant S315T+G316D+A312V of one trial with a single replicate due to Cq exclusion). Using a maximized Youden J Statistic, L-DNA-containing samples analyzed by standard HRM performed at 100% sensitivity and 96.2% specificity. As compared to standard HRM metrics produced using a maximized specificity on L-DNA-containing samples in this report, Youden-based metrics increased sensitivity by 66.67% but decreased specificity by 1.3%. Youden-based sample classification accuracy is illustrated in FIG. 15. In particular, standard HRM misclassification is illustrated by three S315T samples within the drug-susceptible cutoff range (FIG. 15).

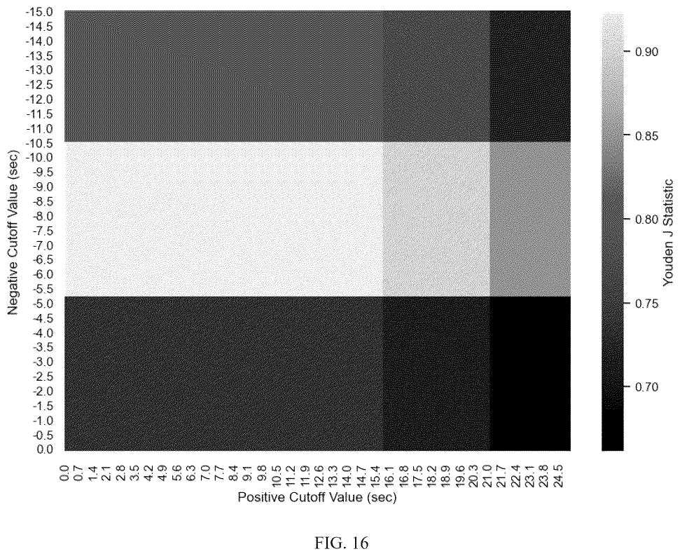

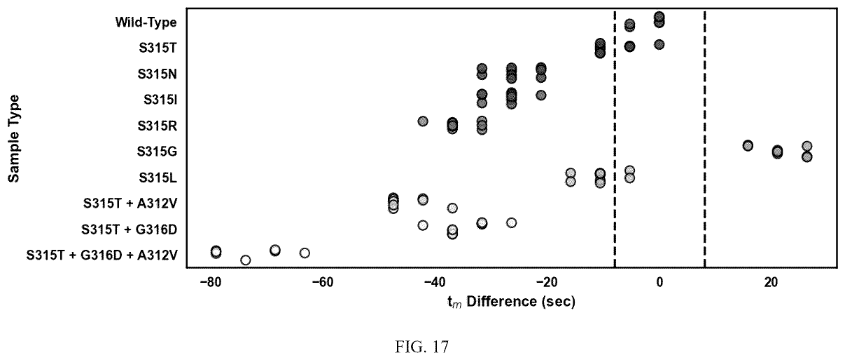

The Youden J Statistic was also utilized to determine the sensitivity and specificity of samples containing L-DNA and analyzed by LHRM analysis. The Youden J Statistic was calculated for upper and lower bound drug-susceptible cutoff values and plotted as a heatmap (FIG. 16). The maximized Youden J Statistic in the heatmap established tm difference cutoff values for LHRM sample classification as drug-susceptible or not. A sample was classified as drug-susceptible when sample tm difference was within the drug-susceptible tm difference cutoff range of −8 sec to 8 sec. Since true positives are known, LHRM was assessed for its sensitivity and specificity using this tm difference cutoff range when classifying drug-susceptibility among 9 true drug-susceptible samples (n=3 trials of wild-type in triplicate) and 79 true not drug-susceptible samples (n=3 trials of 9 variant types in triplicate, except variant S315T+G316D+A312V of one trial with a single replicate due to Cq exclusion). Using a maximized Youden J Statistic, LHRM performed at 100% sensitivity and 92.4% specificity. As compared to LHRM metrics produced using a maximized specificity on L-DNA-containing samples in this report, Youden-based metrics increased sensitivity by 22.2% but decreased specificity by 6.3%. Youden-based sample classification accuracy is illustrated in FIG. 17. In particular, LHRM misclassification is illustrated by six S315T samples within the drug-susceptible cutoff range (FIG. 17). This increase in false positive rate (i.e., misdiagnosis of not drug-susceptible cases) would be detrimental in the context of INH drug-susceptibility testing because those patients would remain on ineffective TB treatment by INH.

### L-DNA Overcomes the Effects of Sample-to-Sample Salt Variability on Classification

Instrument-based heating errors change the hybridization of a sample so this supplemental, proof-of-concept study tested whether other factors affecting hybridization could also be overcome using LHRM. While no other methodological errors were identified in HRM testing, it was speculated that some types of sample preparation errors could introduce systematic hybridization changes which could also be corrected using L-DNA. Possible errors may include culture media carryover, extraction errors, kit-to-kit master mix differences, or sample-to-sample salt concentration variability resulting from reagent pipetting errors. Of these, differences in salt concentration (which are known to alter DNA melting behavior and melting temperature) were the easiest to test. Using standard HRM, salt additions produced statistically significant changes in wild-type sample Tm as compared to standard preparation samples (p<0.05, Wilcoxon signed-rank test, 36 replicates per sample type) and would ultimately cause wild-type sample misclassification. Average PCR product Tm (mean±SD) was 82.49±0.10° C. for standard preparation wild-type samples and 83.18±0.10° C. for salt-additive wild-type samples. This suggests that preparation errors altering salt concentrations would not only likely misclassify wild-type samples but also likely misclassify variants by masking small mutation-induced melt changes.

Sample-to-sample preparation errors were further evaluated to determine if LHRM could overcome salt hybridization effects. LHRM reactions were first assessed using PCR product melt measurement alone for direct comparison to standard HRM reactions that did not contain L-DNA. Consistent with the previous results, standard melt analysis (using PCR product melt measurements alone) for LHRM reactions could not correctly characterize salt-additive wild-type samples as drug-susceptible. Specifically, there was a significant difference between salt-additive and standard preparation wild-type reactions when PCR product tm was used alone (p<0.05, Wilcoxon signed-rank test). Average PCR product tm (mean±SD) was 699.53±4.06 sec for standard preparation wild-type samples and 727.59±3.44 sec for salt-additive wild-type samples. However, as seen with heating variability, including a fixed amount of L-DNA in each sample provided a consistent comparator hybridization event that was used to reduce sample-to-sample sample preparation variability affecting hybridization. Using the L-DNA to PCR product melt difference, wild-type samples were correctly characterized as drug-susceptible whether or not they contained excess sodium. Specifically, wild-type tm differences were not significantly different when salt concentration increased (p>0.05, Wilcoxon signed-rank test, 36 replicates per sample type). Average tm difference (mean±SD) was 1.02±3.94 sec for standard preparation wild-type samples and 3.07±3.17 sec for salt-additive wild-type samples. As hypothesized, LHRM corrected for error-induced melt shifts by including L-DNA in every sample, and thus overcame hybridization effects of sample preparation and heating variability. This data was consistent with historical evidence that L-DNA has identical conformation transitions in the presence of salts as their D-DNA counterparts.

Experiments testing the effect of sample-to-sample salt concentration variability were performed in the Applied Biosystems™ QuantStudio™ 5 real-time PCR thermal cycler (Thermo Fisher Scientific #A28137). Reactions had a 20 μL final volume containing 1× of SensiFAST™ Probe No-ROX Kit (Bioline #BIO-86005), 1×LCGreen® Plus (BioFire® Defense, LLC #BCHM-ASY-005), and 250 nM of each katG-specific primer (MEP176 and MEP177). Each target sample contained a final concentration of wild-type (MEP183) single-stranded DNA target at 2×10⁶ copies per reaction. Salt-additive samples included 35 mM sodium chloride (Sigma Aldrich #S5150-1L) per reaction to simulate viral transport media equivalent salt contributions from possible extraction error. LHRM reactions included 2 μL of L-DNA mix with final copy counts of 1×10¹¹ copies TXR-labeled forward strand L-DNA (23FEB_katGf56_TXR) and 3×10¹¹ copies BHQ2-labeled reverse strand L-DNA (23FEB_katG_56_Rcmp+5_BHQ2) per reaction.

Salt-additive experiments assumed that thermal characteristics held constant plate-to-plate. Standard preparation and salt-additive wild-type samples were loaded into two plates of matching well-to-well standard preparation versus salt-additive samples. PCR reactions were initiated with a 95° C. hold for 2 min followed by 40 cycles of 95° C. for 5 sec and 59° C.¬ for 20 sec. Fluorescence was measured at the end of the annealing/extension step (59° C.). A high resolution melt was performed immediately following PCR by annealing 95° C. to 50° C. at 0.1° C./sec followed by melting 65° C. to 95° C. at 0.025° C./sec. Melt fluorescence was measured using the continuous acquisition mode. Double-stranded DNA PCR product fluorescence was monitored during PCR and during the melt reaction using LCGreen® Plus on the green optical channel (excitation 470±15/emission 520±15). Double-stranded L-DNA (in LHRM reactions) was monitored during the melt reaction using end-labeling (Texas Red fluorophore and Black Hole Quencher 2 quencher) on the orange fluorescence channel (excitation 580±10/emission 623±14).

PCR quantification cycle (Cq) was determined with the QuantStudio™ 5 Design and Analysis Software. Non-amplifying samples did not report Cq and were excluded from the data analysis. Amplifying samples with Cq over 35 were excluded from the data analysis because they did not achieve the PCR plateau phase. Tm was calculated with the proprietary QuantStudio™ 5 Design and Analysis Software based on the first derivative of fluorescence with respect to temperature. tm was calculated from the second degree Savitsky-Golay polynomials11 at each point (performed in MATLAB 2023A) based on the first derivative of fluorescence with respect to elapsed melt time. Salt concentration variability testing was evaluated using significant differences (Wilcoxon signed-rank test, significance level of α=0.95) well-to-well of standard preparation as compared to salt-additive wild-type (n=2 trials with 36 replicates in both standard HRM and LHRM). The Wilcoxon signed-rank test was performed for Tm in standard HRM and for tm and tm difference in LHRM. All statistics were performed in Microsoft® Excel 2022.

### Comparison of Equal Concentration 1:1 D-DNA and Unlabeled 1:1 L-DNA

Early development of LHRM assumed that unlabeled wild-type L-DNA and D-DNA of equivalent sequence, length, strand ratio, and strand concentration would produce the same melt temperature. This assumption only holds if an intercalating dye works in the same way for right-handed and left-handed enantiomeric DNA stereoisomers. In practice, unlabeled wild-type L-DNA and D-DNA (Table 11) of equivalent sequence, length, and concentration actually produced different melt temperatures across all three intercalating dyes tested (Table 10). D-DNA Tm was higher than L-DNA Tm across all three intercalating dyes (Table 10). The average L-DNA to D-DNA Tm difference varied by intercalating dye: 0.38° C. for EvaGreen®, 0.28° C. for EvaGreen® Plus, and 0.09° C. for LCGreen® Plus (Table 10). Although this data has not been previously reported in literature, the L-DNA versus D-DNA differences in melting behavior is speculated to be explained by intercalators fitting differently into left-handed versus right-handed enantiomeric DNA structures. Unique stereochemistry and shielded negative phosphate groups may alter intercalating dye mechanisms of action, thereby increasing L-DNA equivalent Tm. LCGreen® Plus was chosen as the intercalating dye of choice in LHRM because it presented the smallest Tm difference (0.09° C.) amongst the dyes tested.

These studies were performed using wild-type katG sequence strands 56-bases in length. L-DNA was unlabeled to assess a true L-DNA versus D-DNA comparison of melting across three intercalators: EvaGreen®, EvaGreen® Plus, and LCGreen® Plus. Triplicates were prepared for each intercalating dye containing either 1:1 L-DNA or 1:1 D-DNA. Reactions were loaded into the 96-well plate such that each set of triplicates were in the same column, L-DNA samples were in the same rows as their D-DNA counterparts, and each set of intercalating dye samples were in mirrored locations across the left and right halves of the plate. Reactions had a 20 μL final volume containing 1× of SensiFAST™ Probe No-ROX Kit (Bioline #BIO-86005) and 1× of intercalating dye using either EvaGreen® Dye (Biotium #31000), EvaGreen® Plus Dye (Biotium #31077, or LCGreen® Plus (BioFire® Defense, LLC #BCHM-ASY-005). Each L-DNA reaction included 2 μL of L-DNA mix with final copy counts of 7.525×10¹¹ copies unlabeled forward strand L-DNA (23FEB_katGf56) and 7.525×10¹¹ copies unlabeled reverse strand L-DNA (23FEB_katGRcmp56) per reaction (Table 11). Each D-DNA reaction included 2 μL of D-DNA mix for final copy counts of 7.525×10¹¹ copies forward strand D-DNA (MEP213) and 7.525×10¹¹ copies reverse strand D-DNA (MEP214) per reaction (Table 11). A high-resolution melt was performed by annealing 95° C. to 50° C. at 0.1° C./sec followed by melting 65° C. to 95° C. at 0.1° C./sec. Melt fluorescence was measured using the continuous acquisition mode and monitored using intercalating dye on the green optical channel (excitation 470±15/emission 520±15). Tm was calculated with the proprietary QuantStudio™ 5 Design and Analysis Software based on the first derivative of fluorescence with respect to temperature. Tm difference was calculated as L-DNA Tm subtracted from D-DNA Tm in the well location mirrored across the 96-well plate.

### CONCLUSION

By including L-DNA for reagent-based calibration in every sample, LHRM successfully classifies PCR melt products as INH-susceptible or not based on within-sample differences between an L-DNA comparator and an unknown PCR product. LHRM achieves comparable classification specificity and sensitivity to standard HRM with single sample analysis. With further development, LHRM shows promise as an initial drug susceptibility screen that may be incorporated into the TB clinical treatment algorithm where real-time PCR instruments are available.

### Example 2. Single-Sample Melt-Based Screening for Rifampicin Susceptibility in the Emerging Mutation Hotspot at rpoB Codon 491

Based on sequencing data, mutations at rpoB codon 491 of Mycobacterium tuberculosis are associated with rifampicin resistance but current commercial and WHO-endorsed genotypic tests fail to detect them. As a result, resistant infections go untreated, driving transmission and multidrug resistance. A real-time PCR assay by André et al. specifically screens for 1491F but omits other codon 491 mutations. To address this gap, a single-sample screening method using asymmetric PCR followed by melt analysis was developed for the three sequence-identified variants, I491F/N/M. Each sample contained a melt probe matching the susceptible sequence, which, after asymmetric PCR spanning codon 491, hybridized with the excess strand to form a duplex. The duplex's melt temperature (Tm) was then measured. To enable single-sample classification, each reaction also included double-stranded L-DNA identical to the probe and wild-type PCR product duplex. Susceptibility was determined by the within-sample Tm difference between the probe-product and L-DNA duplexes. The approach was evaluated and compared to the André assay across two calibrated PCR instruments using synthetic rpoB wild-type and variant sequences. As expected, the André assay distinguished wild-type from I491F samples but misclassified I491N and I491M samples based on multi-sample Tm comparison. In contrast, our single-sample classification strategy used within-sample Tm differences, classifying samples as rifampicin-susceptible when the within-sample Tm difference was less than 0.83° C. Using this approach, the method achieved 100% sensitivity and 100% specificity across both PCR instruments. Although demonstrated for rpoB codon 491, this assay design is readily adaptable to any other sequence-identified, clinically significant mutation hotspot.

### INTRODUCTION

The global burden of tuberculosis (TB) is exacerbated by the emergence of drug-resistant strains with mutations that render drug treatments ineffective. In particular, mutations associated with rifampicin resistance pose a significant challenge to TB control by compromising the efficacy of rifampicin, the most potent first-line anti-TB drug. Each year, approximately half a million people are infected with rifampicin-resistant TB, creating a significant barrier to disease management and control. Rapid and accurate drug susceptibility testing is critical to ensuring that patients receive the most effective treatment regimens and reduce the spread of drug-resistant TB strains.

Sequencing data suggests that the vast majority (˜96%) of rifampicin resistance is associated with mutations in the rifampicin resistance determinant region (RRDR), an 81-base-pair section of the rpoB gene spanning Mycobacterium tuberculosis (MTB) rpoB codons 426 to 452. As a result, current commercial and WHO-endorsed genotypic drug susceptibility tests, such as line-probe assays and Xpert MTB/RIF, focus on detecting rifampicin resistance-related mutations within the RRDR6.

However, the landscape of rifampicin resistance continues to evolve, with new resistance-related mutations emerging in rpoB hotspots outside the current RRDR screening region. Mutations at rpoB codon 491, for example, confer rifampicin resistance but remain undetected by current WHO-endorsed genotypic susceptibility tests and are routinely misidentified as rifampicin susceptible by phenotypic drug susceptibility tests. The rpoB I491F mutation, first reported in Eswatini in 2015—where it had been silently transmitted since 2009 and caused a multidrug-resistant outbreak—illustrates this screening gap. While I491F accounts for approximately 0.5% of global rifampicin resistance, it is far more prevalent in certain regions, accounting for over 50% of rifampicin-resistant cases in Eswatini. Despite its clinical significance, no current commercial or WHO-endorsed tests screen for rifampicin susceptibility at codon 491. Moreover, the I491F mutation continues to spread, as indicated by its recent detection in new geographical regions. In addition, newly identified codon 491 variants, such as I491N and I491M, have also been linked to rifampicin resistance. Susceptibility screening diagnostics are not keeping pace with these evolving resistance-related mutations.

While sequencing has been used for epidemiological discovery and study of I491 mutations, the published literature describes only one real-time PCR assay developed for I491F-specific screening. In this work by Andre et al., the assay was designed to screen for the I491F mutation by specifically targeting single nucleotide variants (SNVs) at the first nucleotide base of codon 491, rpoB nucleotide 1471. Although the André assay's selective mechanism of action successfully classifies the I491F variant as not susceptible, its specificity prevents its use as is to screen for other resistance-related mutations emerging in the rpoB codon 491 hotspot.

In the present disclosure, a single-sample screening strategy using asymmetric PCR followed by melt analysis is developed to address the unmet need for comprehensive rifampicin susceptibility screening across all three nucleotides of the rpoB codon 491 mutation hotspot. The generalizable method as described herein, which is herein alternatively referred to as the term “Single-Sample Melt Analysis for Screening Hotspots (SMASH)”, is applied as a proof of concept for screening the rpoB codon 491 hotspot. The method as described herein screens for SNVs within rpoB nucleotides 1447-1476 using two internal comparators for rifampicin susceptibility in every sample: a single-stranded melt probe and a double-stranded left-helical (L)-DNA. Single-sample classification is achieved by measuring melt temperature (Tm) shifts between two duplexes: the susceptible melt probe hybridized to the asymmetric PCR excess strand and a double-stranded L-DNA with the same Tm as the susceptible probe-wild-type product duplex. To validate SMASH for I491 screening, we tested its performance across two calibrated real-time PCR instruments (QuantStudio™ 5 and Rotor-Gene® Q) and compared it to the André I491F assay. This evaluation used samples containing synthetic rpoB wild-type and three sequence-identified rpoB codon 491 variants (I491F/N/M) to validate the assay for SNVs at all three nucleotides (1471-1473) of the rpoB codon 491 hotspot (Table 12). The SMASH method strategy provides a generalizable framework for developing single-sample melt-based assays to screen for clinically significant mutations identified through sequencing.

TABLE 12
Sequences (written 5′-to-3′) of the drug-susceptible
wild-type rpoB and three sequence-identified rpoB codon
491 variants (I491F/N/M). Each variant has rifampicin-
resistance-related mutations indicated as underlined.

| Variant | Codon 491 Sequence |
|---|---|
| Wild-type | ATC |
| I491F | TTC |
| I491N | AAC |
| I491M | ATA |

### Materials and Methods

Single-Sample Melt Analysis for Screening Hotspots (SMASH) Applied to the Hotspot at rpoB Codon 491

The single-sample melt analysis test was developed using the sequence of the drug-susceptible TB rpoB gene. A single primer set was designed to cover rifampicin resistance-related mutations at the rpoB codon 491 emerging hotspot and its neighboring bases. SMASH uses asymmetric PCR followed by melt analysis, where asymmetric PCR generates both single-stranded asymmetric products and full-length amplicons.

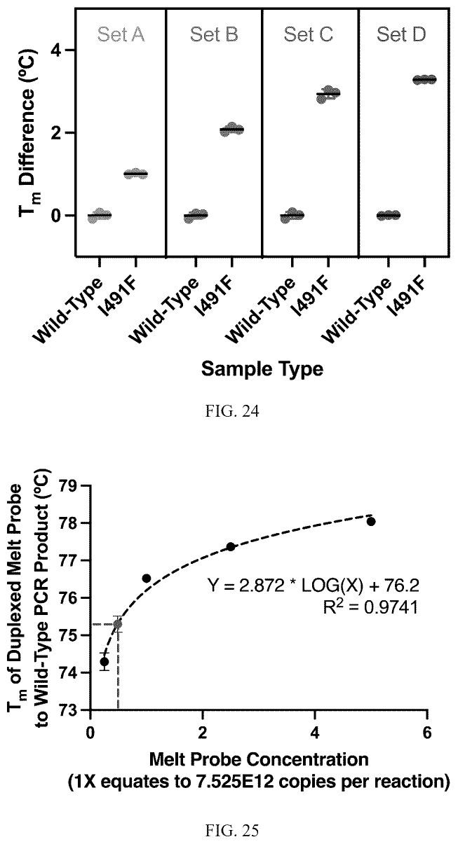

A drug-susceptible melt probe was synthesized as a reverse complement sequence to the known drug-susceptible rpoB sequence. The 30-base melt probe sequence was identical to the corresponding region of the excess asymmetric PCR product strand between the primers. The susceptible melt probe had a 3′ C3 spacer to prevent extension during PCR. The 30-base susceptible melt probe formed a duplex with the 66-base asymmetric PCR product, regardless of whether the PCR product had a susceptible sequence. If a SNV was present in the PCR product strand, the probe-product duplex had a nucleotide mismatch that destabilized the duplex and caused a melt mismatch with the susceptible L-DNA's Tm. Strategic selection of the melt probe sequence is detailed herein (FIG. 24).

Similar to prior L-DNA-based screening for isoniazid susceptibility, this assay added double-stranded L-DNA to every sample to serve as a standard melt comparator for rifampicin susceptibility. The L-DNA drug-susceptible comparator was synthesized using left-helical enantiomeric DNA bases (i.e., L-DNA) with an identical sequence to the known drug-susceptible rpoB sequence. The 30-base L-DNA forward strand was synthesized with the same length and sequence as the drug-susceptible melt probe. The forward L-DNA strand had a 5′ Texas Red end-label. The reverse complement L-DNA strand was unlabeled.

The assay was performed on a test system of synthetic rpoB wild-type and three variant sample types. Single-stranded PCR targets were synthesized with D-DNA sequences of drug-susceptible wild-type rpoB (H37Rv: Rv0667: 759807-763325) and three clinically relevant drug-resistant rpoB mutants (Table 12). Each of the three selected mutants included SNVs at one of the three nucleotides of rpoB codon 491, offering robust SMASH assay validation for rifampicin susceptibility at rpoB codon 491. Detailed information on the DNA oligonucleotide sequences used in these studies is shown in Table 13. All DNA oligonucleotides employed for development and testing were synthesized by Integrated DNA technologies (Coralville, Iowa, USA) or biomers.net (Ulm, Baden-Württemberg, Germany).

The SMASH assay was performed using two real-time PCR thermal cyclers, the QuantStudio™ 5 instrument (Thermo Fisher Scientific #A28137) with a 96-well plate sample setup and the Rotor-Gene® Q 5plex Platform (Qiagen #9001570) with a spinning rotor sample setup. Assay performance was compared across two real-time PCR platforms to demonstrate its versatility and adaptability across systems, ensuring broader TB screening access using existing instruments rather than requiring assay-specific equipment investments. All reactions had a 20 μL final volume containing 1× of SensiFAST™ Probe No-ROX Kit (Bioline #BIO-86005), 1×SYBR® Green I (Sigma-Aldrich® #S9430), 0.25 μM rpoB-specific forward primer (SMASH_FWDpri), 0.025 μM rpoB-specific reverse primer (SMASH_REVpri), 3.7625×10¹² copies of C3-blocked susceptible melt probe (SMASH_SusMeltProbeC3), 4×10¹¹ copies of Texas Red-labeled forward strand L-DNA (SMASH_FWDsusLDNAtxr), and 4×10¹¹ copies of unlabeled reverse strand L-DNA (SMASH_REVCOMPsusLDNA). Each target sample contained a final concentration of wild-type (TARGETrpoB_WT) or mutant (TARGETrpoB_I491F, TARGETrpoB_I491N, TARGETrpoB_I491M) single-stranded DNA target at 2×10⁶ copies per reaction. In preliminary tests, the susceptible melt probe concentration was reduced per reaction to align the Tm of the duplexed susceptible melt probe with the wild-type asymmetric PCR product to that of the double-stranded L-DNA (FIG. 25). This concentration-based strategy, similarly employed in prior L-DNA-based work, enabled single-sample testing by ensuring that the susceptible L-DNA and the probe-product duplex had nearly identical melt characteristics when the sequences matched (wild-type rpoB) but differed if there was a sequence mismatch (rpoB I491 variants).

QuantStudio™ 5 PCR reactions were initiated with a 95° C. hold for 2 min followed by 40 cycles of 95° C. for 5 sec and 57° C. for 20 sec. A high resolution melt was performed immediately following PCR by annealing 95° C. to 50° C. at 0.1° C./sec followed by melting 65° C. to 90° C. at 0.025° C./sec (continuous acquisition mode). Double-stranded DNA PCR product fluorescence was measured during PCR and during melting using SYBR® Green I on the green optical channel (excitation 470±15/emission 520±15). Double-stranded L-DNA fluorescence was monitored on the orange optical channel (emission 623±14) during the melt reaction using iFRET33 with SYBR® Green I as the FRET donor and Texas Red on the L-DNA forward strand as the FRET acceptor.

Rotor-Gene® Q PCR reactions followed identical reaction setup and PCR cycling conditions to the QuantStudio™ 5. Melt analysis data acquisition rates and optical channel specifications varied between instruments due to inherent differences in their design. The Rotor-Gene® Q's high resolution melt was performed immediately following PCR by annealing 95° C. to 50° C. at 0.1° C./step followed by melting 65° C. to 95° C. at 1° C./step with 5 sec hold per step. The Rotor-Gene Q's SYBR® Green I optical channel was specified by excitation 470±10/emission 510±5 while the iFRET acceptor orange optical channel was specified by excitation 470±10/emission 610±5.

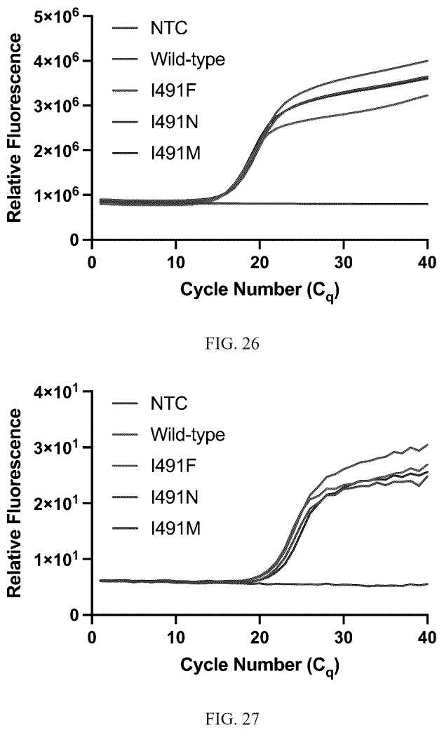

PCR quantification cycle (Cq) was determined with the QuantStudio™ 5 Design and Analysis Software and the Rotor-Gene® Q Series Software. Representative PCR amplification curves of samples for the QuantStudio™ 5 and Rotor-Gene® Q are included in FIG. 26 and FIG. 27, respectively. Tm was calculated with the proprietary QuantStudio™ 5 Design and Analysis Software based on the first derivative of fluorescence with respect to temperature. Within-sample melt difference and statistical significance were performed independently for the QuantStudio™ 5 and Rotor-Gene® Q data sets. A within-sample melt difference was calculated between the Tm of each sample's probe-product duplex and the Tm of each sample's L-DNA. Significance was evaluated using within-sample melt difference comparison (one-way ANOVA, Tukey's post hoc test for multiple comparisons, significance level of α=0.95) across I491F, I491N, and I491F as compared to wild-type (n=6 trials in triplicate per sample type per instrument). Since true positives were known, the SMASH assay was assessed for its sensitivity and specificity using ROC analysis of within-sample Tm difference metrics (Wilson/Brown method, Table 15). Across both the QuantStudio™ 5 and Rotor-Gene® Q sample sets, a Tm difference cutoff point was selected by prioritizing maximized specificity (to decrease the false positive rate, i.e., decrease the misdiagnosis of variant samples as drug-susceptible), followed by maximized sensitivity, when classifying each test sample as drug-susceptible or not. All samples were classified as rifampicin-susceptible using a within-sample Tm difference less than 0.83° C. for the QuantStudio™ 5 and Rotor-Gene® Q sample sets (n=6 trials in triplicate per sample type per instrument). All statistics were performed in GraphPad Prism version 10.0.

### André 1491F Assay

The Andre et al. assay for I491F-specific detection14 was replicated as closely as possible to the original published method to compare the performance of the developed SMASH assay with existing real-time PCR I491F screening capabilities. The assay's multiplex allele-specific PCR strategy used a combination of three primers to amplify allele-specific fragments distinguished by their size. According to prior work, rifampicin-susceptible wild-type samples were expected to form 301 base-pair PCR amplicons while I491F samples were expected to form 70 base-pair PCR amplicons. The André I491F assay was performed using the same synthetic targets (Table 12) and QuantStudio™ 5 instrument as the SMASH assay for comparison. All reactions had a 25 μL final volume containing 1× of SensiFAST™ Probe No-ROX Kit, 1×SYBR® Green I, and 0.5 μM of each rpoB-specific MAS-PCR primer (Andre_FWDpri_outer, Andre_FWDpri_overlapping1471, Andre_REVpri). Each target sample contained a final concentration of wild-type (TARGETrpoB_WT) or mutant (TARGETrpoB_I491F, TARGETrpoB_I491N, TARGETrpoB_I491M) single-stranded DNA target at 5×10⁶ copies per reaction. Detailed information on the DNA oligonucleotide sequences used in these studies is shown in Table 13. PCR reactions were initiated with a 95° C. hold for 2 min followed by 35 cycles of 95° C. for 5 sec and 56° C. for 20 sec. A high resolution melt was performed immediately following PCR by melting 65° C. to 97° C. at 10 readings per° C. (dissociation acquisition mode). Double-stranded DNA PCR product fluorescence was measured during PCR and during melting using SYBR® Green I on the green optical channel (excitation 470±15/emission 520±15). Protocol deviations from the prior work 14 included type of real-time PCR instrument, type of PCR master mix, melt cycling conditions, use of synthetic targets instead of clinical samples, and expansion to include variants I491N and I491M in addition to I491F.

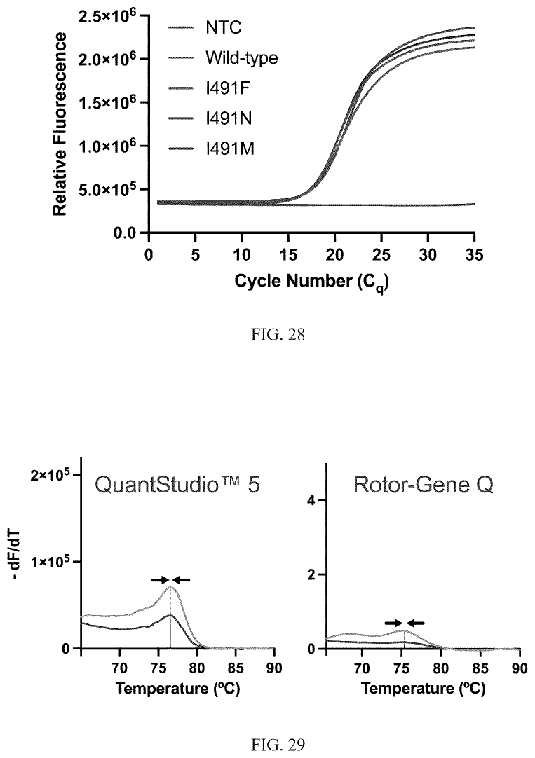

QuantStudio™ 5 Design and Analysis Software determined PCR Cq and Tm (based on the first derivative of fluorescence with respect to temperature). Representative PCR amplification curves of samples are included in FIG. 28. Although André et al. did not explicitly state how to determine melt difference criteria14, the present study calculated melt difference, or Tm difference, between the Tm of each sample's PCR product and the Tm of the global average wild-type melt temperature from the sample set (n=3 trials in triplicate per sample type). Significance was evaluated using melt difference comparison (one-way ANOVA, Tukey's post hoc test for multiple comparisons, significance level of α=0.95) across I491F, I491N, and I491F as compared to wild-type (n=3 trials in triplicate per sample type). Per André et al. 14, samples were classified as rifampicin-susceptible using a Tm difference less than 5.24° C. (n=3 trials in triplicate per sample type). All statistics were performed in GraphPad Prism version 10.0.

### Oligonucleotides

TABLE 13
Oligonucleotide sequences designed for the SMASH assay applied to rpoB codon 491
and the Andre I491F assay.

| | | | NA | |
|---|---|---|---|---|
| | DNA | | Base | |
| ID | Type | Description | Change | Sequence (5′ → 3′) |
| SMASH_F | D- | SMASH assay rpoB | N/A | (SEQ ID NO: 30) |
| WDpri | DNA | forward primer (66 base- | | GATGTGCCCGATCGAAACC |
| | | pair amplicon) | | |
| SMASH_ | D- | SMASH assay rpoB | N/A | (SEQ ID NO: 31) |
| REVpri | DNA | reverse primer (66 base- | | GCGTACACCGACAGCGA |
| | | pair amplicon) | | |
| SMASH_S | D- | SMASH assay | N/A | (SEQ ID NO: 32) |
| usMeltPro | DNA | rifampicin-susceptible | | GCCGATCAGACCGATGTTGGGCC |
| beC3 | | melt probe with 3′ C3 | | CCTCAGG/3SpC3/ |
| | | spacer to block PCR | | |
| | | extension | | |
| SMASH_F | L- | SMASH assay | N/A | (SEQ ID NO: 33) |
| WDsusLD | DNA | rifampicin-susceptible | | Texas Red/ |
| NAtxr | | L-DNA comparator | | GCCGATCAGACCGATGTTGGGCC |
| | | forward strand with 5′ | | CCTCAGG |
| | | Texas Red-X | | |
| SMASH_ | L- | SMASH assay | N/A | (SEQ ID NO: 34) |
| REVCOM | DNA | rifampicin-susceptible | | CCTGAGGGGCCCAACATCGGTCT |
| PsusLDNA | | L-DNA comparator | | GATCGGC |
| | | reverse complement | | |
| | | strand with no | | |
| | | modification | | |
| Andre_F | D- | André 1491F assay | N/A | (SEQ ID NO: 35) |
| WDpri_ | DNA | forward primer (301 | | TGGAGTACGTGCCCTCGTC |
| outer | | base-pair amplicon) | | |
| Andre_F | D- | André I491F assay | N/A | (SEQ ID NO: 36) |
| WDpri_ov | DNA | forward primer including | | GGCCCAACATCGGTCTGA |
| erlapping1 | | nucleotide 1471 of rpoB | | |
| 471 | | codon 471 (70 base-pair | | |
| | | amplicon) | | |
| Andre_RE | D- | André assay reverse | N/A | (SEQ ID NO: 37) |
| Vpri | DNA | primer (301 or 70 base- | | GTGGCCACCGACACCATCT |
| | | pair amplicons) | | |
| TARGETr | D- | Wild-type rifampicin- | N/A | (SEQ ID NO: 38) |
| poB_WT | DNA | susceptible target | | GATGTGCCCGATCGAAACCCCTG |
| | | | | AGGGGCCCAACATCGGTCTGATC |
| | | | | GGCTCGCTGTCGGTGTACGCGCG |
| | | | | GGTCAACCCGTTCGGGTTCATCG |
| | | | | AAACGCCGTACCGCAAGGTGGTC |
| | | | | GACGGCGTGGTTAGCGACGAGAT |
| | | | | CGTGTACCTGACCGCCGACGAGG |
| | | | | AGGACCGCCACGTGGTGGCACA |
| | | | | GGCCAATTCGCCGATCGATGCGG |
| | | | | ACGGTCGCTTCGTCGAGCCGCGC |
| | | | | GTGCTGGTCCGCCGCAAGGCGGG |
| | | | | CGAGGTGGAGTACGTGCCCTCGT |
| | | | | CTGAGGTGGACTACATGGACGTC |
| | | | | TCGCCCCGCCAGATGGTGTCGGT |
| | | | | GGCCAC |
| TARGETr | D- | I491F mutant target | A1471T | (SEQ ID NO: 39) |
| poB_I491 | DNA | | | GATGTGCCCGATCGAAACCCCTG |
| F | | | | 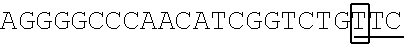 |
| | | | | GGCTCGCTGTCGGTGTACGCGCG |
| | | | | GGTCAACCCGTTCGGGTTCATCG |
| | | | | AAACGCCGTACCGCAAGGTGGTC |
| | | | | GACGGCGTGGTTAGCGACGAGAT |
| | | | | CGTGTACCTGACCGCCGACGAGG |
| | | | | AGGACCGCCACGTGGTGGCACA |
| | | | | GGCCAATTCGCCGATCGATGCGG |
| | | | | ACGGTCGCTTCGTCGAGCCGCGC |
| | | | | GTGCTGGTCCGCCGCAAGGCGGG |
| | | | | CGAGGTGGAGTACGTGCCCTCGT |
| | | | | CTGAGGTGGACTACATGGACGTC |
| | | | | TCGCCCCGCCAGATGGTGTCGGT |
| | | | | GGCCAC |
| TARGETr | D- | I491N mutant target | T1472A | (SEQ ID NO: 40) |
| poB_I491 | DNA | | | GATGTGCCCGATCGAAACCCCTG |
| N | | | | 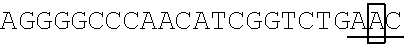 |
| | | | | GGCTCGCTGTCGGTGTACGCGCG |
| | | | | GGTCAACCCGTTCGGGTTCATCG |
| | | | | AAACGCCGTACCGCAAGGTGGTC |
| | | | | GACGGCGTGGTTAGCGACGAGAT |
| | | | | CGTGTACCTGACCGCCGACGAGG |
| | | | | AGGACCGCCACGTGGTGGCACA |
| | | | | GGCCAATTCGCCGATCGATGCGG |
| | | | | ACGGTCGCTTCGTCGAGCCGCGC |
| | | | | GTGCTGGTCCGCCGCAAGGCGGG |
| | | | | CGAGGTGGAGTACGTGCCCTCGT |
| | | | | CTGAGGTGGACTACATGGACGTC |
| | | | | TCGCCCCGCCAGATGGTGTCGGT |
| | | | | GGCCAC |
| TARGETr | D- | I491M mutant target | C1473 | (SEQ ID NO: 41) |
| poB_I491 | DNA | | A | GATGTGCCCGATCGAAACCCCTG |
| M | | | | 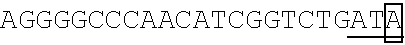 |
| | | | | GGCTCGCTGTCGGTGTACGCGCG |
| | | | | GGTCAACCCGTTCGGGTTCATCG |
| | | | | AAACGCCGTACCGCAAGGTGGTC |
| | | | | GACGGCGTGGTTAGCGACGAGAT |
| | | | | CGTGTACCTGACCGCCGACGAGG |
| | | | | AGGACCGCCACGTGGTGGCACA |
| | | | | GGCCAATTCGCCGATCGATGCGG |
| | | | | ACGGTCGCTTCGTCGAGCCGCGC |
| | | | | GTGCTGGTCCGCCGCAAGGCGGG |
| | | | | CGAGGTGGAGTACGTGCCCTCGT |
| | | | | CTGAGGTGGACTACATGGACGTC |
| | | | | TCGCCCCGCCAGATGGTGTCGGT |
| | | | | GGCCAC |

DNA is denoted as D-DNA or L-DNA.
Nucleotides in rpoB codon 491 are underlined.

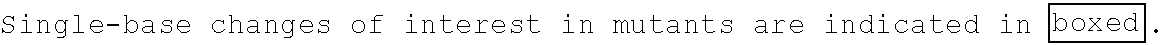

### Selecting Susceptible Melt Probe Sequence

A key component of the SMASH assay was the drug-susceptible melt probe, designed as the reverse complement of the drug-susceptible rpoB sequence. The probe sequence was selected to maximize the melt temperature (Tm) difference between its duplex with a wild-type rpoB strand and its duplex with an I491F variant strand (FIG. 24).

To optimize this melt difference, we systematically varied the probe's 5′ end while keeping its 3′ end constant (Table 14). Four probe sets (A-D) were designed, progressively increasing the offset between the I491F mutation (nucleotide 1471) and the probe's 5′ end. This offset grew one at a time from two bases in Set A (27 bases long) to five bases in Set D (30 bases long, Table 14). Prior studies suggest that increasing the mismatch offset destabilizes the duplex more significantly, leading to greater melt shifts.

For preliminary characterization, we used unblocked melt probe mimics. Melt analysis was performed by comparing the Tm differences between the susceptible melt probe mimic (reverse complement to wild-type rpoB) hybridized with either wild-type rpoB or I491F asymmetric PCR excess product strand mimics (rpoB as is). Each probe set (A-D) was tested with corresponding product strands of increasing length and offset (Table 14).

Consistent with duplex destabilization principles, increasing the mismatch offset led to larger melt shifts between the probe—I491F duplex and the probe—wild-type duplex (FIG. 24). Tm differences were calculated between each sample's duplex Tm and the average Tm of duplexed melt probe to wild-type for that particular set. All four sets had average Tm differences of 0° C. for susceptible melt probe bound to wild-type rpoB. Average Tm difference for susceptible melt probe bound to I491F increased progressively across sets: 1.01° C. for Set A (27-bases long, 2-base offset), 2.08° C. for Set B (28-bases long, 3-base offset), 2.94° C. for Set C (29-bases long, 4-base offset), and 3.28° C. for Set D (30-bases long, 5-base offset). Since Set D exhibited the largest Tm difference for I491F discrimination, its probe sequence (SMASH_SetD_mimicSusMeltProbe) was selected for final assay development. This optimized probe (C3-blocked susceptible melt probe, SMASH SusMeltProbeC3; Table 13) facilitated clear discrimination between I491F samples and susceptible melt profiles (FIG. 21A and FIG. 22A). Similarly, distinct melt differences were observed for I491N and I491M mismatches (FIG. 21A and FIG. 22A).

Methods: The melt probe characterization studies were performed using the QuantStudio™ 5 real-time PCR instrument (Thermo Fisher Scientific #A28137). Reactions had a 20 μL final volume containing 1× of SensiFAST™ Probe No-ROX Kit (Bioline #BIO-86005), 1×LCGreen® Plus (BioFire® Defense, LLC #BCHM-ASY-005), 7.525×10¹² copies of susceptible melt probe (wild-type reverse complement), and 7.525×10¹² copies of single-stranded target (either rpoB wild-type or I491F mutant). Set A reactions included melt probe SMASH SetA mimicSusMeltProbe bound to wild-type target SMASH SetA_mimicAsymPCRprod_WT or I491F target SMASH_SetA_mimicAsymPCRprod_I491F. Set A oligonucleotides were 27-bases in length and had a 2-base offset of rpoB nucleotide 1471. Set B reactions included melt probe SMASH SetB_mimicSusMeltProbe bound to wild-type target SMASH_SetB_mimicAsymPCRprod_WT or I491F target SMASH_SetB_mimicAsymPCRprod_I491F. Set B oligonucleotides were 28-bases in length and had a 3-base offset of rpoB nucleotide 1471. Set C reactions included melt probe SMASH SetC_mimicSusMeltProbe bound to wild-type target or I491F target SMASH_SetC_mimicAsymPCRprod_WT SMASH_SetC_mimicAsymPCRprod_I491F. Set C oligonucleotides were 29-bases in length and had a 4-base offset of rpoB nucleotide 1471. Set D reactions included melt probe SMASH SetD_mimicSusMeltProbe bound to wild-type target SMASH_SetD_mimicAsymPCRprod_WT or I491F target SMASH SetD_mimicAsymPCRprod_I491F. Set D oligonucleotides were 30-bases in length and had a 5-base offset of rpoB nucleotide 1471.

The high resolution melt was initiated with a 95° C. hold for 2 min followed by annealing 95° C. to 50° C. at 0.1° C./sec followed by melting 60° C. to 95° C. at 0.025° C./sec (continuous acquisition mode). Double-stranded DNA fluorescence was monitored during the melt reaction using LCGreen® Plus on the green optical channel (excitation 470±15/emission 520±15).

TABLE 14
Oligonucleotide sequences designed for preliminary susceptible melt probe sequence
design in the SMASH assay.

| | | Offset of Nucleotide | |
|---|---|---|---|
| | | 1471 | |
| | | (from 3′ End of | |
| | | Asymmetric PCR | |
| | | Product Mimics and | |
| | | from 5′ End of Melt | |
| ID | Description | Probe Mimics) | Sequence (5′ → 3′) |
| SMASH_SetA_mimics | Set A susceptible melt | 2 | (SEQ ID NO: 42) |
| usMeltProbe | probe mimic as reverse | | GATCAGACCGATGTT |
| | complement of wild-type | | GGGCCCCTCAGG |
| | rpoB (27 bases in length) | | |
| SMASH_SetA_mimicA | Set A asymmetric PCR | 2 | (SEQ ID NO: 43) |
| symPCRprod_WT | product mimic as wild- | | CCTGAGGGGCCCAA |
| | type rpoB (27 bases in | | CATCGGTCTGATC |
| | length) | | |
| SMASH_SetA_mimicA | Set A asymmetric PCR | 2 | (SEQ ID NO: 44) |
| symPCRprod_I491F | product mimic as rpoB | | CCTGAGGGGCCCAA |
| | I491F variant (27 bases in length) | | 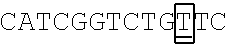 |
| SMASH_SetB_mimicS | Set B susceptible melt | 3 | (SEQ ID NO: 45) |
| usMeltProbe | probe mimic as reverse | | CGATCAGACCGATGT |
| | complement of wild-type | | TGGGCCCCTCAGG |
| | rpoB (28 bases in length) | | |
| SMASH_SetB_mimicA | Set B asymmetric PCR | 3 | (SEQ ID NO: 46) |
| symPCRprod_WT | product mimic as wild- | | CCTGAGGGGCCCAA |
| | type rpoB (28 bases in | | CATCGGTCTGATCG |
| | length) | | |
| SMASH_SetB_mimicA | Set B asymmetric PCR | 3 | (SEQ ID NO: 47) |
| symPCRprod_I491F | product mimic as rpoB | | CCTGAGGGGCCCAA |
| | I491F variant (28 bases in length) | | 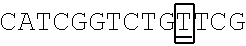 |
| SMASH_SetC_mimicS | Set C susceptible melt | 4 | (SEQ ID NO: 48) |
| usMeltProbe | probe mimic as reverse | | CCGATCAGACCGATG |
| | complement of wild-type | | TTGGGCCCCTCAGG |
| | rpoB (29 bases in length) | | |
| SMASH_SetC_mimicA | Set C asymmetric PCR | 4 | (SEQ ID NO: 49) |
| symPCRprod_WT | product mimic as wild- | | CCTGAGGGGCCCAA |
| | type rpoB (29 bases in | | CATCGGTCTGATCGG |
| | length) | | |
| SMASH_SetC_mimicA | Set C asymmetric PCR | 4 | (SEQ ID NO: 50) |
| symPCRprod_I491F | product mimic as rpoB | | CCTGAGGGGCCCAA |
| | I491F variant (29 bases in length) | | 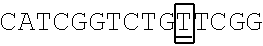 |
| SMASH_SetD_mimicS | Set D susceptible melt | 5 | (SEQ ID NO: 51) |
| usMeltProbe | probe mimic as reverse | | GCCGATCAGACCGAT |
| | complement of wild-type | | GTTGGGCCCCTCAG |
| | rpoB (30 bases in length) | | G |
| SMASH SetD_mimicA | Set D asymmetric PCR | 5 | (SEQ ID NO: 52) |
| symPCRprod_WT | product mimic as wild- | | CCTGAGGGGCCCAA |
| | type rpoB (30 bases in | | CATCGGTCTGATCGG |
| | length) | | C |
| SMASH SetD_mimicA | Set D asymmetric PCR | 5 | (SEQ ID NO: 53) |
| symPCRprod_I491F | product mimic as rpoB | | CCTGAGGGGCCCAA |
| | I491F variant (30 bases in length) | | 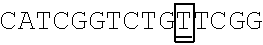 |
| | | | C |

All sequences are synthesized with D-DNA.
Underlining indicates rpoB nucleotide 1471 (the SNV location for 1491F).

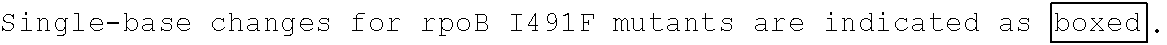

### Tuning Susceptible Melt Probe Concentration

Classifying susceptibility with the SMASH assay relied on a within-sample melt difference between susceptible double-stranded L-DNA and duplexed susceptible melt probe to wild-type asymmetric PCR product. The L-DNA and probe-product duplex had nearly identical melt characteristics when the sequences matched (wild-type rpoB, top left panels in FIG. 21B and FIG. 22B) but differed if there was a sequence mismatch (rpoB I491 variants, top right and lower two panels in FIG. 21B and FIG. 22B). Although not critical, melt matching made susceptibility classification visually apparent from the melt curve. To ensure matching melt characteristics between susceptible L-DNA and duplexed susceptible melt probe to wild-type asymmetric PCR product, preliminary experiments were performed varying melt probe strand concentration. Previous reports have established that total DNA concentration and strand ratio affect the melt temperature. Prior L-DNA-based work has also employed strand concentration tuning strategies to achieve melt matching for a susceptible L-DNA melt comparator. In the present study, the susceptible melt probe concentration per reaction was reduced such that the Tm of the duplexed susceptible melt probe to wild-type asymmetric PCR product matched the average Tm of the double-stranded L-DNA.

As melt probe concentration per reaction was lowered, probe-wild-type product duplex Tm decreased (FIG. 25). This phenomenon was used to achieve an empirical melt match between drug-susceptible L-DNA and the duplexed probe to wild-type asymmetric PCR product (top left panels in FIG. 21B and FIG. 22B). A positive logarithmic relationship between the number of susceptible melt probe copies per reaction and probe-wild-type duplex Tm indicated that decreasing probe concentration down to 0.487×, or 3.665×10¹² copies per reaction, would produce the 75.30° C. Tm aligned with the average Tm of susceptible L-DNA (FIG. 25). The optimal L-DNA strand ratio was rounded up from 0.487× (3.665×10¹² melt probe copies per reaction) to 0.5× (3.7625×10¹² melt probe copies per reaction) for ease of sample preparation in the SMASH assay. This method produced near-zero melt differences between drug-susceptible L-DNA and duplexed melt probe bound to wild-type asymmetric PCR product. Specifically, the average melt difference was −0.23±0.21° C. using the QuantStudio™ 5 (mean±SD, FIG. 21A) and −0.08±0.21° C. using the Rotor-Gene® Q (mean±SD, FIG. 22A). It is important to note that although this study sought to match Tms of the L-DNA and the probe-wild-type duplex, it is not critical to do so. Even without tuning the melt probe concentration to make the melt difference zero, for a fixed concentration of melt probe in every sample, the Tm difference will still be a constant in the system and samples can be classified as drug-susceptible when sample Tm difference equals that constant.

Methods: In experiments varying melt probe concentrations, reaction component deviations from standard SMASH assay reactions included 1.881×10¹² (0.25×), 3.763×10¹² (0.5×), 7.525×10¹² (1×), 1.881×10¹³ (2.5×), and 3.763×10¹³ (5×) copies of susceptible melt probe per reaction. The concentrations were selected using 1× as 7.525×10¹² copies per reaction because this copy number matched the theoretical amount of PCR copies formed by the excess primer at 250 nM per reaction. Tuning experiments were performed in the QuantStudio™ 5 and the only sample type tested was wild-type rpoB (n=1 trial in triplicate per melt probe concentration). Logarithmic interpolation of four different melt probe concentrations were used to determine the relationship between copies of melt probe per reaction and melt measurement of duplexed probe to wild-type asymmetric PCR product. The susceptible melt probe copy number with a melt measurement matching that of average susceptible L-DNA was selected. The drug-susceptible L-DNA average Tm was collected across n=1 trial in triplicate across NTC, wild-type, and variant I491F/N/M sample types.

ROC Analysis Using within-Sample Tm Differences on SMASH Sample Sets

### Classification and ROC Analysis Using Multi-Sample Comparison on SMASH Sample Sets

The SMASH assay for rpob codon 491 included two indicators of rifampicin susceptibility in every sample: a susceptible melt probe and a susceptible L-DNA. L-DNA offers numerous advantages to the assay, such as providing an internal melt comparison for susceptibility in every sample, correcting for within-assay variability as a hybridization melt standard, and enabling single-sample classification without relying on multi-sample comparison or historical data. However, the iFRET strategy used to acquire L-DNA fluorescence during melt analysis requires that the real-time PCR instrument be capable of excitation in one optical channel and emission in another. Common real-time PCR instruments, like the LightCycler 4808, QuantStudio™ 39, QuantStudio™ 5 9, and Rotor-Gene® Q10, meet this criterion. In cases where such instrument capabilities are not available or other L-DNA requirements are not met, the SMASH 1491 assay developed in this work can be adapted to be L-DNA-independent.

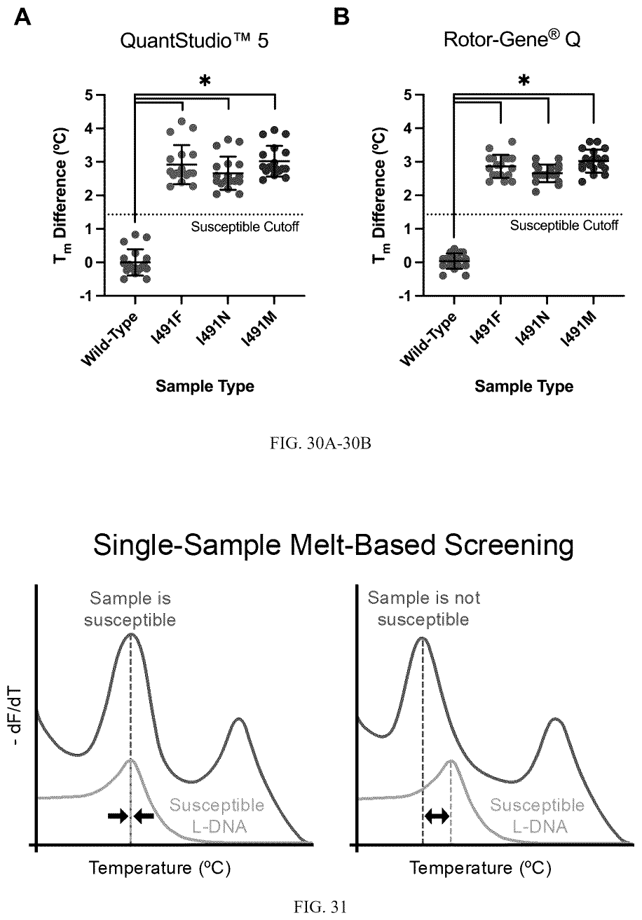

The original QuantStudio™ 5 and Rotor-Gene® Q datasets, which included both melt comparators (melt probe and L-DNA), were re-analyzed using a multi-sample Tm comparison (FIG. 30A-30B)—similar to the André assay's analysis strategy—rather than a within-sample Tm comparison employed in the main report (FIG. 21A and FIG. 22A). Here, Tm difference was calculated as the difference between each sample's probe-product duplex Tm and the sample set's global average Tm of the probe bound to wild-type asymmetric PCR product.

Even without incorporating L-DNA melt data into classification analysis, the SMASH assay still successfully classified all rpoB I491 variants (I491F, I491N, and I491M) as not susceptible and all wild-type rpoB samples as susceptible (FIG. 30A-30B). The assay maintained high performance across two highly calibrated, real-time PCR platforms, the QuantStudio™ 5 (FIG. 30A) and the Rotor-Gene® Q (FIG. 30B). Both PCR systems demonstrated 100% sensitivity and 100% specificity when classifying rifampicin susceptibility (n=6 trials in triplicate per sample type per instrument, FIG. 30A-30B). Successful sample classification is illustrated by 18/18 wild-type rpoB samples below the susceptible cutoff and 54/54 variant I491 samples above the susceptible cutoff, in both the QuantStudio™ 5 sample set (Figure S7A) and the Rotor-Gene® Q sample set (FIG. 30B).

Using ROC analysis (Table 16), QuantStudio™ 5 and Rotor-Gene® Q samples were classified as rifampicin-susceptible using a Tm difference less than 1.43° C. The QuantStudio™ 5 sample set's average Tm differences for wild-type, I491F, I491N, and I491M were 0.00±0.39, 2.92±0.58, 2.66±0.50, and 3.02±0.46, respectively (mean±SD). Using melt difference comparison amongst the QuantStudio™ 5 sample set, there was a significant difference between wild-type and I491F (p<0.0001) but no significant difference between wild-type and I491N or wild-type and I491M (p>0.05, one-way ANOVA, Tukey's post hoc test for multiple comparisons, 9 replicates per sample type, FIG. 30A). The Rotor-Gene® Q sample set's average Tm differences for wild-type, I491F, I491N, and I491M were 0.03±0.23, 2.86±0.35, 2.66±0.26, and 3.02±0.34, respectively (mean±SD). Using melt difference comparison, there was a significant difference between wild-type and I491F (p<0.0001) but no significant difference between wild-type and I491N or wild-type and I491M (p>0.05, one-way ANOVA, Tukey's post hoc test for multiple comparisons, 9 replicates per sample type, FIG. 30B).

Methods: The analyses of multi-sample melt difference and statistical significance were performed independently for the QuantStudio™ 5 and Rotor-Gene® Q using the primary report's original data sets collected for the SMASH assay. A multi-sample melt difference was calculated between the Tm of each sample's probe-product duplex and the sample set's global average Tm of the probe bound to wild-type product. Significance was evaluated using multi-sample melt difference comparison (one-way ANOVA, Tukey's post hoc test for multiple comparisons, significance level of α=0.95) across I491F, I491N, and I491F as compared to wild-type (n=6 trials in triplicate per sample type per instrument). Since true positives were known, the SMASH assay was assessed for its sensitivity and specificity using Receiver Operating Characteristic (ROC) analysis (Wilson/Brown method) across both the QuantStudio™ 5 and Rotor-Gene® Q sample sets (Table 16). Across both instruments' sample sets, a universal Tm difference cutoff point was selected by prioritizing maximized specificity (to decrease the false positive rate, i.e., decrease the misdiagnosis of variant samples as drug-susceptible), followed by maximized sensitivity, when classifying each test sample as drug-susceptible or not. Samples were classified as rifampicin-susceptible using a multi-sample Tm difference less than 1.43° C. for the QuantStudio™ 5 and Rotor-Gene® Q sample sets (n=6 trials in triplicate per sample type per instrument). All statistics were performed in GraphPad Prism version 10.0.

### Results

Across two real-time PCR instruments, the SMASH assay for I491 successfully classified all rpoB codon 491 variants (I491F, I491N, and I491M) as not susceptible and all wild-type rpoB samples as susceptible using single-sample melt-based screening (FIG. 21A and FIG. 22A). Based on Receiver Operating Characteristic (ROC) analysis of the SMASH assay across both the QuantStudio™ 5 and Rotor-Gene® Q sample sets (Table 15), samples were classified as rifampicin-susceptible when the within-sample Tm difference (between L-DNA Tm and the probe-product duplex Tm) was less than 0.83° C. (FIG. 21A and FIG. 22A). Using the QuantStudio™ 5, average within-sample Tm differences for wild-type, I491F, I491N, and I491M samples were −0.23±0.21, 2.68±0.16, 2.55±0.14, and 2.75±0.08, respectively (mean±SD, FIG. 21A). Using the Rotor-Gene® Q, average within-sample Tm differences for wild-type, I491F, I491N, and I491M samples were −0.08±0.21, 2.36±0.48, 1.87±0.31, and 2.39±0.48, respectively (mean±SD, FIG. 22A). Visual alignment and near-zero Tm differences of the susceptible melt duplexes (top left panels of FIG. 21A and FIG. 22A) were achieved by adjusting probe concentration (FIG. 25), though this step was not essential for the within-sample melt comparison strategy. Significant differences were found between wild-type and each of the three variants when the assay was performed in the QuantStudio™ 5 (FIG. 21A) as well as the Rotor-Gene® Q (FIG. 22A, p<0.0001, one-way ANOVA, Tukey's post hoc test for multiple comparisons, n=6 trials in triplicate per sample type per instrument). Both PCR systems demonstrated 100% sensitivity and 100% specificity when classifying rifampicin susceptibility (n=6 trials in triplicate per sample type per instrument, FIG. 21A and FIG. 22A). Successful sample classification is illustrated by 18/18 wild-type rpoB samples below the susceptible cutoff and 54/54 variant I491 samples above the susceptible cutoff, in both the QuantStudio™ 5 (FIG. 21A) and the Rotor-Gene® Q (FIG. 22A) sample sets.

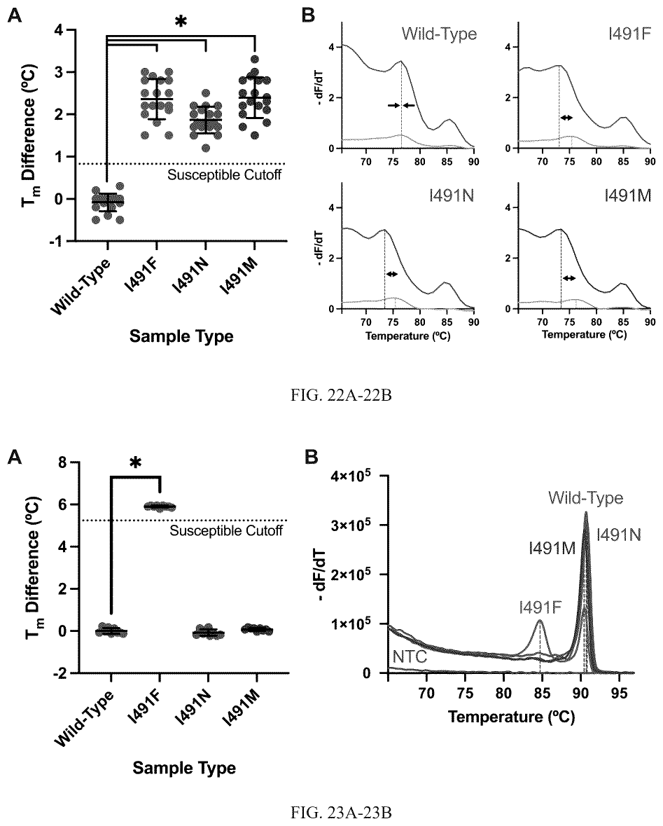

The performance of the SMASH assay for I491 was compared to the reference André I491F assay using the same rpoB test system in the QuantStudio™ 5. While the André I491F assay confirmed successful classification of all wild-type rpoB and I491F variant samples, the expansion of this test to I491N and I491M variant samples resulted in misclassification (FIG. 23A). Per André et al., samples were classified as rifampicin-susceptible using a Tm difference less than 5.24° C. from the global average wild-type melt temperature from the sample set (n=3 trials in triplicate per sample type). As supported by prior literature, melt characteristics differed between wild-type rpoB and variant I491F (FIG. 23B). Variants I491N and I491M, however, had nearly identical melt characteristics to wild-type rpoB despite exhibiting SNV's (FIG. 23B). Average Tm differences for wild-type, I491F, I491N, and I491M were 0.00±0.14, 5.90±0.05, −0.08±0.16, and 0.07±0.07, respectively (mean±SD). Using melt difference comparison, there was a significant difference between wild-type and I491F (p<0.0001) but no significant difference between wild-type and I491N or wild-type and I491M (p>0.05, one-way ANOVA, Tukey's post hoc test for multiple comparisons, 9 replicates per sample type, FIG. 23A). Successful sample classification is illustrated by 9/9 wild-type rpoB samples below the susceptible cutoff and 9/9 variant I491F samples above the susceptible cutoff (FIG. 23A, n=3 trials in triplicate per sample type). Misclassification is illustrated by 9/9 I491N samples and 9/9 I491M samples below the susceptible cutoff (n=3 trials in triplicate per sample type, FIG. 23A).

### DISCUSSION

The incorporation of internal comparators in the SMASH assay enabled each unknown sample to be successfully classified as susceptible or not without the need for comparison to other samples. This single-sample melt-based approach, effectively screened for rifampicin susceptibility amongst wild-type rpoB and three sequence-identified rpoB variants: I491F, I491N, and I491M. The assay achieved 100% sensitivity and 100% specificity in classifying rifampicin susceptibility of synthetic unknown samples (n=6 trials in triplicate per sample type per instrument) across multiple real-time PCR platforms (QuantStudio™ 5 and Rotor-Gene® Q, FIG. 21A and FIG. 22A). Its consistent high performance across two platforms highlights the method's robustness and adaptability, ensuring broader TB screening access using existing instruments—without the need for assay-specific equipment investments.

Notably, a single susceptibility cutoff was applied uniformly across both instruments (Table 15), simplifying the SMASH strategy by eliminating instrument-specific optimizations. This reinforces SMASH's potential for implementation on any real-time PCR system with built-in melt analysis capabilities, offering a broadly applicable, instrument-agnostic solution for rapid rifampicin susceptibility screening.

Prior rpoB 1491 screening efforts relied on either resource-intensive sequencing methods covering the entire rpoB gene or highly-specific real-time melt analysis targeting rpoB nucleotide 1471, the single base location of I491F. In our report, the melt-based I491F-specific André assay was revisited with an expanded variant set, incorporating SNVs at all three positions of codon 491 using rifampicin resistance-related variants I491F, I491N, and I491M. While the Andre I491F assay successfully classified I491F as not susceptible, its selective mechanism of action failed to correctly classify other resistance-related mutations within the rpoB codon 491 hotspot (FIG. 23A). Comprehensive screening of all three nucleotides in codon 491 would require two additional iterations of the Andre assay, each targeting one of the remaining rpoB nucleotides (1472 and 1473). This would necessitate three separate samples to screen for SNVs at all three positions of codon 491—a workflow that adds additional complexity into the TB clinical treatment algorithm.

Key Factors Driving SMASH Assay Success The success of our single-sample melt-based assay is driven by five key features:
(1) including a susceptible melt probe in every sample;
(2) selecting the melt probe sequence to maximize mutation-induced melt shifts;
(3) incorporating a susceptible L-DNA comparator for internal Tm calibration;
(4) adjusting melt probe concentration to align Tms of the two susceptible duplexes (double-stranded L-DNA and the probe-wild-type product duplex); and
(5) collecting melt data from L-DNA and D-DNA on separate optical channels.

These features are detailed in more detail below:

(1) Inclusion of a Susceptible Melt Probe: The susceptible melt probe was incorporated into every sample and designed as the reverse complement of the drug-susceptible rpoB sequence. This ensured the melt probe could hybridize with the asymmetric PCR product excess strand regardless of whether the unknown sample contained a wild-type rpoB sequence.

(2) Optimized Melt Probe Sequence for Maximized Melt Shifts: The susceptible melt probe sequence was strategically selected to maximize the melt shift between its duplex with a wild-type strand and its duplex with an I491F strand (FIG. 24). Consistent with prior literature, increasing the distance of the SNV (I491F at rpoB nucleotide 1471) from the probe's 5′ end increased the melt shift between the probe-I491F and the probe-wild-type duplexes, likely due to greater duplex destabilization caused by the mismatch (FIG. 24). This optimized melt difference facilitated clear discrimination between I491F samples and the susceptible L-DNA melt behavior (FIG. 21A and FIG. 22A). Similarly, mismatched probe-product duplexes from I491N and I491M samples exhibited distinct melt differences from susceptible profiles (FIG. 21A and FIG. 22A).

Since the melt probe sequence defines the SMASH assay's screening region, this 30-base probe span determines which existing or potentially emerging mutations can be identified. The assay may detect other SNVs, beyond the I491F/M/N variants validated here, that cluster within this 30-base region around the codon 491 hotspot, provided a mutation induces a sufficiently distinguishable melt shift. For example, the recently identified I491V and I491L variants, which may contribute to rifampicin resistance, demonstrate a pattern of recurrent mutations in this region. This highlights a use case where SMASH's broad screening capability can be capitalized on. By leveraging the tendency of resistance-related mutations to cluster, the SMASH strategy has the potential to detect both established, sequence-identified mutations—which are expected to become more prevalent—and emerging mutations that may arise over time.

(3) Inclusion of a Susceptible L-DNA Comparator for Internal Calibration: The double-stranded L-DNA comparator was designed to match the duplex sequence of the susceptible probe hybridized to a wild-type asymmetric PCR product, making L-DNA an effective melt comparator for susceptibility classification. SMASH incorporates L-DNA in every sample to achieve a reagent-based calibration, enabling single-sample classification without requiring multi-sample comparisons, while also correcting for potential assay variations across reaction wells or plates. Similar L-DNA-based strategies have been successfully employed in prior work, including a 56-base double-stranded L-DNA melt comparator for isoniazid susceptibility.

Consistent with previous research, the melt characteristics of susceptible-sequence D-DNA (melt probe bound to wild-type asymmetric PCR product) and L-DNA (double-stranded melt comparator) were nearly identical (top left panels in FIG. 21B and FIG. 22B). SMASH melt analysis compared the sequence-specific Tm of the L-DNA comparator to that of the probe-product duplex. A Tm shift in the probe-product duplex relative to the susceptible L-DNA comparator indicated the presence of a SNV in the unknown sample (rpoB I491 variants in FIG. 21A-21B and FIG. 22A-22B). This internal melt comparison strategy facilitated high-performance single-sample classification.

(4) Adjusting Melt Probe Concentration for Visual Melt Alignment: Single-sample classification was further enhanced by adjusting the melt probe concentration to align the melt temperatures of the two susceptible duplexes: double-stranded L-DNA and the susceptible melt probe bound to the wild-type asymmetric PCR product.

While achieving a near zero Tm difference simplified visual interpretation of melt curves as susceptible or not (FIG. 21B and FIG. 22B), matching was not strictly necessary. The Tm difference remains constant for a fixed probe concentration, allowing reliable sample classification based on this invariant offset.

To optimize melt matching, the susceptible melt probe concentration was adjusted so that the Tm of its duplex with the wild-type asymmetric PCR product aligned with that of the double-stranded L-DNA comparator (FIG. 25). Previous studies have demonstrated that total DNA concentration and strand ratio affect the melt temperature, a principle applied in our previous L-DNA-based work where the susceptible L-DNA concentration was adjusted so L-DNA melt characteristics aligned with that of the wild-type PCR amplicon. In the present study, the susceptible melt probe concentration was reduced to ensure that the Tm of its duplex to wild-type asymmetric PCR product matched L-DNA Tm (FIG. 25). This empirical melt match between susceptible duplexes (top left panels in FIG. 21B and FIG. 22B) only needed to be completed one time in test development, and contributed to successful implementation of L-DNA for rifampicin susceptibility screening.

(5) Collecting L-DNA and D-DNA Melt Data on Separate Optical Channels: To facilitate single-sample classification and discriminate between L-DNA and D-DNA melting behavior within one reaction, the SMASH assay collects L-DNA fluorescent melt data on a separate optical channel from D-DNA. This distinction was necessary because all readily available intercalating dyes do not discriminate between enantiomeric DNA. In our related L-DNA-based isoniazid susceptibility assay, intercalating crosstalk was overcome by labeling double-stranded L-DNA with a Texas Red fluorophore and a quencher. This enabled L-DNA melt monitoring in a separate optical channel from the green intercalating dye used for the D-DNA PCR products. However, the fluorescence resonance energy transfer (FRET) mechanism between the fluorophore and quencher during melting causes a rise in donor fluorescence, resulting in a negative melt curve. Since many real-time PCR instruments are unable to determine Tm from such curves, this design required post-processing steps to accurately to determine L-DNA Tm, adding extra steps to the workflow.

To overcome this challenge, the present study implemented intercalated FRET (iFRET) to capture L-DNA melt data. Probe-product duplex melting was monitored on the green optical channel via SYBR® Green I intercalating dye, while L-DNA melting was monitored on the orange optical channel using iFRET, with SYBR® Green I as the FRET donor and Texas Red (labeled on the L-DNA forward strand) as the FRET acceptor. Unlike traditional fluorophore-quencher FRET, iFRET results in a drop in acceptor fluorescence during melting, generating a positive melt curve that can be automatically analyzed by built-in PCR instrument software to calculate Tm. This eliminates the need for post-processing and simplifies the assay workflow.

Beyond workflow efficiency, iFRET offers cost and accuracy advantages. iFRET is more cost-effective than traditional fluorophore-quencher FRET designs since it requires only a single fluorophore label on L-DNA. Furthermore, iFRET preserves melt measurement accuracy; Tm values from iFRET were equivalent to those obtained via intercalating dye measurements, as confirmed by L-DNA melting in no template control (NTC) samples (FIG. 29).

For iFRET to function, the L-DNA fluorophore must have sufficient spectral overlap with the intercalating dye. Additionally, the real-time PCR instrument must support excitation in one optical channel and emission in another. Common real-time PCR instruments such as the LightCycler 48034, QuantStudio™ 335, QuantStudio™ 5 35, and Rotor-Gene® Q36 meet this requirement.

Extending the Strategy to Other Hotspots The SMASH screening strategy presented in this report is highly generalizable and can be adapted to other mutation hotspots. Its implementation depends on sequencing efforts to identify mutations in emerging hotspots and on clinical and epidemiological evidence linking these sequence-identified variants to pathogenic impact. Once such associations are established, hotspot-specific SMASH can be implemented in four steps. First, PCR primers are designed to amplify the target screening region. Second, a susceptible melt probe is selected to define the exact nucleotide screening span and optimized to maximize mutation-induced melt shifts. Third, a left-handed L-DNA sequence is synthesized to mimic the susceptible duplex formed by the melt probe hybridized to the wild-type asymmetric PCR product. The screening region length is constrained by two factors: the synthesis limitations of L-DNA (biomers.net) and the decreasing detectability of SNV-induced melt temperature shifts as sequence length increases. Fourth, the melt probe concentration is adjusted, if desired, to align the melt temperatures of the probe-wild-type product duplex with the susceptible L-DNA. While this step simplifies visual interpretation of melt curves as susceptible or not, it is not strictly necessary. The Tm difference remains constant for a fixed probe concentration, ensuring reliable sample classification even without matching the melt curves.

Resource-constrained Alternative Designs Less than 1% of the SMASH assay's reagent cost is attributed to L-DNA. The L-DNA primarily serves as a susceptible melt comparator in the SMASH assay for I491. Our previous work also suggests that L-DNA functions as an internal melt calibrator, correcting for within-assay variability (such as well-to-well or plate-to-plate differences) that could otherwise reduce classification performance. This within-sample correction compensates for systematic hybridization changes caused by sample preparation errors or other sources of variability, such as culture media carryover, extraction errors, kit-to-kit master mix differences, or sample-to-sample salt concentration variability resulting from reagent pipetting errors. The inclusion of L-DNA for reagent-based calibration suggests that, even though the SMASH assay was tested on two state-of-the-art calibrated instruments, highly calibrated equipment may not be essential for successful SMASH assay implementation. Instead, the calibration function is provided by L-DNA itself. While L-DNA-based calibration effectively corrects for within-assay variability, it is important to note that within-sample Tm differences may still vary slightly between instruments, as demonstrated in this study across two different platforms.

Despite the benefits of incorporating an L-DNA comparator in every sample, certain use-cases for I491 screening may arise where iFRET-capable instruments are unavailable or other L-DNA requirements are not met. The SMASH I491 assay developed in this study can be adapted to be L-DNA-independent while still maintaining high performance (FIG. 30A-30B). This alternative approach demonstrates how the SMASH I491 screening assay can be analyzed using a traditional melt probe method with multi-sample comparison. When L-DNA melt data is excluded from classification analysis on highly-calibrated, real-time PCR instruments (QuantStudio™ 5 and Rotor-Gene® Q), multi-sample Tm comparison-similar to the strategy used to analyze the André assay sample set-still consistently achieved 100% sensitivity and 100% specificity in classifying rifampicin susceptibility in unknown synthetic samples (n=6 trials in triplicate per sample type per instrument, FIG. 30A-30B). In this case, Tm difference was calculated as the difference between each sample's probe-product duplex Tm and the sample set's global average Tm of the probe bound to wild-type product. However, this L-DNA-independent approach would rely on historical melt data for classification, eliminating single-sample classification capabilities, and may be more prone to within-assay variability, potentially compromising accuracy-whereas L-DNA could correct for such variability as an internal melt standard.

This study establishes SMASH as a promising single-sample melt-based strategy for mutation screening at rpoB codon 491, with strong proof of concept using a synthetic rpoB system.

The strong selective pressure exerted by anti-TB drugs drives the emergence and spread of drug-resistant MTB strains. As resistance-related mutations accumulate, their prevalence and clinical significance are expected to increase, making early and accurate detection critical.

MTB resistance mutations often cluster in well-characterized regions, such as the RRDR of rpoB, which accounts for approximately 96% of rifampicin resistance, and the quinolone resistance-determining region (QRDR) of gyrA and gyrB, responsible for 50-90% and 7% of fluoroquinolone resistance, respectively. These clustering patterns underscore the need for targeted hotspot screening.

SMASH's conservative design, which scans regions surrounding known hotspots, enhances its ability to detect both established resistance mutations and newly emerging variants clustering within these critical regions. By leveraging the natural aggregation of resistance mutations, SMASH serves as both a powerful screening tool and an anticipatory strategy for resistance surveillance. Expanding SMASH to additional MTB regions with well-documented hotspot aggregation could further improve resistance monitoring.

Currently, SMASH is applicable when target mutations serve as unique classifiers of disease susceptibility. A key future advancement would be multiplexing SMASH to simultaneously screen multiple hotspots critical to clinical outcomes. For example, a multiplexed SMASH assay could assess rifampicin susceptibility by screening both the RRDR and rpoB codon 491 in a single reaction, improving diagnostic efficiency.

Beyond TB, SMASH's adaptability can facilitate the detection of clinically significant mutations in other genetic hotspots linked to disease susceptibility. For instance, TP53 mutations are associated with malignant progression and chemoresistance, while gyrA mutations contribute to drug resistance in bacterial pathogens such as corynebacteria and salmonella. By facilitating targeted screening of these regions, SMASH has the potential to deepen our understanding of disease etiology, refine therapeutic and diagnostic strategies, and support the advancement of personalized medicine.

### CONCLUSION

The present disclosure establishes SMASH as a new single-sample strategy for screening sequence-identified mutations with clinical significance. SMASH was successfully applied to rifampicin susceptibility screening at the commercially unaddressed rpoB 491 mutation hotspot, while unexpectedly achieving 100% sensitivity and 100% specificity. Beyond TB drug resistance, SMASH offers a generalizable framework adaptable to other disease susceptibility applications, with the capacity to improve diagnostic accuracy and inform clinical decision-making.

All of the compositions and methods disclosed and claimed herein can be made and executed without undue experimentation in light of the present disclosure. While the compositions and methods of this disclosure have been described in terms of preferred embodiments, it will be apparent to those of skill in the rut that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A method of detecting sequence variation based on a comparison of melt temperature between an unknown double stranded D-DNA molecule and a reference double stranded D-DNA molecule with a first reference melt temperature and with a first reference elapsed melt time, the method comprising:
(a) providing a double stranded L-DNA molecule with a second reference melt temperature and with a second reference elapsed melt time;
(b) obtaining a first observed melt temperature and a first observed elapsed melt time for the double stranded L-DNA molecule under assay conditions;
(c) obtaining a second observed melt temperature and a second observed elapsed melt time for the unknown double stranded D-DNA molecule under assay conditions identical to step (b); and
(d) determining the difference between the first observed melt temperature provided by step (b) and the second observed melt temperature provided by step (c);
wherein, when the difference of step (d) is not equal to the difference between the first reference melt temperature and the second reference melt temperature, then the unknown double stranded D-DNA molecule is identified as having a sequence variation relative to the reference double stranded D-DNA molecule.

2. The method of claim 1, comprising detecting sequence variation based on a comparison of the elapsed melt time between the unknown double stranded D-DNA molecule and the reference double stranded D-DNA molecule with the first reference elapsed melt time, under assay conditions identical to step (b); and further comprising:
(e) determining the difference between the elapsed melt times provided by step (b) and step (c);
wherein, when the difference in step (e) is not equal to the difference between the first reference elapsed melt time and the second reference elapsed melt time, then the unknown double stranded D-DNA molecule is identified as having a sequence variation relative to the reference double stranded D-DNA molecule.

3. The method of claim 1, wherein the melt temperature and/or elapsed melt time are obtained using a calibrated instrument or a non-calibrated instrument.

4. The method of claim 1, wherein the unknown double stranded D-DNA molecule is provided using real time PCR performed in the presence of the double stranded L-DNA reference molecule in the same reaction.

5. The method of claim 1, wherein the sequence variation between the unknown double stranded D-DNA molecule and the reference double stranded D-DNA molecule is a single base change.

6. The method of claim 1, wherein the double stranded L-DNA molecule and the reference D-DNA molecule do not have the same sequence.

7. The method of claim 1, wherein the melt temperature or elapsed melt time of the unknown double stranded D-DNA molecule is determined using an intercalating dye.

8. The method of claim 7, wherein the intercalating dye is selected from the group consisting of SYBR® Gold, SYBR® Green, EvaGreen®, SYTO™ 82, SYTO™ 64, SYTO™ 9, and LCGreen® dyes.

9. The method of claim 1, wherein the double stranded L-DNA is end-labeled on either the forward or reverse strand with a dye, wherein the double stranded L-DNA is end-labeled with quencher on the strand opposite to the dye-end-labeled strand, and wherein the unknown double stranded D-DNA is labeled with an intercalating dye that is not impacted by the quencher.

10. The method of claim 9, wherein the quencher-labeled strand of the L-DNA is present in a molar excess relative to the fluorophore labeled L-DNA strand.

11. The method of claim 1, wherein the double stranded L-DNA is end-labeled on either the forward or reverse strand with a dye, and wherein excitation is provided by a compatible intercalating dye.

12. The method of claim 1, wherein the concentration or the ratio of the forward: reverse strands of the double stranded L-DNA is adjusted to provide a desired melt temperature or elapsed melt time.

13. The method of claim 12, wherein the double stranded L-DNA concentration and/or strand ratio are adjusted according to the Van't Hoff equation.

14. The method of claim 1, wherein the melt temperatures or elapsed melt times are obtained simultaneously in the same reaction.

15. The method of claim 1, wherein the unknown D-DNA is a template dependent amplification product.

16. The method of claim 1, wherein multiple distinct double stranded L-DNA sequences with multiple distinct melt temperatures or multiple distinct elapsed melt times are used together in the same reaction.

17. The method of any claim 1, wherein the reference double stranded D-DNA has the same sequence as a natural D-DNA unique to a drug-resistant pathogen.

18. The method of claim 17, wherein the drug-resistant pathogen is Mycobacterium tuberculosis, and the reference double stranded D-DNA sequence comprises
(SEQ ID NO: 1)
GGCACCAGCCAGCTGAGCCAATTCATGGACCAGAACAACCCGCT
GTCGGGGTTGACCCACAAGCGCCGACTGTCGG CGCTG.

19. A method of detecting sequence variation between an unknown double stranded D-DNA molecule and a reference double stranded D-DNA molecule, the method comprising:
(a) providing a melt probe having reverse complementarity to a drug-susceptible sequence in the unknown double stranded D-DNA molecule relative to a corresponding sequence in the reference double stranded D-DNA molecule;
(b) providing a double stranded L-DNA molecule;
(c) optionally adjusting the concentration of the melt probe and/or adjusting the concentration or the ratio of the forward: reverse strands of the double stranded L-DNA so that the melt temperatures or reference elapsed melt times are about identical between a melt probe: reference asymmetric PCR product duplex and the double stranded L-DNA molecule;
(d) performing asymmetric PCR to provide one or more asymmetric PCR products of the unknown double stranded D-DNA molecule, wherein one or more melt probe: asymmetric PCR product duplexes are formed;
(e) obtaining a first observed melt temperature and a first observed elapsed melt time for the double stranded L-DNA molecule under assay conditions;
(f) obtaining a second observed melt temperature and a second elapsed melt time for the one or more melt probe: asymmetric PCR product duplexes under assay conditions identical to step (e); and
(g) determining the difference between the first observed melt temperature or first elapsed melt time provided by step (e) and the second observed melt temperature or second elapsed melt time provided by step (f);
wherein, if there is no difference in step (g), then the unknown double stranded D-DNA molecule is identical to the reference double stranded D-DNA molecule; and
wherein, if there is a difference in step (g), then the unknown double stranded D-DNA molecule is identified as having a sequence variation relative to the reference double stranded D-DNA molecule.

20. The method of claim 19, wherein the forward strand of the double stranded L-DNA molecule is identical in sequence and in length to the melt probe.

21. A method of detecting sequence variation in an unknown double stranded D-DNA molecule as compared to a reference double stranded D-DNA molecule which does not involve the use of L-DNA, the method comprising:
(a) providing a control probe having reverse complementarity to a first sequence that is identical between the unknown double stranded D-DNA molecule and the reference double stranded D-DNA molecule;
(b) providing a melt probe having reverse complementarity to a drug-susceptible sequence in the unknown double stranded D-DNA molecule relative to a corresponding sequence in the reference double stranded D-DNA molecule;
(c) adjusting the concentration and/or ratios of the first probe and second probe so that the melt temperatures or elapsed melt times are identical between the control probe and the susceptible melt probe;
(d) performing asymmetric PCR to provide one or more asymmetric PCR products;
(e) obtaining a first melt temperature and a first elapsed melt time for a control probe: asymmetric PCR product duplex under assay conditions;
(f) obtaining a second melt temperature and a second elapsed melt time for a susceptible melt probe: asymmetric PCR product duplex under assay conditions identical to step (e); and
(g) determining the difference between the melt temperatures or elapsed melt times provided by step (e) and step (f);
wherein, if there is no difference in step (g), then the unknown double stranded D-DNA molecule is identical to the reference double stranded D-DNA molecule; and
wherein, if there is a difference step (g), then the unknown double stranded D-DNA molecule is identified as having a sequence variation relative to the reference double stranded D-DNA molecule.
